(19) United States
(12) Patent Application Publication
(10) Pub. No.: US 2026/0202291 A1
(43) Pub. Date: Jul. 16, 2026

(54) Detection of Target Molecules in Dried Biologic Matrices

(71) Applicant: Somalogic Operating Co., Inc., Boulder, CO (US)

(72) Inventors: Weston J. Smith, Boulder, CO (US); Daniel W. Drolet, Boulder, CO (US)

(73) Assignee: SomaLogic Operating Co., Inc., Boulder, CO (US)

(21) Appl. No.: 19/424,331

(22) Filed: Dec. 18, 2025

Related U.S. Application Data
(63) Continuation of application No. PCT/US24/36674, filed on Jul. 3, 2024.
(60) Provisional application No. 63/525,341, filed on Jul. 6, 2023.
(60) Provisional application No. 63/530,113, filed on Aug. 1, 2023.

(51) Int. Cl. G01N 1/40; G01N 33/53; G01N 33/543

(52) U.S. Cl. CPC G01N 1/4022; G01N 33/5308; G01N 33/54306; G01N 2001/4027

(57) ABSTRACT

Methods for detection of target molecules in dried biologic matrices in proteomic based assays are provided. Such methods have a wide utility in proteomic applications for research and development, diagnostics and therapeutics by providing methods for the preparation of dried biologic matrices for detection of target molecules in a proteomic assay and to methods of detecting one or more target molecules from dried biologic matrices in a proteomic assay.

### CROSS-REFERENCE TO RELATED APPLICATIONS

The present application is a continuation of International Application No. PCT/US2024/036674, filed Jul. 3, 2024, which claims the benefit of priority of US Provisional Patent Application Nos. 63/525,341, filed Jul. 6, 2023, and 63/530,113, filed Aug. 1, 2023, each of which is incorporated by reference herein in its entirety for any purpose.

### FIELD

The present disclosure relates generally to the field of proteomic assays, and methods for detection of target molecules in dried biologic matrices. Such methods have a wide utility in proteomic applications for research and development, diagnostics, and therapeutics. Specifically, methods are provided for the preparation of dried biologic matrices for detection of target molecules in a proteomic assay and to methods of detecting target molecules from dried biologic matrices in a proteomic assay.

### BACKGROUND

Assays directed to the detection and quantification of physiologically significant molecules in biological samples and other sample types are important tools in scientific research and in the health care field. For example, multiplex array assays employ surface bound probes to detect target molecules in a sample. The surface-bound probes may be oligonucleotides, peptides, polypeptides, proteins, antibodies, affibodies, aptamers or other molecules (collectively biopolymers) capable of binding with target molecules from the sample. These binding interactions are the basis for many of the methods and devices used in a variety of different fields, e.g., genomics, transcriptomics and proteomics.

Assays using solution-based samples typically require maintaining the solution-based samples at −80° C. prior to the assay, including shipping and storage. In addition, frozen solution-based samples that include cells, such as whole blood, may have lysis of the cells with the freeze-thaw sample processing, the samples may have insufficient homogenization of the cells. Improper storage of solution-based samples and incomplete homogenization of cells within solution has the potential to introduce pre-analytical variation on the target molecules withing the solution-based samples.

This disclosure describes methods to eliminate the need for −80° C. shipping and storage of samples prior to the assay. This disclosure describes methods to eliminate or reduce incomplete homogenization of cells within solution. This disclosure describes methods for detecting target molecules within the samples extracted from dried biologic matrices.

### SUMMARY

The foregoing and other objects, features, and advantages of the disclosure will become more apparent from the following detailed description, which proceeds with reference to the accompanying figures.

Certain non-limiting exemplary embodiments are as follows:

In some embodiments, methods of preparing a biological sample for a multiplex assay are disclosed. The methods comprise depositing the biological sample comprising a plurality of target molecules onto a collection device, drying the biological sample on the collection device for a period of time to stabilize the dried sample prior to any temperature fluctuation, wherein the biological sample is dried for at least 4 hours at about room temperature or below room temperature so that target molecules in the dried biological sample are detectable in the multiplex assay.

In some embodiments, the biological sample is dried at about room temperature, 4° C.-8° C., or −20° C. In some embodiments, the dried biological sample is stored at about room temperature or below room temperature prior to detection in the multiplex assay. In some embodiments, the dried biological sample is stored at about 4° C.-8° C., or −20° C.

In some embodiments, the biological sample is dried for at least 6 hours, at least 8 hours, at least 10 hours, at least 12 hours, at least 14 hours, at least 16 hours, at least 18 hours, at least 20 hours, at least 22 hours, one day, at least two days, or at least three days.

In some embodiments, the biological sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid. In some embodiments, the biological sample is selected from plasma, serum, urine, and whole blood. In some embodiments, the plurality of target molecules are selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

In some embodiments, the dried biological sample is homogenized by the drying process. In some embodiments, the plurality of target molecules are extractable from the collection device after the biological sample is dried and detectable in the multiplex assay.

In some embodiments, methods of detecting a plurality of target molecules are disclosed. The methods comprise extracting target molecules in a dried biological sample from a collection device, diluting the extracted target molecules into a first dilution and a second dilution, contacting the first dilution with a first capture reagent to form a first capture reagent affinity complex with its target molecule if the target molecule is present in the first dilution, contacting the second dilution with a second capture reagent to form a second capture reagent affinity complex with its target molecule if the target molecule is present in the second dilution, incubating the first and second dilution samples separately to allow capture reagent affinity complex formation; wherein each of the first capture reagent affinity complex and the second capture reagent affinity complex are immobilized on separate first solid supports, releasing and capturing the first capture reagent affinity complex on a second solid support, after releasing the first capture reagent affinity complex, releasing and capturing the second capture reagent affinity complex on the second solid support; and detecting for the presence of or determining the level of the first capture reagent, second capture reagent of the first or second capture reagent affinity complexes, or the presence or amount of the first or second capture reagent affinity complexes.

In some embodiments, the target molecules are extracted from the collection device in a formulation for at least 5 minutes. In some embodiments, the target molecules are extracted from the collection device in a formulation for at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 70 minutes, at least 80 minutes, at least 90 minutes, at least 100 minutes, at least 110 minutes, at least 120 minutes, at least 130 minutes, at least 140 minutes, at least 150 minutes, at least 160 minutes, at least 170 minutes, at least 180 minutes, at least 190 minutes, or at least 200 minutes.

In some embodiments, the formulation comprises a buffering agent, one or more salts, a chelating agent, a protease inhibitor, a non-ionic surfactant, and an oligonucleotide.

In some embodiments, the one or more salts are each independently selected from a sodium salt, a potassium salt and a magnesium salt.

In some embodiments, the one or more salts comprise a sodium salt, a potassium salt and a magnesium salt. In some embodiments, the sodium salt is NaCl, the potassium salt is KCl and the magnesium salt is MgCl2. In some embodiments, NaCl in the formulation is at a concentration of from about 10 mM to about 500 mM, or from about 50 mM to about 250 mM, or from about 100 mM to about 200 mM, or from about 75-125 mM, or about 100 mM. In some embodiments, the KCl in the formulation is at a concentration of from about 0.5 mM to about 30 mM, or from about 1 mM to about 20 mM, or from about 2 mM to about 15 mM, or from about 4 mM to about 10 mM, or about 5 mM. In some embodiments, the MgCl2 in the formulation is at a concentration of from about 0.5 mM to about 30 mM, or from about 1 mM to about 20 mM, or from about 2 mM to about 15 mM, or from about 4 mM to about 10 mM, or about 8 mM.

In some embodiments, the buffering agent is selected from HEPES, IVIES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS. In some embodiments, the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 60 mM, or about 50 mM.

In some embodiments, the chelating agent is selected from EGTA, EDTA, DTPA, BAPTA, DMPS and ALA. In some embodiments, the chelating agent in the formulation is at a concentration of about 0.1 mM to about 10 mM, or from about 0.5 mM to about 5 mM, or about 1.25 mM.

In some embodiments, the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80). In some embodiments, the nonionic surfactant is from about 0.1% to about 5% of the formulation, or about 0.2% to about 4% of the formulation, or from about 0.3% to about 3% of the formulation, or from about 0.4% to about 2% of the formulation, or about 0.5%, or about 1.5% of the formulation, or about 1.2% of the formulation, volume for volume.

In some embodiments, the pH of the formulation is from about pH 5 to about pH 9, or from about pH 6 to about pH 8, or from about pH 7 to about pH 7.9, or about pH 7.5.

In some embodiments, the formulation comprises 50 mM HEPES, 100 mM NaCl, 5 mM KCl, 8 mM MgCl2, 1.25 mM EGTA and 1.2% Tween-20.

In some embodiments, the formulation has a pH of about 7.5.

In some embodiments, the protease inhibitor is a reversible protease inhibitor. In some embodiments, the protease inhibitor inhibits proteases selected from trypsin, plasmin and thrombin. In some embodiments, the protease inhibitor is an inhibitor of serine protease. In some embodiments, the protease inhibitor is benzamidine. In some embodiments, the protease inhibitor in the formulation is at a concentration of about 0.1 mM to about 10 mM, or from about 0.5 mM to about 5 mM, or about 1.2 mM.

In some embodiments, the oligonucleotide is a single stranded oligonucleotide. In some embodiments, the oligonucleotide is from 20 to 100 nucleotides in length, or from 25 to 80 nucleotides in length, or from 25 to 70 nucleotides in length or from 25 to 50 nucleotides in length or about 30 nucleotides in length. In some embodiments, the oligonucleotide comprises one or more modified nucleotides. In some embodiments, the oligonucleotide comprises one or more C-5 modified pyrimidines. In some embodiments, the oligonucleotide comprises the sequence [(A-C-X-X)7-A-C], wherein X is a BndU. In some embodiments, the oligonucleotide in the formulation has a concentration of from 5 μM to 100 μM or from 10 μM to 80 μM or from 20 μM to 60 μM or from 30 μM to 50 μM or about 75 μM or about 37 μM.

In some embodiments, the biological sample has been dried for at least 4 hours at about room temperature or below room temperature. In some embodiments, the biological sample has been dried at about room temperature, 4° C.-8° C., or −20° C. In some embodiments, the dried biological sample has been stored at about room temperature or below room temperature prior to detection. In some embodiments, the dried biological sample has been stored at about 4° C.-8° C., or −20° C. prior to detection.

In some embodiments, the biological sample has been dried for at least 6 hours, at least 8 hours, at least 10 hours, at least 12 hours, at least 14 hours, at least 16 hours, at least 18 hours, at least 20 hours, at least 22 hours, at least one day, at least two days, or at least three days.

In some embodiments, the biological sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid. In some embodiments, the biological sample is selected from plasma, serum, urine, and whole blood. In some embodiments, the plurality of target molecules are selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

In some embodiments, the dried biological sample has been homogenized by the drying process.

In some embodiments, the first and second capture reagent-target molecule affinity complexes are non-covalent complexes.

In some embodiments, the first dilution is a dilution of the eluted target molecule sample of from 0.001% to 0.1% and the second dilution is a dilution of the eluted target molecule sample of from 0.1% to 10%.

In some embodiments, the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%. In some embodiments, the first dilution is a dilution of the test sample of from 0.001% to 0.009% (or 0.001%, 0.002%, 0.003%, 0.004%, 0.005%, 0.006%, 0.007%, 0.008% or 0.009%) or is from 0.002% to 0.008%, or is from 0.003% to 0.007% or is about 0.005%; and the second dilution is a dilution of the test sample of from 5% to 39% (or is 5%, 6%, 7%, 8%, 9%, 10%, 11%, 12%, 13%, 14%, 1, 1⁶%, 1⁷%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%. In some embodiments, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the second dilution is a dilution of the test sample of from 5% to 39% (or is 5%, 6%, 7%, 8%, 9%, 10%, 11%, 12%, 13%, 14%, 1, 1⁶5, 7%, 18%, %19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%. In some embodiments, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%. In some embodiments, the first dilution is a dilution of the test sample of from 5% to 39% (or is 5%, 6%, 7%, 8%, 9%, 10%, 11%, 12%, 13%, 14%, 1, 1⁶%, 1⁷%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%. In some embodiments, the first dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%.

In some embodiments, the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%. In some embodiments, the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%. In some embodiments, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%, and the second dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%. In some embodiments, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%. In some embodiments, the first dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%. In some embodiments, the first dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%.

In some embodiments, the method further comprises contacting a third dilution sample with a third capture reagent, wherein a third capture reagent affinity complex is formed by the interaction of the third capture reagent with its target molecule if the target molecule is present in the third dilution sample; wherein the third dilution sample is incubated separately from the first and second dilution samples to allow capture reagent affinity complex formation of the third aptamer with its target molecule. In some embodiments, the method further comprises after releasing the second capture reagent affinity complex, releasing and capturing the third capture reagent affinity complex on the second solid support. In some embodiments, the method further comprises detecting for the presence of or determining the level of the third capture reagent of the third capture reagent affinity complex, or the presence or amount of the third capture reagent affinity complex.

In some embodiments, the third dilution is a different dilution from the first dilution and/or the second dilution of the same eluted target molecule sample. In some embodiments, the third dilution is a dilution of the eluted target molecule sample of from 0.001% to 0.1%. In some embodiments, the third dilution is a dilution of the eluted target molecule sample of from 0.001% to 40%.

In some embodiments, the third dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), from 15% to 30%, from 15% to 25%, about 20%; from 0.01% to 1% (or 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%), from 0.1% to 0.8%, from 0.2% to 0.75%, about 0.5%; and from 0.001% to 0.009% (or 0.001%, 0.002%, 0.003%, 0.004%, 0.005%, 0.006%, 0.007%, 0.008% or 0.009%), or from 0.002% to 0.008%, from 0.003% to 0.007%, about 0.005%. In some embodiments, the third dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%; or the third dilution is from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%; or the third dilution is from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%.

In some embodiments, the first capture reagent and the second capture reagent are, independently, selected from an aptamer or an antibody. In some embodiments, the third capture reagent is selected from an aptamer or an antibody. In some embodiments, each of the first capture reagent, the second capture reagent, and the third capture reagent is an aptamer. In some embodiments, each aptamer independently comprises at least one 5-position modified pyrimidine. In some embodiments, the at least one 5-positon modified pyrimidine comprises a linker at the 5-position of the pyrimidine and a moiety attached to the linker.

In some embodiments, the linker is selected from amide linker, a carbonyl linker, a propynyl linker, an alkyne linker, an ester linker, a urea linker, a carbamate linker, a guanidine linker, an amidine linker, a sulfoxide linker, and a sulfone linker.

In some embodiments, the moiety is a hydrophobic moiety. In some embodiments, the moiety is selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, a benzofuranyl moiety, phenylbenzyl moiety, a 4-phenoxybenzyl moiety, a diphenylpropyl moiety, and a benzhydryl moiety. In some embodiments, the pyrimidine of the 5-position modified pyrimidine is a uridine, cytidine or thymidine.

In some embodiments, the detecting for the presence or the determining of the level of the dissociated first and second capture reagents is performed by PCR, mass spectrometry, nucleic acid sequencing, next-generation sequencing (NGS) or hybridization.

In some embodiments, methods for preparing a liquid sample are provided. The methods comprise drying the sample at a constant temperature of from −20° C. to room temperature for at least four (4) hours to create a dried sample, and reconstituting the dried sample with a formulation comprising a buffering agent, one or more salts, a chelating agent, a protease inhibitor, a non-ionic surfactant, and an oligonucleotide.

In some embodiments, the one or more salts are each independently selected from a sodium salt, a potassium salt and a magnesium salt.

In some embodiments, the one or more salts comprise a sodium salt, a potassium salt and a magnesium salt. In some embodiments, the sodium salt is NaCl, the potassium salt is KCl and the magnesium salt is MgCl2. In some embodiments, NaCl in the formulation is at a concentration of from about 10 mM to about 500 mM, or from about 50 mM to about 250 mM, or from about 100 mM to about 200 mM, or from about 75-125 mM, or about 100 mM. In some embodiments, the KCl in the formulation is at a concentration of from about 0.5 mM to about 30 mM, or from about 1 mM to about 20 mM, or from about 2 mM to about 15 mM, or from about 4 mM to about 10 mM, or about 5 mM. In some embodiments, the MgCl2 in the formulation is at a concentration of from about 0.5 mM to about 30 mM, or from about 1 mM to about 20 mM, or from about 2 mM to about 15 mM, or from about 4 mM to about 10 mM, or about 8 mM.

In some embodiments, the buffering agent is selected from HEPES, IVIES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS. In some embodiments, the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 60 mM, or about 50 mM.

In some embodiments, the chelating agent is selected from EGTA, EDTA, DTPA, BAPTA, DMPS and ALA. In some embodiments, the chelating agent in the formulation is at a concentration of about 0.1 mM to about 10 mM, or from about 0.5 mM to about 5 mM, or about 1.25 mM.

In some embodiments, the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80). In some embodiments, the nonionic surfactant is from about 0.1% to about 5% of the formulation, or about 0.2% to about 4% of the formulation, or from about 0.3% to about 3% of the formulation, or from about 0.4% to about 2% of the formulation, or about 0.5%, or about 1.5% of the formulation, or about 1.2% of the formulation, volume for volume.

In some embodiments, the pH of the formulation is from about pH 5 to about pH 9, or from about pH 6 to about pH 8, or from about pH 7 to about pH 7.9, or about pH 7.5.

In some embodiments, the formulation comprises 50 mM HEPES, 100 mM NaCl, 5 mM KCl, 8 mM MgCl2, 1.25 mM EGTA and 1.2% Tween-20.

In some embodiments, the formulation has a pH of about 7.5.

In some embodiments, the protease inhibitor is a reversible protease inhibitor. In some embodiments, the protease inhibitor inhibits proteases selected from trypsin, plasmin and thrombin. In some embodiments, the protease inhibitor is an inhibitor of serine protease. In some embodiments, the protease inhibitor is benzamidine. In some embodiments, the protease inhibitor in the formulation is at a concentration of about 0.1 mM to about 10 mM, or from about 0.5 mM to about 5 mM, or about 1.2 mM.

In some embodiments, the oligonucleotide is a single stranded oligonucleotide. In some embodiments, the oligonucleotide is from 20 to 100 nucleotides in length, or from 25 to 80 nucleotides in length, or from 25 to 70 nucleotides in length or from 25 to 50 nucleotides in length or about 30 nucleotides in length. In some embodiments, the oligonucleotide comprises one or more modified nucleotides. In some embodiments, the oligonucleotide comprises one or more C-5 modified pyrimidines. In some embodiments, the oligonucleotide comprises the sequence [(A-C-X-X)₇-A-C], wherein X is a BndU. In some embodiments, the oligonucleotide in the formulation has a concentration of from 5 μM to 100 μM or from 10 μM to 80 μM or from 20 μM to 60 μM or from 30 μM to 50 μM or about 75 μM or about 37 μM.

In some embodiments, the sample is dried at a constant temperature of from about 4° C. to about 8° C. In some embodiments, the sample is dried from about 4 hours to about 48 hours.

In some embodiments, the sample is diluted to a first dilution and a second dilution, wherein the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%. In some embodiments, the sample is diluted to a first dilution and a second dilution, wherein the first dilution is a dilution of the test sample of from 0.001% to 0.009% (or 0.001%, 0.002%, 0.003%, 0.004%, 0.005%, 0.006%, 0.007%, 0.008% or 0.009%) or is from 0.002% to 0.008%, or is from 0.003% to 0.007% or is about 0.005%; and the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%. In some embodiments, the sample is diluted to a first dilution and a second dilution, wherein the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the second dilution is a dilution of the test sample of from 5% to 39% (or is 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%. In some embodiments, the sample is diluted to a first dilution and a second dilution, wherein the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%; and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%. In some embodiments, the sample is diluted to a first dilution and a second dilution, wherein the first dilution is a dilution of the test sample of from 5% to 39% (or is 5%, 6%, 7%, 8%, 9%, 10%, 11%, 12%, 13%, 14%, 15%, 16%, 17%, 18%, 19%, 20, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.1% to 0.8%, or is from 0.2% to 0.75%, or is about 0.5%. In some embodiments, the sample is diluted to a first dilution and a second dilution, wherein the first dilution is a dilution of the test sample of from 5% to 39% (or is 5%, 6%, 7%, 8%, 9%, 10%, 11%, 12%, 13%, 14%, 1%, 1⁶%, 1⁷%, 1⁸%, 1⁹%, 20%, 21%, 22%, 23%, 24%, 25%, 26%, 27%, 28%, 29%, 30%, 31%, 32%, 33%, 34%, 35%, 36%, 37%, 38% or 39%), or is from 15% to 30%, or is from 15% to 25%, or is about 20%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008%, or is from 0.003% to 0.007%, or is about 0.005%. In some embodiments, the sample is diluted to a third dilution, wherein the third dilution is a dilution of the test sample selected from 5% to 39% (or is 5%, 6%, 7%, 8%, 9%, 10%, 11%, 12%, 13%, 14%, 15%, 16%, 17%, 18%, 1⁹%, 20, 21%, 22%, 23%, 24%, 25%, 26%, 27%, 28%, 29%, 30%, 31%, 32%, 33%, 34%, 35%, 36%, 37%, 38% or 39%), from 15% to 30%, from 15% to 25%, about 20%; from 0.01% to 1% (or 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%), from 0.1% to 0.8%, from 0.2% to 0.75%, about 0.5%; and from 0.001% to 0.009% (or 0.001%, 0.002%, 0.003%, 0.004%, 0.005%, 0.006%, 0.007%, 0.008% or 0.009%), or from 0.002% to 0.008%, from 0.003% to 0.007%, about 0.005%.

In some embodiments, the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%. In some embodiments, the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%. In some embodiments, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%, and the second dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%. In some embodiments, the first dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%. In some embodiments, the first dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%, and the second dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%. In some embodiments, the first dilution is a dilution of the test sample of from 0.5% to 5% (or is 0.5%, 0.6%, 0.7%, 0.8%, 0.9%, 1%, 1.2%, 1.5%, 1.7%, 2%, 2.2%, 2.5%, 2.7%, 3%, 3.2%, 3.5%, 3.7%, 4%, 4.2%, 4.5%, 4.7%, or 5%) or is from 0.5% to 4% or is from 1% to 3% or is about 2.5%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.6%, 0.7%, 0.8%, 0.9% or 1%) or is from 0.01% to 0.5% or is from 0.02% to 0.1% or is about 0.05%.

In some embodiments, compositions are provided. The composition comprises a dried sample and a formulation comprising a buffering agent, one or more salts, a chelating agent, a protease inhibitor, a non-ionic surfactant, and an oligonucleotide, wherein, the dried sample was derived from a liquid sample dried at a constant temperature of from about −20° C. to room temperature for at least four (4) hours.

In some embodiments, methods for detecting an analyte in a sample are provided. The methods comprise drying the sample at a constant temperature of from about −20° C. to room temperature for at least four (4) hours to create a dried sample, reconstituting the dried sample with a formulation comprising a buffering agent, one or more salts, a chelating agent, a protease inhibitor, a non-ionic surfactant, and an oligonucleotide, and detecting the analyte from the reconstituted sample.

In some embodiments, the detecting is performed with a protein binding reagent or a mass spectrometer.

In some embodiments, the protein binding reagent is selected from an aptamer or antibody.

In some embodiments, the detecting is performed in a multiplex assay. In some embodiments, the multiplex assay detects at least 2, 5, 10, 20, 30, 40, 50, 60, 70, 80, 90 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000, 10500, 11000, 12000, 13000, 14000, 15000, 16000, 17000, 18000, 19000 or 20000 analytes.

The foregoing and other objects, features, and advantages of the disclosure will become more apparent from the following detailed description, which proceeds with reference to the accompanying figures.

### BRIEF DESCRIPTION OF THE DRAWINGS

FIG. 1 illustrates the linear range of dilution group 1. The x-axis represents the concentration (%) of the DBS extract on a log 10 scale (from 20% down to 0) and the y-axis counts the number of analytes with a linear signal response at that dilution.

FIGS. 2A and 2B illustrate the linear range of dilution group 2 (FIG. 2A) and of dilution group 3 (FIG. 2B). The y-axis represents the number of analytes within their linear range. The x-axis represents the concentration (%) of the DBS extract on a log 10 scale.

FIGS. 3A-3C illustrate analytes with signals above background for dilution groups 1, 2, and 3, respectively.

FIGS. 4A and 4B illustrate the population Coefficient of Variation for all the analytes tested.

FIGS. 5A-5C illustrate concordance plots of the Median (n=3) relative fluorescents units (RFU) between a standard serum sample and the same sample after dying and extraction using water (5A) or PBS (5B) as the volume expander and PBS compared to water (5C).

FIGS. 6A and 6B illustrate the analyte recovery over different extraction times for two different sample collections.

FIGS. 7A-7C show the mean absolute error of signals of the multiplex assay performed on dried plasma at 4° C. (7A), room temperature (7B), −20° C. (7C), and temperature stressed compared to liquid plasma.

FIGS. 8A-8D compare the concordance between frozen plasma vs dried blood spots (8A), frozen serum vs dried blood spots (8B), frozen serum vs frozen plasma (8C), and two pooled DBS samples (8D).

FIG. 9 illustrates correlations of protein measurements between DBS and plasma/serum.

FIGS. 10A and 10B show significant correlations from paired observations mapped to a concordance plot. FIG. 10A shows correlations from paired observations and FIG. 10B shows correlations using an FDR of 5%.

FIG. 11 shows overlap in significant analytes between both methods shown in FIGS. 10A and 10B.

FIGS. 12A-12D show concordance of analytes with significant correlations between DBS and plasma.

FIGS. 13A-13D show significant correlations between DBS and plasma mapped on a serum vs plasma concordance plot.

FIG. 14 shows classification of analytes by biological function.

FIG. 15 shows a CDF Plot of 2,941,335 Random Correlations Between Plasma and Serum.

FIG. 16 shows the probability distribution of false positives based on Pearson cut-offs.

FIG. 17 shows an example of finding the weighted averages of false positives.

FIG. 18 shows a plot of significant correlations versus false correlations.

FIG. 19 shows a plot of the average FDR for a given Pearson cut-off.

FIG. 20 shows a plot of the number of significant correlations as a function of FDR.

FIG. 21 illustrates certain exemplary 5-position modified uridines and cytidines that may be incorporated into aptamers.

FIG. 22 illustrates certain exemplary modifications that may be present at the 5-position of uridine. The chemical structure of the C-5 modification includes the exemplary amide linkage that links the modification to the 5-position of uridine. The 5-position moieties shown include two phenyl groups covalently attached to one another. The 5-position moieties shown include a phenylbenzyl moiety (e.g., BPE, PBnd, DBM), a 4-phenoxybenzyl moiety (e.g., POP), a diphenylpropyl moiety (e.g., DPP), a benzhydryl moiety (e.g., BH).

FIG. 23 illustrates certain exemplary modifications that may be present at the 5-position of cytidine. The chemical structure of the C-5 modification includes the exemplary amide linkage that links the modification to the 5-position of cytidine. The 5-position moieties shown include two phenyl groups covalently attached to one another. The 5-position moieties shown include a phenylbenzyl moiety (e.g., BPE, PBnd, DBM), a 4-phenoxybenzyl moiety (e.g., POP), a diphenylpropyl moiety (e.g., DPP), a benzhydryl moiety (e.g., BH).

FIG. 24 illustrates certain exemplary modifications that may be present at the 5-position of uridine. The chemical structure of the C-5 modification includes the exemplary amide linkage that links the modification to the 5-position of the uridine. The 5-position moieties shown include a benzyl moiety (e.g., Bn, PE and a PP), a naphthyl moiety (e.g., Nap, 2Nap, NE), a butyl moiety (e.g, iBu), a fluorobenzyl moiety (e.g., FBn), a tyrosyl moiety (e.g., a Tyr), a 3,4-methylenedioxy benzyl (e.g., MBn), a morpholino moiety (e.g., MOE), a benzofuranyl moiety (e.g., BF), an indole moiety (e.g, Trp) and a hydroxypropyl moiety (e.g., Thr).

FIG. 25 illustrates certain exemplary modifications that may be present at the 5-position of cytidine. The chemical structure of the C-5 modification includes the exemplary amide linkage that links the modification to the 5-position of the cytidine. The 5-position moieties shown include a benzyl moiety (e.g., Bn, PE and a PP), a naphthyl moiety (e.g., Nap, 2Nap, NE, and 2NE) and a tyrosyl moiety (e.g., a Tyr).

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include the plural unless context clearly indicates otherwise, and may be used interchangeably with “at least one” and “one or more.” “Comprising A or B” means including A, or B, or A and B. It is further to be understood that all base sizes or amino acid sizes, and all molecular weight or molecular mass values, given for nucleic acids or polypeptides are approximate, and are provided for description.

Further, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements may include other elements not expressly listed.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

As used herein, the term “nucleotide” refers to a ribonucleotide or a deoxyribonucleotide, or a modified form thereof, as well as an analog thereof. Nucleotides include species that include purines (e.g., adenine, hypoxanthine, guanine, and their derivatives and analogs) as well as pyrimidines (e.g., cytosine, uracil, thymine, and their derivatives and analogs). As used herein, the term “cytidine” is used generically to refer to a ribonucleotide, deoxyribonucleotide, or modified ribonucleotide comprising a cytosine base, unless specifically indicated otherwise. The term “cytidine” includes 2′-modified cytidines, such as 2′-fluoro, 2′-methoxy, etc. Similarly, the term “modified cytidine” or a specific modified cytidine also refers to a ribonucleotide, deoxyribonucleotide, or modified ribonucleotide (such as 2′-fluoro, 2′-methoxy, etc.) comprising the modified cytosine base, unless specifically indicated otherwise. The term “uridine” is used generically to refer to a ribonucleotide, deoxyribonucleotide, or modified ribonucleotide comprising a uracil base, unless specifically indicated otherwise. The term “uridine” includes 2′-modified uridines, such as 2′-fluoro, 2′-methoxy, etc. Similarly, the term “modified uridine” or a specific modified uridine also refers to a ribonucleotide, deoxyribonucleotide, or modified ribonucleotide (such as 2′-fluoro, 2′-methoxy, etc.) comprising the modified uracil base, unless specifically indicated otherwise.

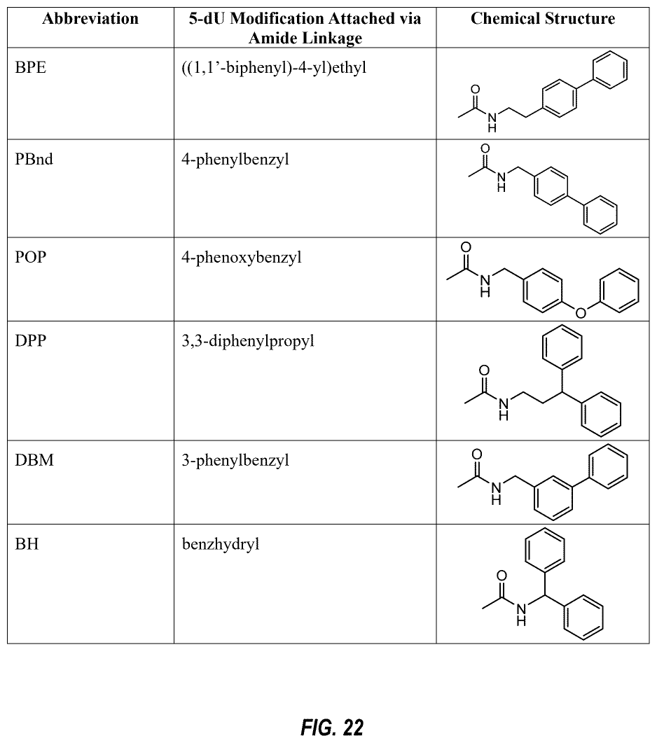

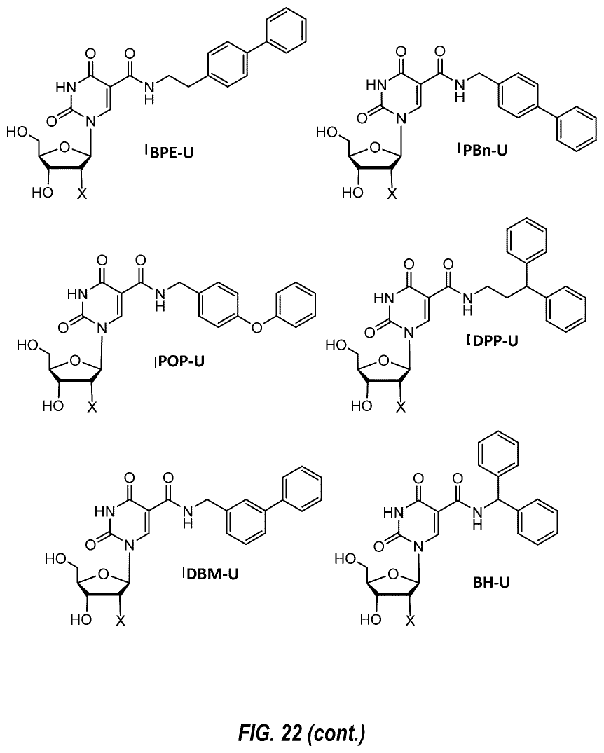

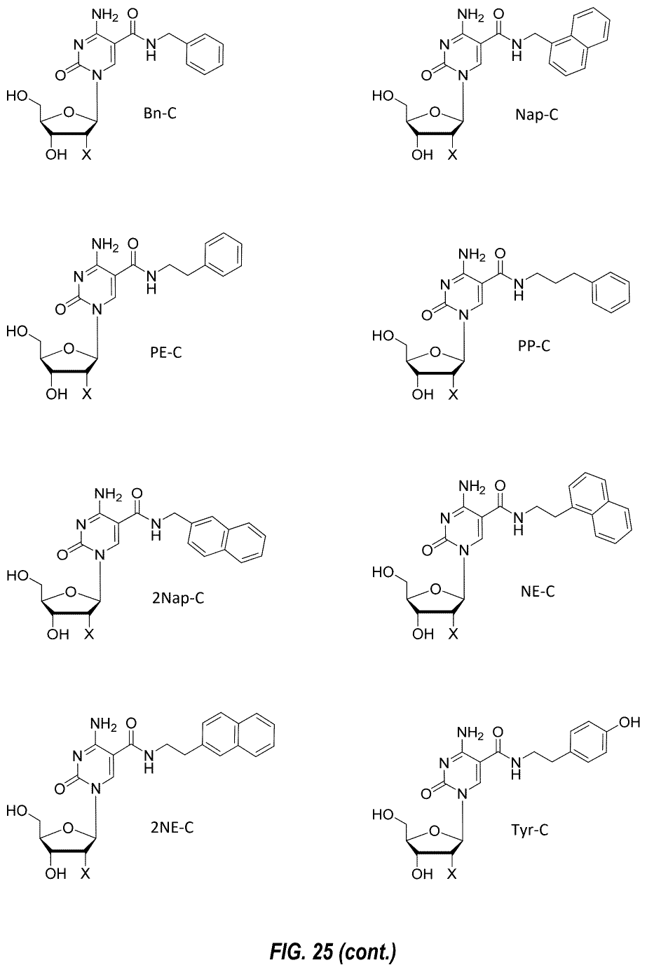

As used herein, the term “C-5 modified carboxamidecytidine” or “cytidine-5-carboxamide” or “5-position modified cytidine” or “C-5 modified cytidine” refers to a cytidine with a carboxyamide (—C(O)NH—) modification at the C-5 position of the cytidine including, but not limited to, those moieties (R^{X1}) illustrated herein. Exemplary C-5 modified carboxamidecytidines include, but are not limited to, 5-(N-benzylcarboxamide)-2′-deoxycytidine (referred to as “BndC” and shown in FIG. 25); 5-(N-2-phenylethylcarboxamide)-2′-deoxycytidine (referred to as “PEdC” and shown in FIG. 25); 5-(N-3-phenylpropylcarboxamide)-2′-deoxycytidine (referred to as “PPdC” and shown in FIG. 25); 5-(N-1-naphthylmethylcarboxamide)-2′-deoxycytidine (referred to as “NapdC” and shown in FIG. 22); 5-(N-2-naphthylmethylcarboxamide)-2′-deoxycytidine (referred to as “2NapdC” and shown in FIG. 25); 5-(N-1-naphthyl-2-ethylcarboxamide)-2′-deoxycytidine (referred to as “NEdC” and shown in FIG. 25); 5-(N-2-naphthyl-2-ethylcarboxamide)-2′-deoxycytidine (referred to as “2NEdC” and shown in FIG. 25); and 5-(N-tyrosylcarboxyamide)-2′-deoxycytidine (referred to as TyrdC and shown in FIG. 25). In some embodiments, the C5-modified cytidines, e.g., in their triphosphate form, are capable of being incorporated into an oligonucleotide by a polymerase (e.g., KOD DNA polymerase).

Chemical modifications of the C-5 modified cytidines described herein can also be combined with, singly or in any combination, 2′-position sugar modifications, modifications at exocyclic amines, and substitution of 4-thiocytidine and the like.

As used herein, the term “C-5 modified carboxamidecytosine” or “cytosine-5-carboxamide” or “5-position modified cytosine” or “C-5 modified cytosine” refers to a cytosine base with a carboxyamide (—C(O)NH—) modification at the C-5 position of the cytosine including, but not limited to, those moieties (R^{X1}) illustrated herein. Exemplary C-5 modified carboxamidecytosines include, but are not limited to, the modified cytidines shown in FIG. 25.

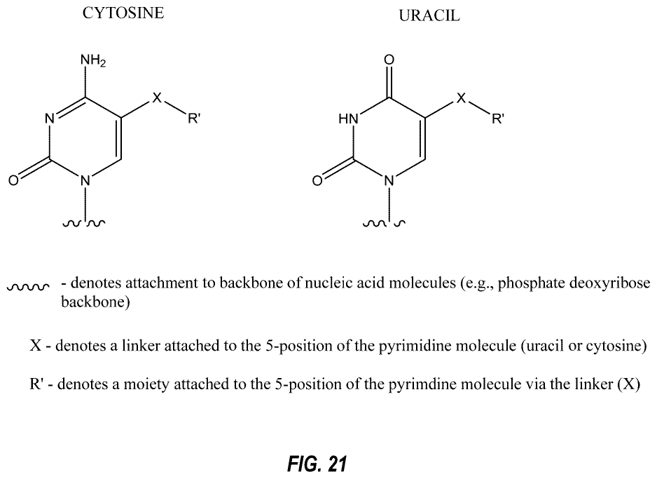

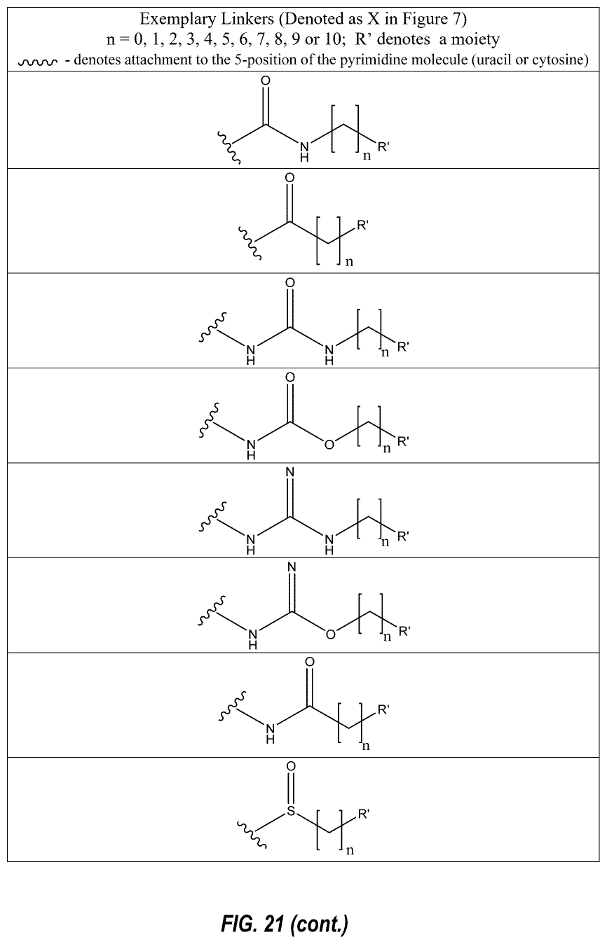

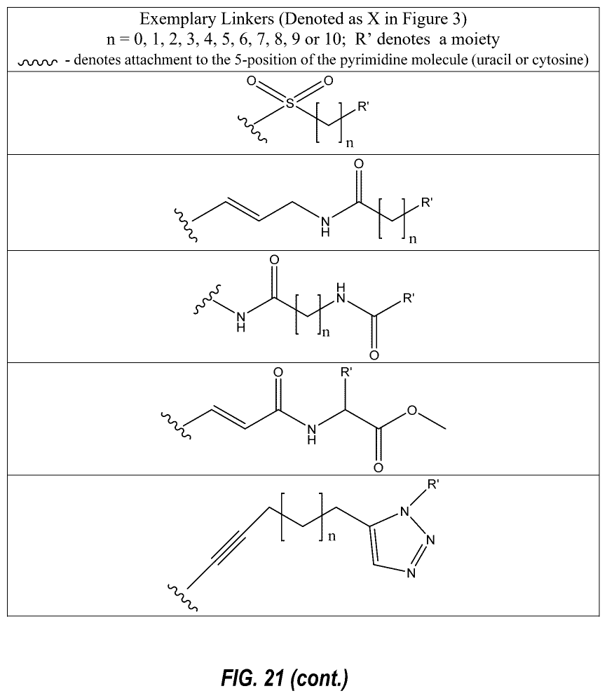

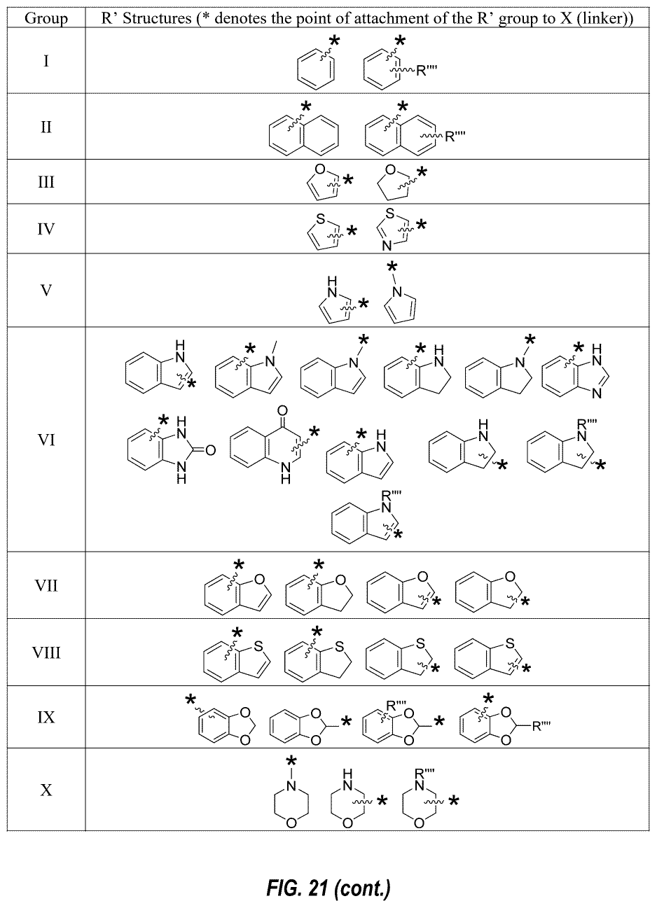

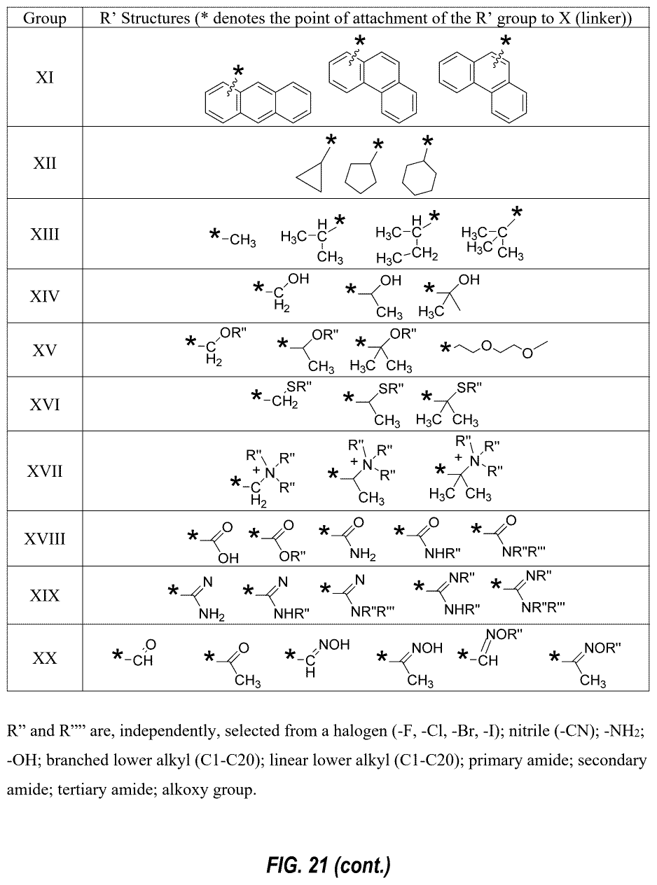

As used herein, the term “C-5 modified uridine” or “5-position modified uridine” refers to a uridine (typically a deoxyuridine) with a carboxyamide (—C(O)NH—) modification at the C-5 position of the uridine, e.g., as shown in FIG. 21. In some embodiments, the C5-modified uridines, e.g., in their triphosphate form, are capable of being incorporated into an oligonucleotide by a polymerase (e.g., KOD DNA polymerase). Nonlimiting exemplary 5-position modified uridines include:
5-(N-benzylcarboxyamide)-2′-deoxyuridine (BndU),
5-(N-benzylcarboxyamide)-2′-O-methyluridine,
5-(N-benzylcarboxyamide)-2′-fluorouridine,
5-(N-phenethylcarboxyamide)-2′-deoxyuridine (PEdU),
5-(N-thiophenylmethylcarboxyamide)-2′-deoxyuridine (ThdU),
5-(N-isobutylcarboxyamide)-2′-deoxyuridine (iBudU),
5-(N-tyrosylcarboxyamide)-2′-deoxyuridine (TyrdU),
5-(N-3,4-methylenedioxybenzylcarboxyamide)-2′-deoxyuridine (MBndU),
5-(N-4-fluorobenzylcarboxyamide)-2′-deoxyuridine (FBndU),
5-(N-3-phenylpropylcarboxyamide)-2′-deoxyuridine (PPdU),
5-(N-imidizolylethylcarboxyamide)-2′-deoxyuridine (ImdU),
5-(N-isobutylcarboxyamide)-2′-O-methyluridine,
5-(N-isobutylcarboxyamide)-2′-fluorouridine,
5-(N-tryptaminocarboxyamide)-2′-deoxyuridine (TrpdU),
5-(N—R-threoninylcarboxyamide)-2′-deoxyuridine (ThrdU),
5-(N-tryptaminocarboxyamide)-2′-O-methyluridine,
5-(N-tryptaminocarboxyamide)-2′-fluorouridine,
5-(N-[1-(3-trimethylamonium) propyl]carboxyamide)-2′-deoxyuridine chloride,
5-(N-naphthylmethylcarboxyamide)-2′-deoxyuridine (NapdU),
5-(N-naphthylmethylcarboxyamide)-2′-O-methyluridine,
5-(N-naphthylmethylcarboxyamide)-2′-fluorouridine,
5-(N-[1-(2,3-dihydroxypropyl)]carboxyamide)-2′-deoxyuridine),
5-(N-2-naphthylmethylcarboxyamide)-2′-deoxyuridine (2NapdU),
5-(N-2-naphthylmethylcarboxyamide)-2′-O-methyluridine,
5-(N-2-naphthylmethylcarboxyamide)-2′-fluorouridine,
5-(N-1-naphthylethylcarboxyamide)-2′-deoxyuridine (NEdU),
5-(N-1-naphthylethylcarboxyamide)-2′-O-methyluridine,
5-(N-1-naphthylethylcarboxyamide)-2′-fluorouridine,
5-(N-2-naphthylethylcarboxyamide)-2′-deoxyuridine (2NEdU),
5-(N-2-naphthylethylcarboxyamide)-2′-O-methyluridine,
5-(N-2-naphthylethylcarboxyamide)-2′-fluorouridine,
5-(N-3-benzofuranylethylcarboxyamide)-2′-deoxyuridine (BFdU),
5-(N-3-benzofuranylethylcarboxyamide)-2′-O-methyluridine,
5-(N-3-benzofuranylethylcarboxyamide)-2′-fluorouridine,
5-(N-3-benzothiophenylethylcarboxyamide)-2′-deoxyuridine (BTdU),
5-(N-3-benzothiophenylethylcarboxyamide)-2′-O-methyluridine, and
5-(N-3-benzothiophenylethylcarboxyamide)-2′-fluorouridine.

Addition C-5 modifications may be found in WO/2022/221241.

As used herein, the terms “modify,” “modified,” “modification,” and any variations thereof, when used in reference to an oligonucleotide, means that at least one of the four constituent nucleotide bases (i.e., A, G, T/U, and C) of the oligonucleotide is an analog or ester of a naturally occurring nucleotide. In some embodiments, the modified nucleotide confers nuclease resistance to the oligonucleotide. Additional modifications can include backbone modifications, methylations, unusual base-pairing combinations such as the isobases isocytidine and isoguanidine, and the like. Modifications can also include 3′ and 5′ modifications, such as capping. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present on the sugar of a nucleotide may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, polyethylene glycol (PEG) polymers in one embodiment ranging from about 10 to about 80 kDa, PEG polymers in another embodiment ranging from about 20 to about 60 kDa, or other hydrophilic or hydrophobic biological or synthetic polymers.

As used herein, “nucleic acid,” “oligonucleotide,” and “polynucleotide” are used interchangeably to refer to a polymer of nucleotides and include DNA, RNA, DNA/RNA hybrids and modifications of these kinds of nucleic acids, oligonucleotides and polynucleotides, wherein the attachment of various entities or moieties to the nucleotide units at any position are included. The terms “polynucleotide,” “oligonucleotide,” and “nucleic acid” include double- or single-stranded molecules as well as triple-helical molecules. Nucleic acid, oligonucleotide, and polynucleotide are broader terms than the term aptamer and, thus, the terms nucleic acid, oligonucleotide, and polynucleotide include polymers of nucleotides that are aptamers but the terms nucleic acid, oligonucleotide, and polynucleotide are not limited to aptamers.

Polynucleotides can also contain analogous forms of ribose or deoxyribose sugars that are generally known in the art, including 2′-O-methyl, 2′-O-allyl, 2′-O-ethyl, 2′-O-propyl, 2′-O—CH₂CH₂OCH₃, 2′-fluoro, 2′-NH₂ or 2′-azido, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted herein, one or more phosphodiester linkages may be replaced by alternative linking groups. These alternative linking groups include embodiments wherein phosphate is replaced by P(O)S (“thioate”), P(S)S (“dithioate”), (O)NR^{X}₂ (“amidate”), P(O) R^{X}, P(O)OR^{X}′, CO or CH₂ (“formacetal”), in which each R^{X} or R^{X}′ are independently H or substituted or unsubstituted alkyl (C1-C20) optionally containing an ether (—O—) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

Polynucleotides can also contain analogous forms of carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside.

If present, a modification to the nucleotide structure can be imparted before or after assembly of a polymer. A sequence of nucleotides can be interrupted by non-nucleotide components. A polynucleotide can be further modified after polymerization, such as by conjugation with a labeling component.

As used herein, the term “at least one nucleotide” when referring to modifications of a nucleic acid, refers to one, several, or all nucleotides in the nucleic acid, indicating that any or all occurrences of any or all of A, C, T, G or U in a nucleic acid may be modified or not.

The term “antibody” refers to full-length antibodies of any species and fragments and derivatives of such antibodies that retain the ability to bind to antigen, including Fab fragments, F(ab′)₂ fragments, single chain antibodies, Fv fragments, and single chain Fv fragments. The term “antibody” also includes synthetically-derived antibodies, such as phage display-derived antibodies and fragments, affybodies and nanobodies.

As used herein, “nucleic acid ligand,” “aptamer,” “SOMAmer,” “modified aptamer,” and “clone” are used interchangeably to refer to a non-naturally occurring nucleic acid that has a desirable action on a target molecule. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule. In one embodiment, the action is specific binding affinity for a target molecule, such target molecule being a three dimensional chemical structure other than a polynucleotide that binds to the aptamer through a mechanism which is independent of Watson/Crick base pairing or triple helix formation, wherein the aptamer is not a nucleic acid having the known physiological function of being bound by the target molecule. Aptamers to a given target include nucleic acids that are identified from a candidate mixture of nucleic acids, where the aptamer is a ligand of the target, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target generally with a much higher degree of affinity than it binds to other, non-target, components in a mixture or sample. An “aptamer,” “SOMAmer,” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In some embodiments, the aptamers are prepared using a SELEX process as described herein, or known in the art.

As used herein, a “SOMAmer” or Slow Off-Rate Modified Aptamer refers to an aptamer having improved off-rate characteristics. SOMAmers can be generated using the improved SELEX methods described in U.S. Pat. No. 7,947,447, entitled “Method for Generating Aptamers with Improved Off-Rates.”

As used herein, an aptamer comprising two different types of 5-position modified pyrimidines or C-5 modified pyrimidines may be referred to as “dual modified aptamers”, aptamers having “two modified bases”, aptamers having “two base modifications” or “two bases modified”, aptamer having “double modified bases”, all of which may be used interchangeably. A library of aptamers or aptamer library may also use the same terminology. Thus, in some embodiments, an aptamer comprises two different 5-position modified pyrimidines wherein the two different 5-position modified pyrimidines are selected from a NapdC and a NapdU, a NapdC and a PPdU, a NapdC and a MOEdU, a NapdC and a TyrdU, a NapdC and a ThrdU, a PPdC and a PPdU, a PPdC and a NapdU, a PPdC and a MOEdU, a PPdC and a TyrdU, a PPdC and a ThrdU, a NapdC and a 2NapdU, a NapdC and a TrpdU, a 2NapdC and a NapdU, and 2NapdC and a 2NapdU, a 2NapdC and a PPdU, a 2NapdC and a TrpdU, a 2NapdC and a TyrdU, a PPdC and a 2NapdU, a PPdC and a TrpdU, a PPdC and a TyrdU, a TyrdC and a TyrdU, a TrydC and a 2NapdU, a TyrdC and a PPdU, a TyrdC and a TrpdU, a TyrdC and a TyrdU, and a TyrdC and a TyrdU. In some embodiments, an aptamer comprises at least one modified uridine and/or thymidine and at least one modified cytidine, wherein the at least one modified uridine and/or thymidine is modified at the 5-position with a moiety selected from a naphthyl moiety, a benzyl moiety, a fluorobenzyl moiety, a tyrosyl moiety, an indole moiety a morpholino moiety, an isobutyl moiety, a 3,4-methylenedioxy benzyl moiety, a benzothiophenyl moiety, and a benzofuranyl moiety, and wherein the at least one modified cytidine is modified at the 5-position with a moiety selected from a naphthyl moiety, a tyrosyl moiety, and a benzyl moiety. In certain embodiments, the moiety is covalently linked to the 5-position of the base via a linker comprising a group selected from an amide linker, a carbonyl linker, a propynyl linker, an alkyne linker, an ester linker, a urea linker, a carbamate linker, a guanidine linker, an amidine linker, a sulfoxide linker, and a sulfone linker. See FIG. 21 for further examples of exemplary linkers that may be used to covalently link a moiety to the 5-position of a pyrimidine.

As used herein, a “hydrophobic group” and “hydrophobic moiety” are used interchangeably herein and refer to any group or moiety that is uncharged, a majority of the atoms of the group or moiety are hydrogen and carbon, the group or moiety has a small dipole and/or the group or moiety tends to repel from water. These groups or moieties may comprise an aromatic hydrocarbon or a planar aromatic hydrocarbon. Methods for determining the hydrophobicity or whether molecule (or group or moiety) is hydrophobic are well known in the art and include empirically derived methods, as well as calculation methods. Exemplary methods are described in Zhu Chongqin et al. (2016) Characterizing hydrophobicity of amino acid side chains in a protein environment via measuring contact angle of a water nanodroplet on planar peptide network. Proc. Natl. Acad. Sci., 113(46) pgs. 12946-12951. As disclosed herein, exemplary hydrophobic moieties included, but are not limited to, Groups I, II, III, IV, V, VII, VIII, IX, XI, XII, XIII, XV and XVI of FIG. 21. Further exemplary hydrophobic moieties include those of FIG. 25 (e.g., Bn, Nap, PE, PP, iBu, 2Nap, Try, NE, MBn, BF, BT, Trp).

As used herein, an aptamer comprising a single type of 5-position modified pyrimidine or C-5 modified pyrimidine may be referred to as “single modified aptamers”, aptamers having a “single modified base”, aptamers having a “single base modification” or “single bases modified”, all of which may be used interchangeably. A library of aptamers or aptamer library may also use the same terminology. As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

In certain embodiments, an aptamer comprises a first 5-position modified pyrimidine and a second 5-position modified pyrimidine, wherein the first 5-position modified pyrimidine comprises a tryosyl moiety at the 5-position of the first 5-position modified pyrimidine, and the second 5-position modified pyrimidine comprises a naphthyl moiety or benzyl moiety at the 5-position at the second 5-position modified pyrimidine. In a related embodiment the first 5-position modified pyrimidine is a uracil. In a related embodiment, the second 5-position modified pyrimidine is a cytosine. In a related embodiment, at least 10%, 15%, 20%, 25%, 30%, 35%, 40%, 45%, 50%, 55%, 60%, 65%, 70%, 75%, 80%, 85%, 90%, 95%, or 100% of the uracils of the aptamer are modified at the 5-position. In a related embodiment, at least 10%, 15%, 20%, 25%, 30%, 35%, 40%, 45%, 50%, 55% 60%, 65%, 70%, 75%, 80%, 85%, 90%, 95%, or 100% of the cytosine of the aptamer are modified at the 5-position.

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well-known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

As used herein, “Hybridization,” “hybridizing,” “binding” and like terms, in the context of nucleotide sequences, can be used interchangeably herein. The ability of two nucleotide sequences to hybridize with each other is based on the degree of complementarity of the two sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency is achieved by elevating the temperature, increasing the ratio of co-solvents, lowering the salt concentration, and the like. Hybridization of complementary Watson/Crick base pairs of probes on the microarray and of the target material is generally preferred, but non-Watson/Crick base pairing during hybridization can also occur.

Conventional hybridization solutions and processes for hybridization are described in J. Sambrook, Molecular Cloning: A Laboratory Manual, (supra), incorporated herein by reference. Conditions for hybridization typically include (1) high ionic strength solution, (2) at a controlled temperature, and (3) in the presence of carrier DNA and surfactants and chelators of divalent cations, all of which are known in the art.

As used herein, “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

As used herein, “array” includes any one, two or three-dimensional arrangement of addressable regions bearing a particular chemical moiety or moieties (for example, biopolymers such peptide nucleic acid molecules, peptides or polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm or even less than 10 cm. For example, features may have widths (that is, diameter, for a round spot) in the range of from about 10 m to about 1.0 cm. In other embodiments each feature may have a width in the range of about 1.0 m to about 1.0 mm, such as from about 5.0 m to about 500 m, and including from about 10 m to about 200 m. Non-round features may have area ranges equivalent to that of circular features with the foregoing width (diameter) ranges. A given feature is made up of chemical moieties, e.g., peptide nucleic acid molecules, peptides, nucleic acids, that bind to (e.g., hybridize to) the target molecule (e.g., target nucleic acid or aptamer), such that a given feature corresponds to a particular target.

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other. In some embodiments, the target is an oligonucleotide or aptamer. In some embodiments, the probe is a peptide nucleic acid molecule, peptide, protein, oligonucleotide or aptamer.

The term “biological sample”, “sample”, and “test sample” are used interchangeably herein to refer to any material, biological fluid, tissue, or cell obtained or otherwise derived from an individual, and environmental, animal, or food sample. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the preceding.

For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

A collection device as used herein may be any suitable absorbent device that can absorb the sample to be dried. Non-limiting examples include filter paper such as Whatman®, Guthrie cards, MITRA® microsampling devices, Capitainer® microsampling devices, TAPII microsampling devices, Tasso devices, dried fluid cards, and similar devices.

As used herein, “target protein level,” “analyte level” and “level” or “target protein value,” “analyte value” and “value” refer to a measurement that is made using any analytical method for detecting the analyte (such as a target protein) in a biological sample and that indicates the presence, absence, absolute amount or concentration, relative amount or concentration, titer, level, expression level, ratio of measured levels, or the like, of, for, or corresponding to the analyte in the biological sample. The exact nature of the “level” or “value” depends on the specific design and components of the particular analytical method employed to detect the analyte.

As used herein, a “capture agent” or “capture reagent” refers to a molecule that is capable of binding specifically to an analyte, such as a biomarker, protein and/or peptide. A “target protein capture reagent” refers to a molecule that is capable of binding specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents. In some embodiments, a capture reagent is selected from an aptamer and an antibody.

A “control level” or “control value” of a target molecule refers to the level of the target molecule in the same sample type from an individual that does not have the disease or condition, or from an individual that is not suspected or at risk of having the disease or condition, or from an individual that has a non-progressive form of the disease or condition.

Further, a “control level” or “control value” may refer to a reference based on the average or what is considered within normal or healthy parameters. A “control level” or “control value” may also refer to a reference level taken at a previous time and that is used to compare to a later measured or detected level of a target. For example, the level of a target may be detected at time point A, and then detected at time point B, where time point B is after time point A. In a more specific example, time point A may be considered time zero (0) or day zero (0) and time point B may be minutes (e.g., 10, 20, 30, 40, 50, 60 minutes after time point A), hours (e.g, 1, 2, 3, 4, 5, 6, 7, 8, 9, 10, 11, 12, 13, 14, 15, 16, 17, 18, 19, 20, 21, 22, 23 or 24 hours after time point A), days (e.g, 1, 2, 3, 4, 5, 6 or 7 days after time point A), weeks (e.g., 1, 2, 3 or 4 weeks after time point A), months (e.g., 1, 2, 3, 4, 5, 6, 7, 8, 9, 10, 11 or 12 months after time point A) and even years (e.g., 1, 2, 3, 4, 5, 6, 7, 8, 9, 10, 11, 12, 13, 14, 15, 20, 25, 30, 35, 40, 45, 50, 55 or 60 years after time point A) after time point A. A “control level” of a target molecule need not be determined each time the present methods are carried out, and may be a previously determined level that is used as a reference or threshold to determine whether the level in a particular sample is higher or lower than a normal level.

“Correspondence correlation” or “concordance correlation coefficient” measures the agreement between two continuous variables X and Y (e.g., predicted, estimated or determined, and actual). The “correspondence correlation” evaluates the degree to which pairs fall on the 450 line, and contains measurements of accuracy and precision (or the “Lin's Condordance”). Additional information may be found in Lin, Biometrics, Vol. 45, No. 1 (March, 1989), 255-268, which is hereby incorporated by reference. Other methods for determining correlation that may be used herein includes, but are not limited to, Pearson correlation coefficient, the paired t-test, least squares analysis of slope (=1) and intercept (=0), the coefficient of variation and the intraclass correlation coefficient. In certain embodiments, the correspondence correlation is determined by the method selected from Lin's Concordance, Pearson correlation coefficient, the paired t-test, least squares analysis of slope (=1) and intercept (=0), the coefficient of variation and the intraclass correlation coefficient.

As used herein, “detecting” or “determining” includes the use of both the instrument used to observe and record a signal corresponding to an analyte level and the material/s required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Dilution”, “dilution series” and variations thereof encompass several different types of dilutions, including, but not limited to, step dilutions, serial dilutions and combinations thereof. By way of example for a step dilution, if the dilution factor is 1000 (1:1000 dilution), the user may first perform a 1:10 dilution (dilution factor of 10) followed by a 1:100 dilution (dilution factor of 100) using 1 part solute from the 1:10 dilution and 99 parts of diluent, thus resulting in a dilution factor of 1000 or 1:1000 dilution of the solute. A serial dilution includes a succession of step dilutions, each having the same dilution factor, where the diluted material from the previous step is used to make the subsequent dilution. By way of example for a serial dilution, to make a 5-point 1:2 serial dilution, entails using 1 part solute and combining with 1 part diluent to make the first dilution (1^{st} point of the 5-point) in the dilution series, followed by 1 part solute from the first dilution and combining with 1 part diluent to make the second dilution (2^{nd} point of the 5-point) of the serial dilution series, so on and so forth until you reach the fifth successive serial dilution.

“Dilution factor” refers to the ratio of the parts of solute to parts of diluent. For example, a dilution factor of 2 means a 1:2 dilution where there are 1 part solute and 1 part diluent for a total of 2 parts; and a dilution factor of 10 means a 1:10 dilution where there are 1 part solute and 9 parts diluent for a total of 10 parts.

“Target”, “target molecule”, and “analyte” are used interchangeably herein to refer to any molecule of interest that may be present in a sample. The term includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” refers to a set of copies of one type or species of molecule or multi-molecular structure. “Target molecules”, “targets”, and “analytes” refer to more than one type or species of molecule or multi-molecular structure. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

As used herein, “test sample” means a material, solution, or mixture that comprises or is derived from a biological sample. In some embodiments, a test sample is generated from a biological sample. In some embodiments, a test sample is generated from a biological sample or a solution comprising a biological sample by performing a buffer exchange on the biological sample or solution comprising the biological sample. As used herein, an “adjusted test sample” is a test sample to which an adjustment has been made such as a change in total protein concentration.

The phrase “oligonucleotide bound to a surface of a solid support” or “probe bound to a solid support” or a “target bound to a solid support” refers to a peptide nucleic acid molecules, oligonucleotide, aptamer, e.g., PNA (peptide nucleic acid), LNA (locked nucleic acid) or UNA (unlocked nucleic acid) molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide or target elements employed herein are present on a surface of the same planar support, e.g., in the form of an array. It should be understood that the terms “probe” and “target” are relative terms and that a molecule considered as a probe in certain assays may function as a target in other assays. Immobilization of oligonucleotides on a substrate or surface can be accomplished by well-known techniques, commonly available in the literature. See for example A. C. Pease, et al., Proc. Nat. Acad. Sci, USA, 91:5022-5026 (1994); Z. Guo, et al., Nucleic Acids Res, 22, 5456-65 (1994); and M. Schena, et al., Science, 270, 467-70 (1995), each incorporated by reference herein.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in Southern, E. M., Maskos, U. and Elder, J. K., Genomics, 13, 1007-1017, 1992. In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

### Multiplex Assay

Multiplexed aptamer assays in solution-based target interaction and separation steps are described, e.g. in U.S. Pat. Nos. 7,855,054 and 7,964,356 and PCT Application PCT/US2013/044792. In one embodiment, a multiplex assay is described herein at Example 1.

In a multiplex assay format where multiple target proteins are being measured by multiple capture reagents, the natural variation in the abundance of the different target proteins can limit the ability of certain capture reagents to measure certain target proteins (e.g., high abundance target proteins may saturate the assay and prevent or reduce the ability of the assay to measure low abundance target proteins). To address this variation in the biological sample, the aptamer reagents may be separated into at least two different groups (Capture Reagents for DIL1 and Capture Reagents for DIL2), preferably three different groups (A3—Capture Reagents for DIL1; A2—Capture Reagents for DIL2 and A1—Capture Reagents for DIL3), based on the abundance of their respective protein target in the biological sample. Each of the capture reagent groups, A1, A2 and A3 each have a different set of aptamers, with the aptamers having specific affinity for a target protein. The biological sample is diluted into two (Dilution 1 or DIL1 and Dilution 2 or DIL2), preferably three, different dilution groups (Dilution 1 or DIL1; Dilution 2 or DIL2 and Dilution 3 or DIL3) to create separate test samples based on relative concentrations of the protein targets to be detected by their capture reagents. Thus, the biological sample is diluted into high and low, or high, medium and low abundant target protein dilution groups, where the least abundant protein targets are measured in the least diluted group, and the most abundant protein targets are measured in the greatest diluted group. The capture reagents for their respective dilution groups are incubated together (e.g., the A3 set of aptamers are incubated with the test sample of Dilution 1 or DIL1; the A2 set of aptamers are incubated with the test sample of Dilution 2 or DIL2 and the A1 set of aptamers are incubated with the test sample of Dilution 3 or DIL3). The total number of aptamers for A1, A2 and A3 may be 4,000; 4,500; 5,000 or more aptamers. In some embodiments, dilution group 1 may be 2.5%, dilution group 2 may be 0.05%, and dilution group 3 may be 0.005%. Other dilutions for the 3 dilution groups may also be used.

The present disclosure describes methods for the preparation of dried biologic matrices for detection of target molecules in a multiplex assay and to methods of detecting target molecules from dried biologic matrices in a multiplex assay.

The biologic matrix may be prepared for use in a multiplex assay as follows. The biologic matrix may be collected from a subject according to standard procedures such as a blood draw, fluid collection, biopsy collection, etc. Once collected, the biologic matrix may be spotted or adsorbed onto a collection device and dried.

### Drying Temperature

In some embodiments, the drying temperature may be from about −20° C. to about room temperature (about 20-24° C.). In some embodiments, the drying temperature may be about −20° C., about −15° C., about −10° C., about −5° C., about 0° C., about 2° C., about 4° C., about 6° C., about 8° C., about 10° C., about 12° C., about 14° C., about 16° C., about 20° C., about 22° C., or about 24° C.

### Drying Time

In some embodiments, the drying time may be about 1 hour to about 10 days. In some embodiments, the trying time may be about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 40 hours, about 44 hours, about 48 hours, about 52 hours, about 56 hours, about 60 hours, about 64 hours, about 68 hours, about 72 hours, about 4 days, about 5 days, about 6 days, about 7 days, about 8 days, about 9 days, or about 10 days. In some embodiments, the drying time may be at least 1 hour to at least 10 days. In some embodiments, the trying time may be at least 1 hour, at least 2 hours, at least 3 hours, at least 4 hours, at least 5 hours, at least 6 hours, at least 7 hours, at least 8 hours, at least 9 hours, at least 10 hours, at least 12 hours, at least 14 hours, at least 16 hours, at least 18 hours, at least 20 hours, at least 22 hours, at least 24 hours, at least 26 hours, at least 28 hours, at least 30 hours, at least 32 hours, at least 34 hours, at least 36 hours, at least 40 hours, at least 44 hours, at least 48 hours, at least 52 hours, at least 56 hours, at least 60 hours, at least 64 hours, at least 68 hours, at least 72 hours, at least 4 days, at least 5 days, at least 6 days, at least 7 days, at least 8 days, at least 9 days, or at least 10 days. In some embodiments, the drying time may be at a constant temperature until the sample is dried prior to any temperature fluctuation. In some embodiments, temperature fluctuation may occur with shipping and handling. In some embodiments, the dried sample is suitable for a multiplex assay when the drying temperature is constant until the sample is dried.

### Extraction Time

In some embodiments, the target molecules may be extracted from the collection device in a formulation for at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 70 minutes, at least 80 minutes, at least 90 minutes, at least 100 minutes, at least 110 minutes, at least 120 minutes, at least 130 minutes, at least 140 minutes, at least 150 minutes, at least 160 minutes, at least 170 minutes, at least 180 minutes, at least 190 minutes, or at least 200 minutes.

### Extraction Formulation

In some embodiments, the extraction formulation may comprise a buffering agent, one or more salts, a chelating agent, a protease inhibitor, a non-ionic surfactant, and an oligonucleotide. In some embodiments, the extraction formulation may comprise 20% PBS in addition to a buffering agent, one or more salts, a chelating agent, a protease inhibitor, a non-ionic surfactant, and an oligonucleotide. In some embodiments, the one or more salts are each independently selected from a sodium salt, a potassium salt and a magnesium salt.

In some embodiments, the one or more salts comprise a sodium salt, a potassium salt and a magnesium salt. In some embodiments, the sodium salt is NaCl, the potassium salt is KCl and the magnesium salt is MgCl2. In some embodiments, NaCl in the formulation is at a concentration of from about 10 mM to about 500 mM, or from about 50 mM to about 250 mM, or from about 100 mM to about 200 mM, or from about 75-125 mM, or about 100 mM. In some embodiments, the KCl in the formulation is at a concentration of from about 0.5 mM to about 30 mM, or from about 1 mM to about 20 mM, or from about 2 mM to about 15 mM, or from about 4 mM to about 10 mM, or about 5 mM. In some embodiments, the MgCl2 in the formulation is at a concentration of from about 0.5 mM to about 30 mM, or from about 1 mM to about 20 mM, or from about 2 mM to about 15 mM, or from about 4 mM to about 10 mM, or about 8 mM.

In some embodiments, the buffering agent is selected from HEPES, IVIES, Bis-tris methane, ADA, ACES, Bis-tris propane, PIPES, MOPSO, Cholamine chloride, MOPS, BES, TES, DIPSO, MOB, Acetamidoglycine, TAPSO, TEA, POPSO, HEPPSO, EPS, HEPPS, Tricine, Tris, Glycinamide, Glycylglycine, HEPBS, Bicine, TAPS, AMPB, CHES, AMP, AMPSO, CAPSO, CAPS and CABS. In some embodiments, the buffering agent in the formulation is at a concentration of from about 4 mM to about 400 mM, or from about 10 mM to about 300 mM, or from about 20 mM to about 200 mM, or from about 30 mM to about 100 mM, or from 35 mM to about 60 mM, or about 50 mM.

In some embodiments, the chelating agent is selected from EGTA, EDTA, DTPA, BAPTA, DMPS and ALA. In some embodiments, the chelating agent in the formulation is at a concentration of about 0.1 mM to about 10 mM, or from about 0.5 mM to about 5 mM, or about 1.25 mM.

In some embodiments, the nonionic surfactant is selected from Polyoxyethylene (20) sorbitan monolaurate (Tween-20), Polyoxyethylene (40) sorbitan monolaurate (Tween-40) and Polyoxyethylene (80) sorbitan monolaurate (Tween-80). In some embodiments, the nonionic surfactant is from about 0.1% to about 5% of the formulation, or about 0.2% to about 4% of the formulation, or from about 0.3% to about 3% of the formulation, or from about 0.4% to about 2% of the formulation, or about 0.5%, or about 1.5% of the formulation, or about 1.2% of the formulation, volume for volume.

In some embodiments, the pH of the formulation is from about pH 5 to about pH 9, or from about pH 6 to about pH 8, or from about pH 7 to about pH 7.9, or about pH 7.5.

In some embodiments, the formulation comprises 50 mM HEPES, 100 mM NaCl, 5 mM KCl, 8 mM MgCl2, 1.25 mM EGTA and 1.2% Tween-20.

In some embodiments, the formulation has a pH of about 7.5.

In some embodiments, the protease inhibitor is a reversible protease inhibitor. In some embodiments, the protease inhibitor inhibits proteases selected from trypsin, plasmin and thrombin. In some embodiments, the protease inhibitor is an inhibitor of serine protease. In some embodiments, the protease inhibitor is benzamidine. In some embodiments, the protease inhibitor in the formulation is at a concentration of about 0.1 mM to about 10 mM, or from about 0.5 mM to about 5 mM, or about 1.2 mM.

In some embodiments, the oligonucleotide is a single stranded oligonucleotide. In some embodiments, the oligonucleotide is from 20 to 100 nucleotides in length, or from 25 to 80 nucleotides in length, or from 25 to 70 nucleotides in length or from 25 to 50 nucleotides in length or about 30 nucleotides in length. In some embodiments, the oligonucleotide comprises one or more modified nucleotides. In some embodiments, the oligonucleotide comprises one or more C-5 modified pyrimidines. In some embodiments, the oligonucleotide comprises the sequence [(A-C-X-X)₇-A-C], wherein X is a BndU. In some embodiments, the oligonucleotide in the formulation has a concentration of from 5 μM to 100 μM or from 10 μM to 80 μM or from 20 μM to 60 μM or from 30 μM to 50 μM or about 75 μM or about 37 μM.

The sample volume to be dried may be determined by the device being used. By way of non-limiting example, the mitra VAMS microsampling device loads 30 μL so the sample starting volume is greater than 30 μL. In some embodiments, the sample volume loaded may be less that 30 μL. For example, 20 μL, 10 μL, or 5 μL. Sample volumes greater than 30 μL may also be used depending on the sample collection device.

As used herein “Catch-1” refers to the partitioning of an aptamer-target affinity complex or aptamer-target covalent complex. The purpose of Catch-1 is to remove substantially all of the components in the test sample that are not associated with the aptamer. Removing the majority of such components will generally improve target tagging efficiency by removing non-target molecules from the target tagging step used for Catch-2 capture and may lead to lower assay background. In one embodiment, a tag is attached to the aptamer either before the assay, during preparation of the assay, or during the assay by appending the tag to the aptamer. In one embodiment, the tag is a releasable tag. In one embodiment, the releasable tag comprises a cleavable linker and a tag. As described above, tagged aptamer can be captured on a solid support where the solid support comprises a capture element appropriate for the tag. The solid support can then be washed as described herein prior to equilibration with the test sample to remove any unwanted materials (Catch-0).

As used herein “Catch-2” refers to the partitioning of an aptamer-target affinity complex or aptamer-target covalent complex based on the capture of the target molecule. The purpose of the Catch-2 step is to remove free, or uncomplexed, aptamer from the test sample prior to detection and optional quantification. Removing free aptamer from the sample allows for the detection of the aptamer-target affinity or aptamer-target covalent complexes by any suitable nucleic acid detection technique. When using Q-PCR for detection and optional quantification, the removal of free aptamer is needed for accurate detection and quantification of the target molecule.

In one embodiment, the target molecule is a protein or peptide and free aptamer is partitioned from the aptamer-target affinity (or covalent) complex (and the rest of the test sample) using reagents that can be incorporated into proteins (and peptides) and complexes that include proteins (or peptides), such as, for example, an aptamer-target affinity (or covalent) complex. The tagged protein (or peptide) and aptamer-target affinity (or covalent) complex can be immobilized on a solid support, enabling partitioning of the protein (or peptide) and the aptamer-target affinity (or covalent) complex from free aptamer. Such tagging can include, for example, a biotin moiety that can be incorporated into the protein or peptide.

In one embodiment, a Catch-2 tag is attached to the protein (or peptide) either before the assay, during preparation of the assay, or during the assay by chemically attaching the tag to the targets. In one embodiment the Catch-2 tag is a releasable tag. In one embodiment, the releasable tag comprises a cleavable linker and a tag. It is generally not necessary, however, to release the protein (or peptide) from the Catch-2 solid support. As described above, tagged targets can be captured on a second solid support where the solid support comprises a capture element appropriate for the target tag. The solid support is then washed with various buffered solutions including buffered solutions comprising organic solvents and buffered solutions comprising salts and/or detergents containing salts and/or detergents.

After washing the second solid support, the aptamer-target affinity complexes are then subject to a dissociation step in which the complexes are disrupted to yield free aptamer while the target molecules generally remain bound to the solid support through the binding interaction of the capture element and target capture tag. The aptamer can be released from the aptamer-target affinity complex by any method that disrupts the structure of either the aptamer or the target. This may be achieved though washing of the support bound aptamer-target affinity complexes in high salt buffer which dissociates the non-covalently bound aptamer-target complexes. Eluted free aptamers are collected and detected. In another embodiment, high or low pH is used to disrupt the aptamer-target affinity complexes. In another embodiment high temperature is used to dissociate aptamer-target affinity complexes. In another embodiment, a combination of any of the above methods may be used. In another embodiment, proteolytic digestion of the protein moiety of the aptamer-target affinity complex is used to release the aptamer component.

In the case of aptamer-target covalent complexes, release of the aptamer for subsequent quantification is accomplished using a cleavable linker in the aptamer construct. In another embodiment, a cleavable linker in the target tag will result in the release of the aptamer-target covalent complex.

By way of example, the proteomic affinity assay (multiplex assay) may be practiced as follows:

Catch-0: 133 7.5% streptavidin-agarose slurry in l×SB17,Tw (40 mM HEPES, 102 mM NaCl, 1 mM EDTA, 5 mM MgCl2, 5 mM KCl, 0.05% Tween-20) was added to wells of the filter plate (0.45 μιη Millipore HV plates (Durapore cat #MAHVN4550)). The appropriate 1.1× aptamer mix (all aptamers contain a Cy3 fluorophore and a photocleavable biotin moiety on the 5′ end) was thawed followed by vortexing. The l.l× aptamer mix was then boiled for 10 min, vortexed for 30 s and allowed to cool to 20° C. in a water bath for 20 min. The liquid in the filter plates containing the streptavidin agarose slurry was then removed by centrifugation (1000×g for 1 minute). 100 μL aptamer mix was added to the wells of the filter plate (robotically). The mixture was incubated at 25° C. for 20 min on a shaker set at 850 rpm, protected from light.

Catch-0 washes: Subsequent to the 20 min incubation the solution was removed via vacuum filtration. 190 l×CAPS aptamer prewash buffer (50 mM CAPS, 1 mM EDTA, 0.05% Tw-20, pH 11.0) was added and the mixture was incubated for 1 minutes while shaking. The CAPS wash solution was then removed via vacuum filtration. The CAPS wash was then repeated one time. 190 μL l×SX17-Tween was added and the mixture was incubated for 1 min while shaking. The l×SB17-Tween was then removed via vacuum filtration. An additional 190 μL l×SX17-Tw was added and the mixture was incubated for 1 min while shaking. The l×SB17-Tw was then removed by centrifugation (1 min at 1000×g). Following removal of the l×SB17,Tw, 150 μL. Catch-0 storage buffer (150 mM NaCl, 40 mM HEPES, 1 mM EDTA, 0.02% sodium azide, 0.05% Tween-20) was added and the filter plate was carefully sealed at the plate perimeter only and stored at 4° C. in the dark until use.

Sample Preparation: Seventy-five (75) microliters of 40% sample diluent were plated out in a 40% sample plate (Final 40% sample contains: 20 M Z-block, 1 mM benzamidine, 1 mM EGTA, 40 mM HEPES, 5 mM MgCl2, 5 mM KCl, 1% Tween-20). One hundred ninety-five (195) microliters of l×SB17-Tw were plated out in a 1% sample plate. Ninety (90) microliters of l×SB17-Tw were plated out in a 1 to 10 dilution plate. One hundred thirty-three (133) microliters l×SB17-Tw were plated out in a 0.005% sample plate. Samples were thawed for 10 min on the Rack Thawing Station in a 25° C. incubator, then vortexed and spun at 1000×g for 1 minute. The caps were removed from the tubes. The samples were mixed (5 times with 50 L) and 50 μL 100% sample was transferred to the 40% sample plate containing the sample diluents. The 40% sample was then mixed on the sample plate by pipetting up and down (110 μL, 10 times). Five (5) L of 40% sample was then transferred to the 1% sample plate containing l×SB17-Tw. Again this sample was mixed by pipetting up and down (120 μL, 10 times). After mixing, 10 μL of the 1% sample was transferred to the 1 to 10 dilution plate containing l×SB17-Tw, which was mixed by pipetting up and down (75 μL, 10 times). Seven (7) microliters of the 0.1% sample from the 1 to 10 dilution plate was transferred into the 0.005% sample plate containing l×SB17-Tw and mixed by pipetting up and down (110 μL, 10 times).

Plate Preparation before Incubation: The Catch-0 storage solution was removed from the filter plates via vacuum filtration. One hundred ninety (190) microliters of l×SB17-Tw was then added followed by removal from the filter plates via vacuum filtration. An additional 190 μL l×SB17-Tw was then added to the filter plates.

Incubation: The l×SB17-Tw buffer was removed from the filter plates by centrifugation (1 min. at 1000×g). One hundred (100) microliters of the appropriate sample dilution was added to the filter plates (three filter plates, one for each sample dilution 40% or 20%, 1%, or 0.005%). The filter plates were carefully sealed at the plate perimeter only, avoiding pressurizing the wells. Pressure will cause leakage during incubation. The plates were then incubated for 3.5 hours at 28° C. on the thermoshaker set at 850 rpm, protected from light.

Filter Plate Processing: After incubation, the filter plates were placed onto vacuum manifolds and the sample was removed by vacuum filtration. One hundred ninety (190) microliters, biotin wash (100 M biotin in l×SB17-Tw) was added and the liquid was removed by vacuum filtration. The sample was then washed 5× with 190 μL l×SB17-Tw (vacuum filtration). One hundred (100) microliters of 1 mM NHS-biotin in l×SB17-Tw (freshly prepared) was added and the filter plates were blotted on an absorbent pad and the mixture was incubated for 5 minutes with shaking. The liquid was removed by vacuum filtration. One hundred and twenty five (125) microliters 20 mM glycine in l×SB17-Tw was added and the liquid was removed by vacuum filtration. Again 125 μL 20 mM glycine in l×SB17-Tw was added and the liquid removed by vacuum filtration.

Subsequently the samples were washed 6× with 190 μL l×SB17-Tw, with the liquid being removed by vacuum filtration. Eighty five (85) microliters of photocleavage buffer (2 M Z-block in l×SB17-Tw) was then added to each of the filter plates.

Photocleavage: The filter plates were blotted on absorbent pads and were irradiated for 6 min with a BlackRay UV lamp with shaking (800 rpm, 25° C.). The plates were rotated 180 degrees and irradiated for an additional 6 min. under the BlackRay light source. The 40% filter plate was placed onto an empty 96-well plate. The 1% filter plate was stacked on top of the 40% filter plate and the 0.005% filter plate was stacked on top of the 1% filter plate. The assembly of plated were spun for 1 min at 1000×g. The 96-well plate with eluted sample was placed onto the robot deck. Sixty (60) percent glycerol in l×SB17-Tw from the 37° C. incubator was placed onto the robotic deck.

Catch-2: During assay setup 50 μL of 10 mg/mL MyOne SA beads (500 g) was added to an ABgene Omni-tube 96-well plate for Catch-2 and placed in the Cytomat. The Catch-2 96-well bead plate was suspended for 90 s., placed on magnet block for 60 s. and the supernatant was removed. At the same time, or sequentially, the Catch-1 eluate from each dilution group was transferred to the Catch-2 bead plate and incubated on a Peltier thermoshaker (1350 rpm, 5 min, 25° C.). The plate was transferred to a 25° C. magnet for 2 minutes and the supernatant was removed. Next 75 μL l×SB17-Tw was added and the sample and incubated on a Peltier shaker at 1350 rpm for 1 minute at 37° C. Then 75 μL 60% glycerol in l×SB17-Tw (heated to 37° C.) was added and the sample was again incubated on the Peltier Shaker at 1350 rpm for 1 minute at 37° C. The plate was transferred to a magnet heated to 37° C. and incubated for 2 min. followed by the removal of the supernatant. This 37° C. l×SB17-Tw and glycerol wash cycle was repeated two more times. The sample was then washed to remove residual glycerol with 150 μL l×SB17-Tw on a Peltier shaker (1350 rpm, 1 minute, 25° C.), followed by 1 minute on a 25° C. magnetic block. The supernatant was removed and 150 μL l×SB17-Tw substituted with 0.5 M NaCl was added and incubated at 1350 rpm for 1 minute (25° C.) followed by 1 minute on a 25° C. magnetic block. The supernatant was removed and 75 μL perchlorate elution buffer (1.8 M NaClC-4, 40 mM PIPES, 1 mM EDTA, 0.05% Triton X-100, l×Hybridization controls, pH=6.8) was added followed by a 10 minute incubation on a Peltier shaker (25° C., 1350 rpm). Afterwards the plate was transferred to a magnetic separator and incubated for 90 s, and the supernatant was recovered.

Hybridization: Twenty (20) microliters eluted sample was added robotically to an empty the 96-well plate. Five (5) microliters 10× Agilent blocking buffer containing a second set of hybridization controls were robotically added to the eluted samples. Then 25 μL 2× Agilent HiRPM hybridization buffer was added manually to the wells. Forty (40) microliters of hybridization mix was loaded onto the Agilent gasket slide. The Agilent 8 by 15 k array was added onto gasket slide and the sandwich was tightened with a clamp. The sandwich was then incubated rotating (20 rpm) for 19 hours at 55° C.

Post-Hybridization Washing: Post hybridization slide processing was performed on a Little Dipper Processor (SciGene, Cat #1080-40-1). Approximately 750 mL wash buffer 1 (Oligo aCGH/ChlP-on-chip Wash Buffer 1, Agilent Technologies) was placed into one glass staining dish. Approximately 750 mL wash buffer 1 (Oligo aCGH/ChlP-on-chip Wash Buffer 1, Agilent Technologies) was placed into Bath #1 of the Little Dipper Processor. Approximately 750 mL wash buffer 2 (Oligo aCGH/ChlP-on-chip Wash Buffer 1, Agilent Technologies) heated to 37° C. was placed into Bath #2 of the Little Dipper Processor. The magnetic stir speed for both bath were set to 5. The temperature controller for Bath #1 was not turned on, while the temperature controller for Bath #2 was set to 37° C. Up to twelve slide/gasket assemblies were sequentially disassembled into the first staining dish containing Wash Buffer 1 and the slides were placed into a slide rack while still submerged in Wash Buffer 1. Once all slide/gaskets assemblies were disassembled, the slide rack was quickly transferred into Bath #1 of the Little Dipper Processor and the automated wash protocol was started. The Little Dipper Processor incubated the slides for 300 s. in Bath #1 at a speed of 250 followed by a transfer to the 37° C. Bath #2 containing the Agilent Wash 2 (Oligo aCGH/ChlP-on-chip Wash Buffer 2, Agilent Technologies) and incubated for 300 s. at speed 100. Afterwards the Little Dipper Processor transferred the slide rack to the built-in centrifuge, where the slides were spun for 300 s at speed 690.

Microarray Imaging: The microarray slides were imaged with a microarray scanner (Agilent G2565CA Microarray Scanner System, Agilent Technologies) in the Cy3-channel at 5 μιη resolution at 100% PMT setting and the XRD option enabled at 0.05. The resulting tiff images were processed using Agilent feature extraction software version 10.7.3.1 with the GEl_107_Sep09 protocol.

As used herein, a “releasable” or “cleavable” element, moiety, or linker refers to a molecular structure that can be broken to produce two separate components. A releasable (or cleavable) element may comprise a single molecule in which a chemical bond can be broken (referred to herein as an “inline cleavable linker”), or it may comprise two or more molecules in which a non-covalent interaction can be broken or disrupted (referred to herein as a “hybridization linker”).

In some embodiments, it is necessary to spatially separate certain functional groups from others in order to prevent interference with the individual functionalities. For example, the presence of a label, which absorbs certain wavelengths of light, proximate to a photocleavable group can interfere with the efficiency of photocleavage. It is therefore desirable to separate such groups with a non-interfering moiety that provides sufficient spatial separation to recover full activity of photocleavage, for example. In some embodiments, a “spacing linker” has been introduced into an aptamer with both a label and photocleavage functionality.

“Solid support” refers to any substrate having a surface to which molecules may be attached, directly or indirectly, through either covalent or non-covalent bonds. The solid support may include any substrate material that is capable of providing physical support for the capture elements or probes that are attached to the surface. The material is generally capable of enduring conditions related to the attachment of the capture elements or probes to the surface and any subsequent treatment, handling, or processing encountered during the performance of an assay. The materials may be naturally occurring, synthetic, or a modification of a naturally occurring material. Suitable solid support materials may include silicon, a silicon wafer chip, graphite, mirrored surfaces, laminates, membranes, ceramics, plastics (including polymers such as, e.g., poly(vinyl chloride), cyclo-olefin copolymers, agarose gels or beads, polyacrylamide, polyacrylate, polyethylene, polypropylene, poly(4-methylbutene), polystyrene, polymethacrylate, poly(ethylene terephthalate), polytetrafluoroethylene (PTFE or Teflon®), nylon, poly(vinyl butyrate)), germanium, gallium arsenide, gold, silver, Langmuir Blodgett films, a flow through chip, etc., either used by themselves or in conjunction with other materials. Additional rigid materials may be considered, such as glass, which includes silica and further includes, for example, glass that is available as Bioglass. Other materials that may be employed include porous materials, such as, for example, controlled pore glass beads, crosslinked beaded Sepharose® or agarose resins, or copolymers of crosslinked bis-acrylamide and azalactone. Other beads include nanoparticles, polymer beads, solid core beads, paramagnetic beads, or microbeads. Any other materials known in the art that are capable of having one or more functional groups, such as any of an amino, carboxyl, thiol, or hydroxyl functional group, for example, incorporated on its surface, are also contemplated.

The material used for a solid support may take any of a variety of configurations ranging from simple to complex. The solid support can have any one of a number of shapes, including a strip, plate, disk, rod, particle, bead, tube, well (microtiter), and the like. The solid support may be porous or non-porous, magnetic, paramagnetic, or non-magnetic, polydisperse or monodisperse, hydrophilic or hydrophobic. The solid support may also be in the form of a gel or slurry of closely-packed (as in a column matrix) or loosely-packed particles.

In one embodiment, the solid support with attached capture element is used to capture tagged aptamer-target affinity complexes or aptamer-target covalent complexes from a test mixture. In one particular example, when the tag is a biotin moiety, the solid support could be a streptavidin-coated bead or resin such as Dynabeads M-280 Streptavidin, Dynabeads MyOne Streptavidin, Dynabeads M-270 Streptavidin (Invitrogen), Streptavidin Agarose Resin (Pierce), Streptavidin Ultralink Resin, MagnaBind Streptavidin Beads (ThermoFisher Scientific), BioMag Streptavidin, ProMag Streptavidin, Silica Streptavidin (Bangs Laboratories), Streptavidin Sepharose High Performance (GE Healthcare),

Streptavidin Polystyrene Microspheres (Microspheres-Nanospheres), Streptavidin Coated Polystyrene Particles (Spherotech), or any other streptavidin coated bead or resin commonly used by one skilled in the art to capture biotin-tagged molecules.

As has been described above, one object of the instant invention is to convert a protein signal into an aptamer signal. As a result the quantity of aptamers collected/detected is indicative of, and may be directly proportional to, the quantity of target molecules bound and to the quantity of target molecules in the sample. A number of detection schemes can be employed without eluting the aptamer-target affinity or aptamer-target covalent complex from the second solid support after Catch-2 partitioning. In addition to the following embodiments of detection methods, other detection methods will be known to one skilled in the art.

Many detection methods require an explicit label to be incorporated into the aptamer prior to detection. In these embodiments, labels, such as, for example, fluorescent or chemiluminescent dyes can be incorporated into aptamers either during or post synthesis using standard techniques for nucleic acid synthesis. Radioactive labels can be incorporated either during synthesis or post synthesis using standard enzyme reactions with the appropriate reagents. Labeling can also occur after the Catch-2 partitioning and elution by using suitable enzymatic techniques. For example, using a primer with the above mentioned labels, PCR will incorporate labels into the amplification product of the eluted aptamers. When using a gel technique for quantification, different size mass labels can be incorporated using PCR as well. These mass labels can also incorporate different fluorescent or chemiluminescent dyes for additional multiplexing capacity. Labels may be added indirectly to aptamers by using a specific tag incorporated into the aptamer, either during synthesis or post synthetically, and then adding a probe that associates with the tag and carries the label. The labels include those described above as well as enzymes used in standard assays for colorimetric readouts, for example. These enzymes work in combination with enzyme substrates and include enzymes such as, for example, horseradish peroxidase (HRP) and alkaline phosphatase (AP). Labels may also include materials or compounds that are electrochemical functional groups for electrochemical detection.

For example, the aptamer may be labeled, as described above, with a radioactive isotope such as 32 P prior to contacting the test sample. Employing any one of the four basic assays, and variations thereof as discussed above, aptamer detection may be simply accomplished by quantifying the radioactivity on the second solid support at the end of the assay. The counts of radioactivity will be directly proportional to the amount of target in the original test sample. Similarly, labeling an aptamer with a fluorescent dye, as described above, before contacting the test sample allows for a simple fluorescent readout directly on the second solid support. A chemiluminescent label or a quantum dot can be similarly employed for direct readout from the second solid support, requiring no aptamer elution.

By eluting the aptamer or releasing photoaptamer-target covalent complex from the second solid support additional detection schemes can be employed in addition to those described above. For example, the released aptamer, photoaptamer or photoaptamer-target covalent complex can be run on a PAGE gel and detected and optionally quantified with a nucleic acid stain, such as SYBR Gold. Alternatively, the released aptamer, photoaptamer or photoaptamer covalent complex can be detected and quantified using capillary gel electrophoresis (CGE) using a fluorescent label incorporated in the aptamer as described above. Another detection scheme employs quantitative PCR to detect and quantify the eluted aptamer using SYBR Green, for example. Alternatively, the Invader® DNA assay may be employed to detect and quantify the eluted aptamer. Another alternative detection scheme employs next generation sequencing.

In another embodiment, the amount or concentration of the aptamer-target affinity complex (or aptamer-target covalent complex) is determined using a “molecular beacon” during a replicative process (see, e.g., Tyagi et ah, Nat. Biotech. J_6.49 53, 1998; U.S. Pat. No. 5,925,517). A molecular beacon is a specific nucleic acid probe that folds into a hairpin loop and contains a fluorophore on one end and a quencher on the other end of the hairpin structure such that little or no signal is generated by the fluorophore when the hairpin is formed. The loop sequence is specific for a target polynucleotide sequence and, upon hybridizing to the aptamer sequence the hairpin unfolds and thereby generates a fluorescent signal.

For multiplexed detection of a small number of aptamers still bound to the second solid support, fluorescent dyes with different excitation/emission spectra can be employed to detect and quantify two, or three, or five, or up to ten individual aptamers.

Similarly different sized quantum dots can be employed for multiplexed readouts. The quantum dots can be introduced after partitioning free aptamer from the second solid support. By using aptamer specific hybridization sequences attached to unique quantum dots multiplexed readings for 2, 3, 5, and up to 10 aptamers can be performed. Labeling different aptamers with different radioactive isotopes that can be individually detected, such as 32 P, 3 H, 113JC, and 3 J5JS, can also be used for limited multiplex readouts.

For multiplexed detection of aptamers released from the Catch-2 second solid support, a single fluorescent dye, incorporated into each aptamer as described above, can be used with a quantification method that allows for the identification of the aptamer sequence along with quantification of the aptamer level. Methods include but are not limited to DNA chip hybridization, micro-bead hybridization, next generation sequencing and CGE analysis.

In one embodiment, a standard DNA hybridization array, or chip, is used to hybridize each aptamer or photoaptamer to a unique or series of unique probes immobilized on a slide or chip such as Agilent arrays, Illumina BeadChip Arrays, NimbleGen arrays or custom printed arrays. Each unique probe is complementary to a sequence on the aptamer. The complementary sequence may be a unique hybridization tag incorporated in the aptamer, or a portion of the aptamer sequence, or the entire aptamer sequence. The aptamers released from the Catch-2 solid support are added to an appropriate hybridization buffer and processed using standard hybridization methods. For example, the aptamer solution is incubated for 12 hours with a DNA hybridization array at about 60° C. to ensure stringency of hybridization. The arrays are washed and then scanned in a fluorescent slide scanner, producing an image of the aptamer hybridization intensity on each feature of the array. Image segmentation and quantification is accomplished using image processing software, such as ArrayVision. In one embodiment, multiplexed aptamer assays can be detected using up to 25 aptamers, up to 50 aptamers, up to 100 aptamers, up to 200 aptamers, up to 500 aptamers, up to 1000 aptamers, and up to 10,000 aptamers.

In one embodiment, addressable micro-beads having unique DNA probes complementary to the aptamers as described above are used for hybridization. The micro-beads may be addressable with unique fluorescent dyes, such as Luminex beads technology, or use bar code labels as in the Illumina VeraCode technology, or laser powered transponders. In one embodiment, the aptamers released from the Catch-2 solid support are added to an appropriate hybridization buffer and processed using standard micro-bead hybridization methods. For example, the aptamer solution is incubated for two hours with a set of micro-beads at about 60° C. to ensure stringency of hybridization. The solutions are then processed on a Luminex instrument which counts the individual bead types and quantifies the aptamer fluorescent signal. In another embodiment, the VeraCode beads are contacted with the aptamer solution and hybridized for two hours at about 60° C. and then deposited on a gridded surface and scanned using a slide scanner for identification and fluorescence quantification. In another embodiment, the transponder micro-beads are incubated with the aptamer sample at about 60° C. and then quantified using an appropriate device for the transponder micro-beads. In one embodiment, multiplex aptamer assays can be detected by hybridization to micro-beads using up to 25 aptamers, up to 50 aptamers, up to 100 aptamers, up to 200 aptamers, and up to 500 aptamers.

The sample containing the eluted aptamers can be processed to incorporate unique mass tags along with fluorescent labels as described above. The mass labeled aptamers are then injected into a CGE instrument, essentially a DNA sequencer, and the aptamers are identified by their unique masses and quantified using fluorescence from the dye incorporated during the labeling reaction. One exemplary example of this technique has been developed by Althea Technologies.

In many of the methods described above, the solution of aptamers can be amplified and optionally tagged before quantification. Standard PCR amplification can be used with the solution of aptamers eluted from the Catch-2 solid support. Such amplification can be used prior to DNA array hybridization, micro-bead hybridization, and CGE readout.

In another embodiment, the aptamer-target affinity complex (or aptamer-target covalent complex) is detected and/or quantified using Q-PCR. As used herein, “Q-PCR” refers to a PCR reaction performed in such a way and under such controlled conditions that the results of the assay are quantitative, that is, the assay is capable of quantifying the amount or concentration of aptamer present in the test sample.

In one embodiment, the amount or concentration of the aptamer-target affinity complex (or aptamer-target covalent complex) in the test sample is determined using TaqMan® PCR. This technique generally relies on the 5′-3′ exonuclease activity of the oligonucleotide replicating enzyme to generate a signal from a targeted sequence. A TaqMan probe is selected based upon the sequence of the aptamer to be quantified and generally includes a 5′-end fluorophore, such as 6-carboxyfluorescein, for example, and a 3′-end quencher, such as, for example, a 6-carboxytetramethylfluorescein, to generate signal as the aptamer sequence is amplified using polymerase chain reaction (PCR). As the polymerase copies the aptamer sequence, the exonuclease activity frees the fluorophore from the probe, which is annealed downstream from the PCR primers, thereby generating signal. The signal increases as replicative product is produced. The amount of PCR product depends upon both the number of replicative cycles performed as well as the starting concentration of the aptamer.

In another embodiment, the amount or concentration of an aptamer-target affinity complex (or aptamer-target covalent complex) is determined using an intercalating fluorescent dye during the replicative process. The intercalating dye, such as, for example, SYBR® green, generates a large fluorescent signal in the presence of double-stranded DNA as compared to the fluorescent signal generated in the presence of single-stranded DNA. As the double-stranded DNA product is formed during PCR, the signal produced by the dye increases. The magnitude of the signal produced is dependent upon both the number of PCR cycles and the starting concentration of the aptamer.

In another embodiment, the aptamer-target affinity complex (or aptamer-target covalent complex) is detected and/or quantified using mass spectrometry. Unique mass tags can be introduced using enzymatic techniques described above. For mass spectroscopy readout, no detection label is required, rather the mass itself is used to both identify and, using techniques commonly used by those skilled in the art, quantified based on the location and area under the mass peaks generated during the mass spectroscopy analysis. An example using mass spectroscopy is the MassARRAY® system developed by Sequenom.

A computer program may be utilized to carry out one or more steps of any of the methods disclosed herein. Another aspect of the present disclosure is a computer program product comprising a computer readable storage medium having a computer program stored thereon which, when loaded into a computer, performs or assists in the performance of any of the methods disclosed herein.

One aspect of the disclosure is a product of any of the methods disclosed herein, namely, an assay result, which may be evaluated at the site of the testing or it may be shipped to another site for evaluation and communication to an interested party at a remote location, if desired. As used herein, “remote location” refers to a location that is physically different than that at which the results are obtained. Accordingly, the results may be sent to a different room, a different building, a different part of city, a different city, and so forth. The data may be transmitted by any suitable means such as, e.g., facsimile, mail, overnight delivery, e-mail, ftp, voice mail, and the like.

“Communicating” information refers to the transmission of the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

### Modified Nucleotides

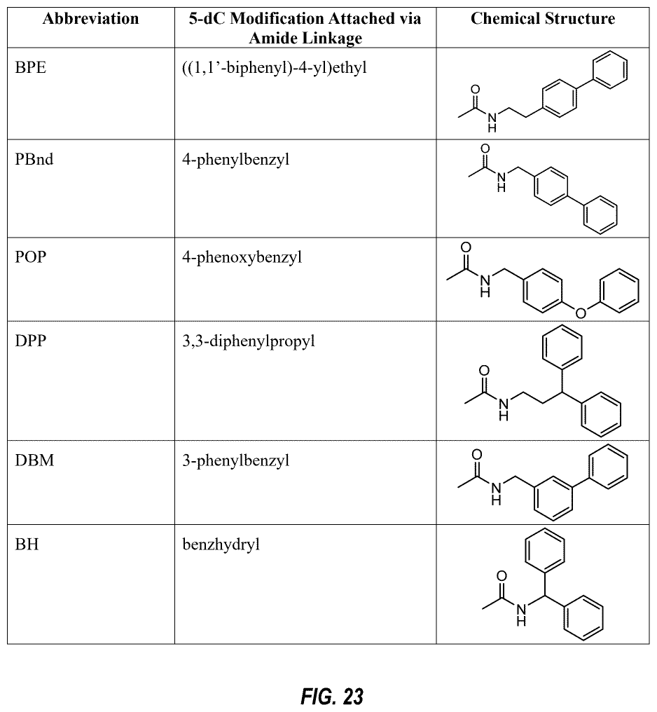

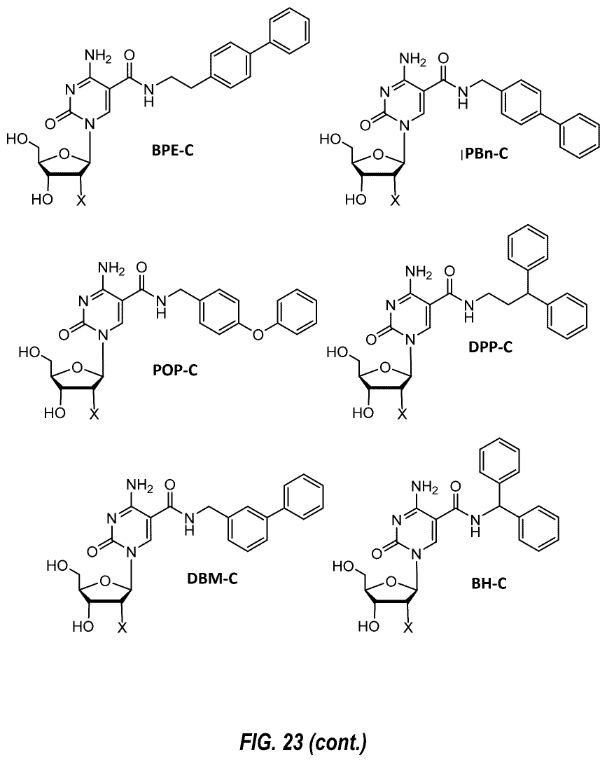

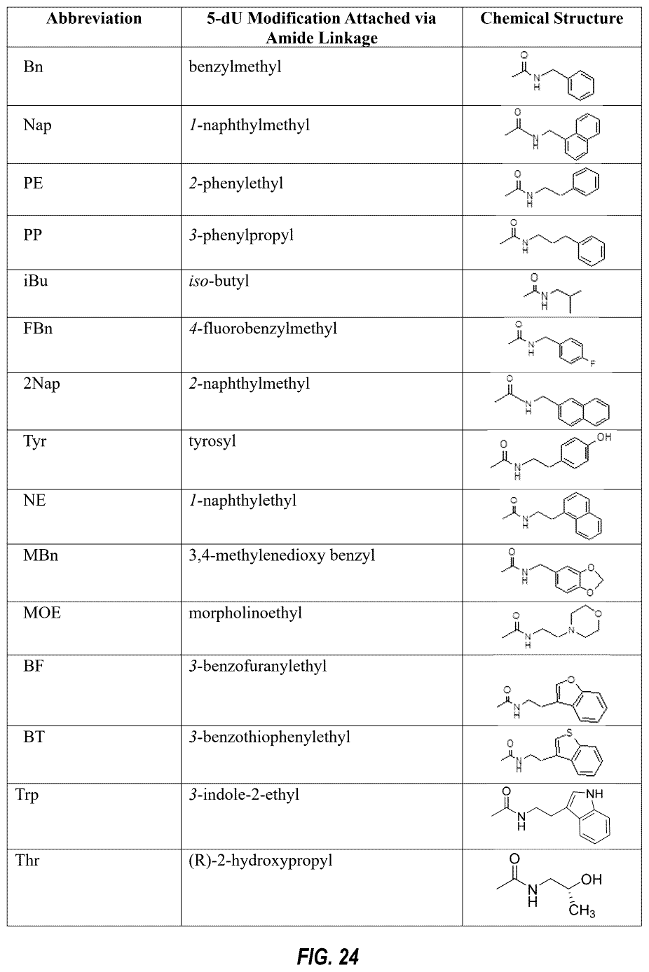

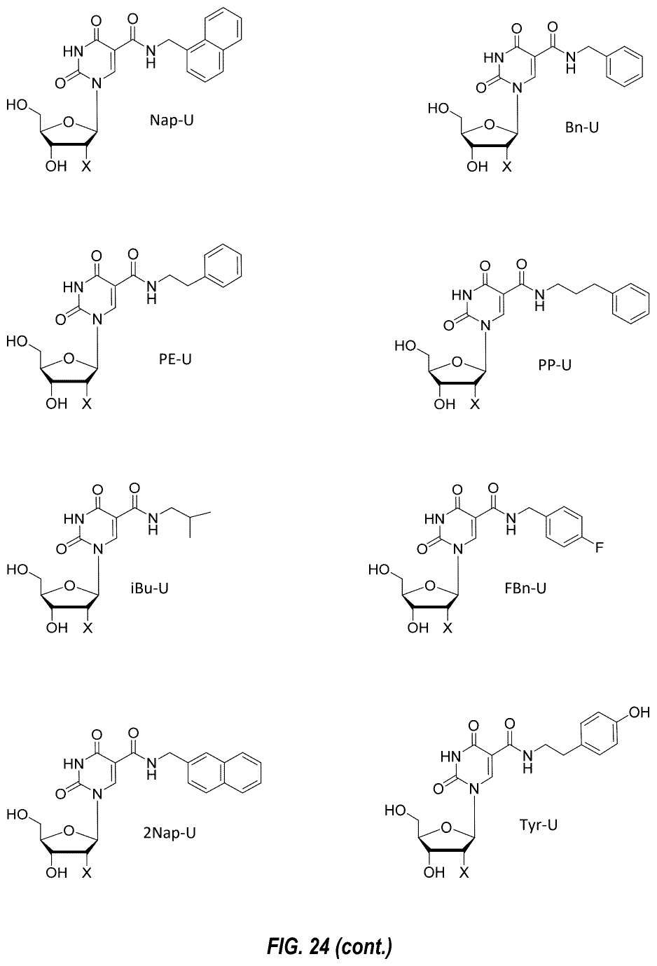

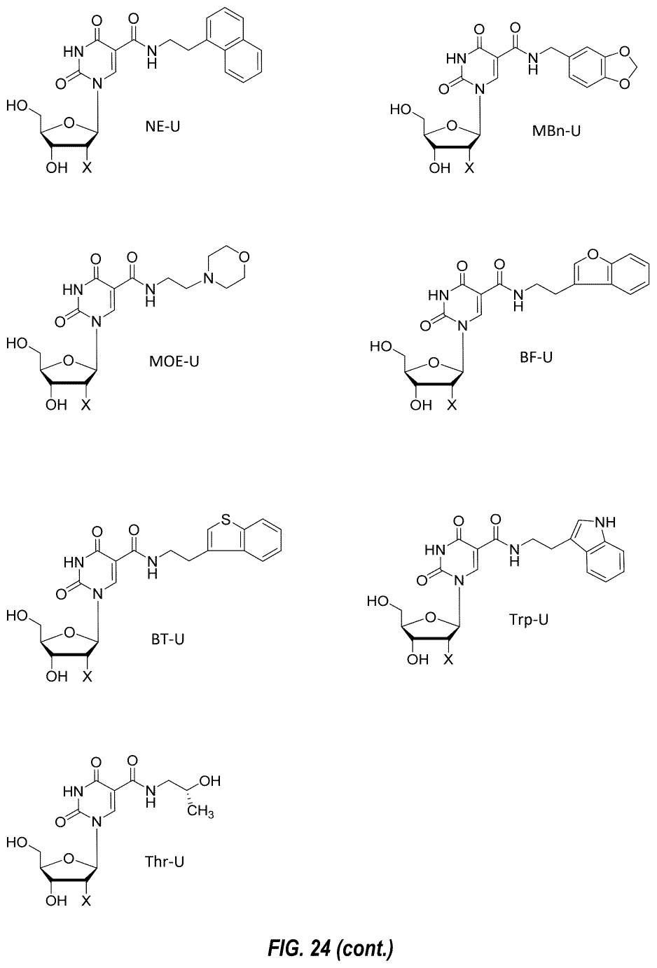

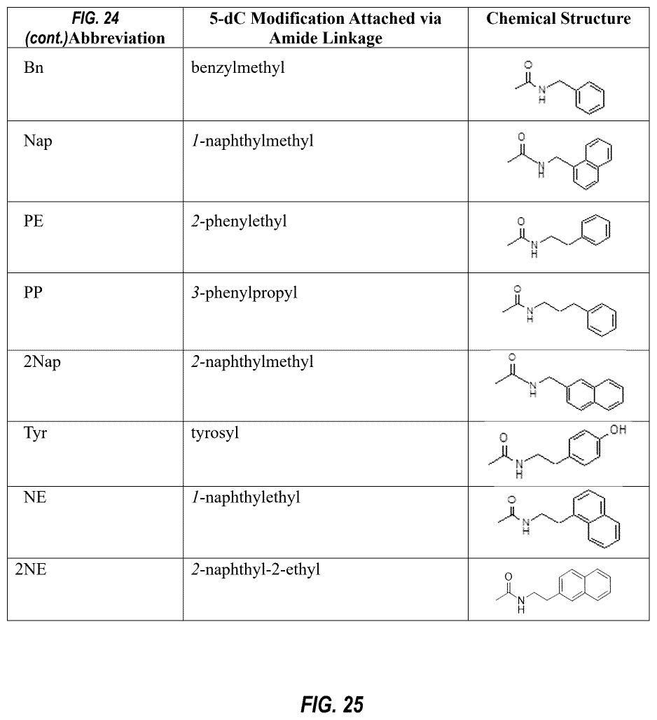

In certain embodiments, the disclosure provides oligonucleotides, such as aptamers, which comprise two different types of base-modified nucleotides. In some embodiments, the oligonucleotides comprise two different types of 5-position modified pyrimidines. In some embodiments, the oligonucleotide comprises at least one C5-modified cytidine and at least one C5-modified uridine. In some embodiments, the oligonucleotide comprises two different C5-modified cytidines. In some embodiments, the oligonucleotide comprises two different C5-modified uridines. Nonlimiting exemplary C5-modified uridines and cytidines are shown, for example, in FIG. 21. Certain nonlimiting exemplary C5-modified uridines are shown in FIGS. 22 and 24, and certain non-limiting exemplary C5-modified cytidines are shown in FIGS. 23 and 25.

### Preparation of Oligonucleotides

The automated synthesis of oligodeoxynucleosides is routine practice in many laboratories (see e.g., Matteucci, M. D. and Caruthers, M. H., (1990) J. Am. Chem. Soc., 103:3185-3191, the contents of which are hereby incorporated by reference in their entirety). Synthesis of oligoribonucleosides is also well known (see e.g. Scaringe, S. A., et al., (1990) Nucleic Acids Res. 18:5433-5441, the contents of which are hereby incorporated by reference in their entirety). As noted herein, the phosphoramidites are useful for incorporation of the modified nucleoside into an oligonucleotide by chemical synthesis, and the triphosphates are useful for incorporation of the modified nucleoside into an oligonucleotide by enzymatic synthesis. (See e.g., Vaught, J. D. et al. (2004) J. Am. Chem. Soc., 126:11231-11237; Vaught, J. V., et al. (2010) J. Am. Chem. Soc. 132, 4141-4151; Gait, M. J. “Oligonucleotide Synthesis a practical approach” (1984) IRL Press (Oxford, UK); Herdewijn, P. “Oligonucleotide Synthesis” (2005) (Humana Press, Totowa, N.J. (each of which is incorporated herein by reference in its entirety).

“Target” or “target molecule” or “target” refers herein to any compound upon which a nucleic acid can act in a desired or intended manner. A target molecule can be a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc., without limitation. Virtually any chemical or biological effector may be a suitable target. Molecules of any size can serve as targets. A target can also be modified in certain ways to enhance the likelihood or strength of an interaction between the target and the nucleic acid. A target can also include any minor variation of a particular compound or molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules. Embodiments of the SELEX process in which the target is a peptide are described in U.S. Pat. No. 6,376,190, entitled “Modified SELEX Processes Without Purified Protein.” In some embodiments, a target is a protein.

As used herein, “competitor molecule” and “competitor” are used interchangeably to refer to any molecule that can form a non-specific complex with a non-target molecule. In this context, non-target molecules include free aptamers, where, for example, a competitor can be used to inhibit the aptamer from binding (rebinding), non-specifically, to another non-target molecule. A “competitor molecule” or “competitor” is a set of copies of one type or species of molecule. “Competitor molecules” or “competitors” refer to more than one such set of molecules. Competitor molecules include, but are not limited to oligonucleotides, polyanions (e.g., heparin, herring sperm DNA, salmon sperm DNA, tRNA, dextran sulfate, polydextran, abasic phosphodiester polymers, dNTPs, and pyrophosphate). In various embodiments, a combination of one or more competitor can be used.

As used herein, “non-specific complex” refers to a non-covalent association between two or more molecules other than an aptamer and its target molecule. A non-specific complex represents an interaction between classes of molecules. Non-specific complexes include complexes formed between an aptamer and a non-target molecule, a competitor and a non-target molecule, a competitor and a target molecule, and a target molecule and a non-target molecule.

In another embodiment, a polyanionic competitor (e.g., dextran sulfate or another polyanionic material) is used in the slow off-rate enrichment process to facilitate the identification of an aptamer that is refractory to the presence of the polyanion. In this context, “polyanionic refractory aptamer” is an aptamer that is capable of forming an aptamer/target complex that is less likely to dissociate in the solution that also contains the polyanionic refractory material than an aptamer/target complex that includes a nonpolyanionic refractory aptamer. In this manner, polyanionic refractory aptamers can be used in the performance of analytical methods to detect the presence or amount or concentration of a target in a sample, where the detection method includes the use of the polyanionic material (e.g. dextran sulfate) to which the aptamer is refractory.

Thus, in one embodiment, a method for producing a polyanionic refractory aptamer is provided. In this embodiment, after contacting a candidate mixture of nucleic acids with the target. The target and the nucleic acids in the candidate mixture are allowed to come to equilibrium. A polyanionic competitor is introduced and allowed to incubate in the solution for a period of time sufficient to insure that most of the fast off rate aptamers in the candidate mixture dissociate from the target molecule. Also, aptamers in the candidate mixture that may dissociate in the presence of the polyanionic competitor will be released from the target molecule. The mixture is partitioned to isolate the high affinity, slow off-rate aptamers that have remained in association with the target molecule and to remove any uncomplexed materials from the solution. The aptamer can then be released from the target molecule and isolated. The isolated aptamer can also be amplified and additional rounds of selection applied to increase the overall performance of the selected aptamers. This process may also be used with a minimal incubation time if the selection of slow off-rate aptamers is not needed for a specific application.

### Salts

It may be convenient or desirable to prepare, purify, and/or handle a corresponding salt of the compound, for example, a pharmaceutically-acceptable salt. Examples of pharmaceutically acceptable salts are discussed in Berge et al. (1977) “Pharmaceutically Acceptable Salts” J. Pharm. Sci. 66:1-19.

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R^{X+}, NH₂R^{X}₂⁺, NHR^{X}₃⁺, NR^{X}₄⁺). Examples of some suitable substituted ammonium ions are those derived from: ethylamine, diethylamine, dicyclohexylamine, triethylamine, butylamine, ethylenediamine, ethanolamine, diethanolamine, piperizine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Other Embodiments

In some embodiments, a method is disclosed comprising a) contacting a first test sample with a first set of aptamers to form a first mixture, wherein the first test sample is a Z % dilution of the biological sample, wherein Z is from 0.1% to 10%. dilution of a biological sample, and there are at least A₃ different aptamers in the first set of aptamers; b) contacting a second test sample with a second set of aptamers to form a second mixture, wherein the second test sample is a Y % dilution of the biological sample, wherein Y is less than Z, wherein Y is from 0.001% to 0.1%, and wherein there are at least A₂ different aptamers in the second set of aptamers; c) contacting a third test sample with a third set of aptamers to form a third mixture, wherein the third test sample is a X % dilution of the biological sample, wherein X is 0.001% to 0.1%, and there are at least A₁ different aptamers in the third set of aptamers; d) incubating the first, second and third mixtures to allow for the formation of aptamer-protein complexes, and removing a majority of the aptamers that did not form aptamer-protein complexes; e) collecting the aptamers from the aptamer-protein complexes by dissociating the aptamer-protein complexes; f) detecting or quantifying the collected aptamers; wherein, a majority of the aptamers of the first set of aptamers, second set of aptamers and third set of aptamers each have affinity for a different target protein in the test sample, and are capable of forming a aptamer-protein complex with its target protein, and wherein A₃ is greater than A₂, and A₂ is greater than A₂; and wherein the sum of A₁, A₂ and A₃ is at least 4,000.

In one aspect, sum of A₁, A₂ and A₃ is at least 4,500 or 5,000.

In one aspect, A₃ is from 50% to 90% (or 50%, 55%, 60%, 65%, 70%, 75%, 80%, 85% or 90%) of the sum of A₁, A₂ and A₃; or from 60% to 85% of the sum of A₁, A₂ and A₃; or about 80% or 81% of the sum of A₁, A₂ and A₃.

In one aspect, A₂ is from 10% to 49% (or 10%, 15%, 20%, 25%, 30%, 35%, 40%, 45% or 49%) of the sum of A₁, A₂ and A₃; or from 12% to 35% of the sum of A₁, A₂ and A₃; or from 15% to 30% of the sum of A₁, A₂ and A₃; or about 15% or 16% of the sum of A₁, A₂ and A₃.

In one aspect, A₁ is from 1% to 9% (or 1%, 2%, 3%, 4%, 5%, 6%, 7%, 8% or 9%) of the sum of A₁, A₂ and A₃; or from 2% to 7% of the sum of A₁, A₂ and A₃; or from 3% to 6% of the sum of A₁, A₂ and A₃; or about 3% or 4% of the sum of A₁, A₂ and A₃.

In one aspect, A₃ is at least 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4200, 4270, 4500, 5000 (or is from 900 to 16,500 or from 2000 to 15,000 or from 3,000 to 12,000 or from 4,000 to 10,000).

In one aspect, A₂ is at least 200, 250, 300, 350, 400, 450, 500, 550, 600, 650, 700, 750, 800, 820, 900 (or is from 500 to 3500 or from 700 to 2500, or from 800 to 2000).

In one aspect, A₁ is at least 100, 110, 120, 130, 140, 150, 160, 170, 173 (or is from 100 to 700 or 100 to 650).

In one aspect, the at least A₃ different aptamers are differ from one another by at least one nucleotide differences and/or at least one nucleotide modification.

In one aspect, the at least A₂ different aptamers are differ from one another by at least one nucleotide differences and/or at least one nucleotide modification.

In one aspect, the at least A₁ different aptamers are differ from one another by at least one nucleotide differences and/or at least one nucleotide modification.

In one aspect, the at least A₃ different aptamers, the at least A₂ different aptamers and the at least A₁ different aptamers are differ from one another by at least one nucleotide differences and/or at least one nucleotide modification.

In some embodiments, a system is disclosed comprising a) a first receptacle having a first mixture comprising a first test sample with a first set of aptamers, wherein the first test sample is an Z % dilution of a test sample, and there are at least A₃ different aptamers in the first set of aptamers; b) a second receptacle having a second mixture comprising a second test sample with a second set of aptamers, wherein the second test sample is a Y % dilution of the test sample, wherein Y is less than or equal to Z, and there are at least A₂ different aptamers in the second set of aptamers; c) a third receptacle having a third mixture comprising a third test sample with a third set of aptamers, wherein the third test sample is a X % dilution of the test sample, wherein X is less than or equal to Y, and there are at least A₁ different aptamers in the third set of aptamers; and wherein, a majority of the aptamers of the first set of aptamers, second set of aptamers and third set of aptamers have affinity for a protein in the test sample, and are capable of forming a aptamer-protein complex, and wherein A₃ is greater than A₂, and A₂ is greater than A₁; and wherein the sum of A₁, A₂ and A₃ is at least 4,000; and wherein, the system is used to detect proteins in the test sample, and the first, second and third test samples are a different dilution of the same test sample.

In one aspect, the plurality of first capture reagents is about 100, 110, 120, 130, 140, 150, 160, 170 or 173; or is from 100 to 700; or from 100 to 650 capture reagents.

In one aspect, the plurality of second capture reagents is about 200, 250, 300, 350, 400, 450, 500, 550, 600, 650, 700, 750, 800, 820 or 900; or is from 500 to 3500; or is from about 700 to 2500; or is from 800 to 2000; or about 828 capture reagents.

In one aspect, the plurality of third capture reagents is about 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4200, 4270, 4500 or 5000; or is from about 900 to 16,500; or from about 2000 to 15,000; or from about 3,000 to 12,000; or from about 4,000 to 10,000; or about 4271 capture reagents.

### EXAMPLES

The following examples are presented in order to more fully illustrate some embodiments of the disclosure. They should, in no way be construed, however, as limiting the broad scope of the disclosure. Those of ordinary skill in the art can readily adopt the underlying principles of this discovery to design various compounds without departing from the spirit of the current disclosure.

### Example 1. Multiplexed Aptamer Assay Analysis of Samples

A multiplex aptamer assay was used to analyze test samples and control samples to examine the detection of target molecules from dried biologic matrices. The multiplexed analysis used in this experiment included aptamers to detect approximately 5,000 proteins eluted from dried biologic matrix samples, with low limits of detection (1 pM median), ~7 logs of dynamic range, and ~5% median coefficient of variation. The multiplex aptamer assay is described, generally, e.g., in Gold et al. (2010) Aptamer-Based Multiplexed Proteomic Technology for Biomarker Discovery. PLoS ONE 5(12): e15004; U.S. Publication Nos: 2012/0101002 and 2012/0077695; and PCT Publication No. WO 2019/246289.

### Example 2: Optimization of Multiplex Assay Dilutions with Dried Blood Spot (DBS) Extracts

This example provides a description of optimization of dilutions of dried blood spot extracts in a multiplex assay that maximizes the number of analytes in the linear range while still maintaining the greatest median signal to background signal ratio in a multiplex assay.

In a multiplex assay format where multiple target proteins are being measured by multiple capture reagents, the natural variation in the abundance of the different target proteins can limit the ability of certain capture reagents to measure certain target proteins (e.g., high abundance target proteins may saturate the assay and prevent or reduce the ability of the assay to measure low abundance target proteins). To address this variation in the biological sample, the aptamer reagents are separated into at least two different groups, preferably three different groups, based on the abundance of their respective protein target in the biological sample. The biological sample is diluted into at least two, preferably three, different dilution groups to create separate test samples based on relative concentrations of the protein targets to be detected by their capture reagents. Thus, the biological sample may be diluted into high and low abundant target protein dilution groups, or high, medium and low abundant target protein dilution groups, where the least abundant protein targets are measured in the least diluted group, and the most abundant protein targets are measured in the greatest diluted group. In this example, the aptamers were grouped into three unique mixes, Dil1, Dil2 and Dil3.

Methods: Venous blood was collected into 2, 4 mL BD Vacutainer tubes containing K₂EDTA from three healthy volunteers. One tube from each donor was spun at 2,200×g for 15 minutes and approximately 2 mL of platelet poor plasma was generated. The second tube was not spun but instead used for the collection and drying of venous whole blood.

Whole blood (100 μL) was spotted onto parafilm paper and the tip of the Mitra VAMS™ microsampling device was placed into the whole blood sample until the sample collection device was fully saturated with an additional 2 seconds of dwell time. This was performed four times, one for each tip included in the sample collection device clam packaging. The same procedure was repeated for plasma. Mitra packaging devices were closed and placed into a sealed container with eight DRIERITE desiccants for three days at room temperature.

Dried tips from each clam were placed into 3 mL tubes (4 per tube) with 600 μL elution buffer (80% plasma diluent and 20% PBS). Plasma diluent is 50 mM Hepes, pH 7.5, 100 mM NaCl, 8 mM MgCl2, 5 mM KCl, 1.25 mM EGTA, 1.2 mM Benzamidine, 37.5 μM Z-Block and 1.2% Tween-20. The samples were allowed to elute at room temperature for 1 h with shaking at 1000 rpm. After 1 h, the buffer was gently pipetted up and down to mix before being diluted. Extracts from each donor (450 μL) were pooled into a master stock of WB and a master stock of plasma.

In order to determine the proper dilution for dilution group 1, serial dilutions (1:2) in plasma diluent were performed by transferring 400 μL of whole blood extract in the first 1 mL well of a 96 well plate and then transferring 200 μL into successive wells containing 200 μL of 80% Plasma Diluent and 20% PBS. This was repeated fourteen times. These served as the dilutions for dilution group 1. Dilutions were made in the multiplex assay for dilution groups 2 (40× dilution from dilution 1) and 3 (100× dilution from dilution 2).

| | | | Volume | Number | |
|---|---|---|---|---|---|
| | | | of Diluent | of | |
| Device | Matrix | Donor | (uL) | Samples | Dilution |
| Mitra | Plasma | 9003 | 600 | 4 | 5x |
| Mitra | Whole Blood | 9003 | 600 | 4 | 5x |
| Mitra | Plasma | 9004 | 600 | 4 | 5x |
| Mitra | Whole Blood | 9004 | 600 | 4 | 5x |
| Mitra | Plasma | 9005 | 600 | 4 | 5x |
| Mitra | Whole Blood | 9005 | 600 | 4 | 5x |

In order to determine the proper dilutions for dilution group 2 and dilution group 3, serial dilutions were prepared in 1 mL volume, 96 well plates. Serial dilutions (1:2) in assay buffer were performed by starting with 800 μL of 20% extract in plasma diluent and PBS and transferring 400 μL of extract and mixing it with 400 μL of assay buffer. The multiplex assay was performed by directly pipetting 100 μL of each dilution directly into each catch plate.

The dilutions were separated into two submissions for the multiplex assay.

Submission I

| | 1 | | 2 | |
|---|---|---|---|---|
| A | 20 | 0.0781 | 20 | 0.0781 |
| B | 10 | 0.0391 | 10 | 0.0391 |
| C | 5 | 0.0195 | 5 | 0.0195 |
| D | 2.5 | 0.010 | 2.5 | 0.010 |
| E | 1.25 | 0.005 | 1.25 | 0.005 |
| F | 0.625 | 0.002 | 0.625 | 0.002 |
| G | 0.3125 | 0.001 | 0.3125 | 0.001 |
| H | 0.156 | 0.000 | 0.156 | 0.000 |

| | |
|---|---|
| WB, Plasma | Plasma, Plasma |
| Diluent Dilution | Diluent Dilution |

Submission II

| | 1 | | 2 | |
|---|---|---|---|---|
| A | 20 | 0.0781 | 20 | 0.0781 |
| B | 10 | 0.0391 | 10 | 0.0391 |
| C | 5 | 0.0195 | 5 | 0.0195 |
| D | 2.5 | 0.010 | 2.5 | 0.010 |
| E | 1.25 | 0.005 | 1.25 | 0.005 |
| F | 0.625 | 0.002 | 0.625 | 0.002 |
| G | 0.3125 | 0.001 | 0.3125 | 0.001 |
| H | 0.156 | 0.000 | 0.156 | 0.000 |

| | |
|---|---|
| WB, Assay Buffer | Plasma, Assay |
| Dilution | Buffer Dilution |

### Results

The optimal concentration to run an assay is within the linear range. This avoids problems of saturation, measuring background noise, or unknown factors that could confound the results of an assay. Therefore, it is important to choose a dilution for a matrix that places as many of its analytes within the linear range as possible.

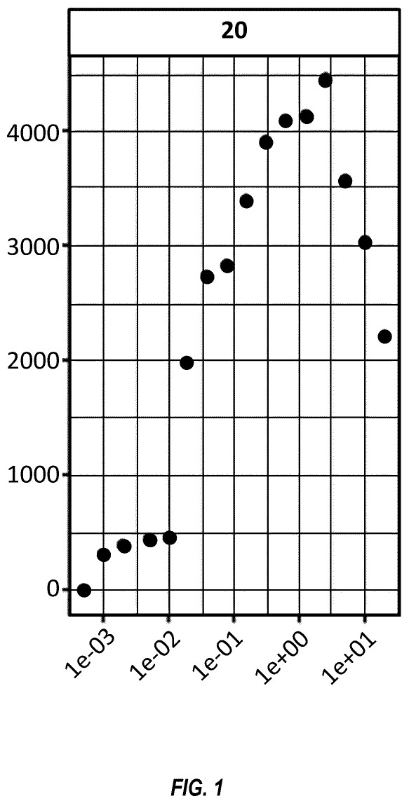

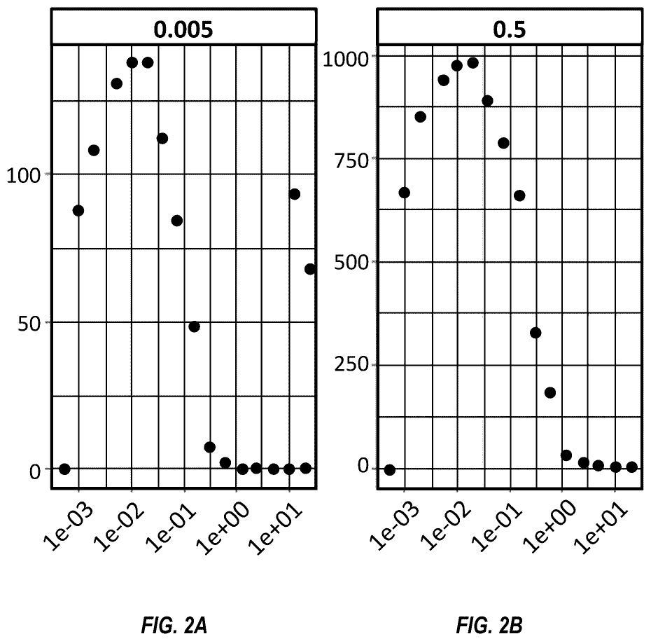

The linearity algorithm looks at each analyte over the course of all 15 dilutions and determines whether doubling the concentration of plasma doubles the signal (plus or minus 25%). An analyte must contain at least three dilutions where the signal is linear before being counted by the algorithm. At each dilution, the number of signals within the linear range is tallied, generating FIGS. 1 and 2A and 2B. The optimal concentration (the dilution where the most analytes experience a linear signal response) was determined by diluting dried blood spot (DBS) extract in plasma diluent and PBS (80/20). Based on the plot shown in FIG. 1, the optimal concentration range for dilution group 1 exists between 0.3% and 5%. The optimal concentration (the dilution where the most analytes experience a linear signal response) of dilution groups 2 and 3 were determined by taking DBS extracted in plasma diluent and PBS (80/20) and diluting into assay buffer. Based on the plots shown in FIGS. 2A and 2B, the optimal concentration range for dilution group 2 exists between 0.005% and 0.038% and the optimal concentration range for dilution group 3 exists between 0.005% and 0.038%.

The maximum allowable dilution of a sample is marked by the loss of too many analyte signals as they drop below the level of background noise in the multiplex assay. Background in the multiplex assay can be defined as a multiple of the standard deviation of repeated measurements of a signal when only buffer is analyzed.

Every analyte in the multiplex assay gives a background signal in buffer and every assay is run with at least three buffer samples in order to determine the standard deviation of that background signal. For any given dilution of DBS or plasma, a signal is considered to be “above background” when it is 3.3 standard deviations above the mean background signal. In statistical terms analytes with signal intensities of this magnitude have only a 0.1% chance of being due to noise.

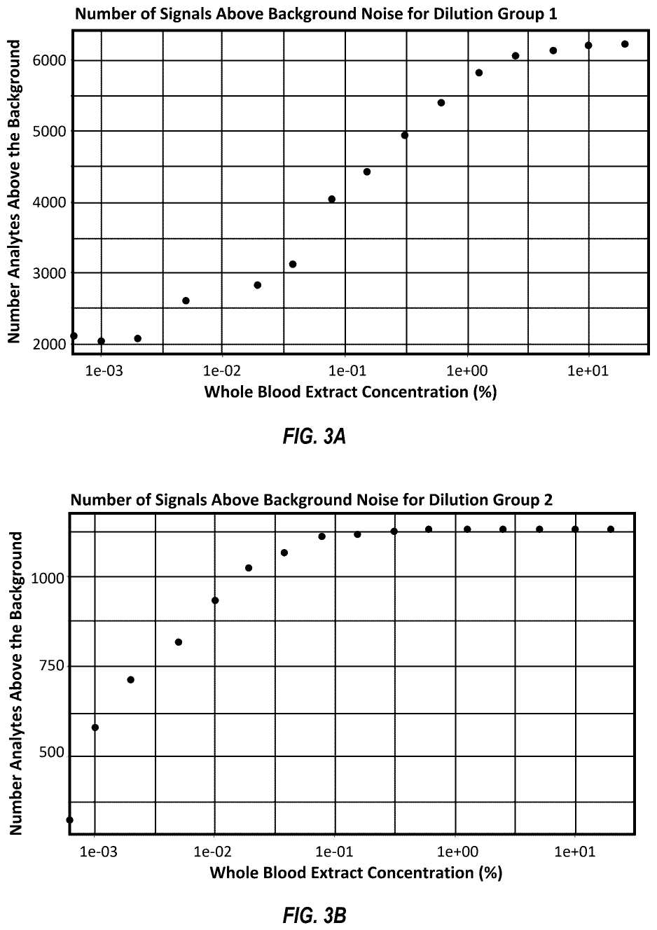

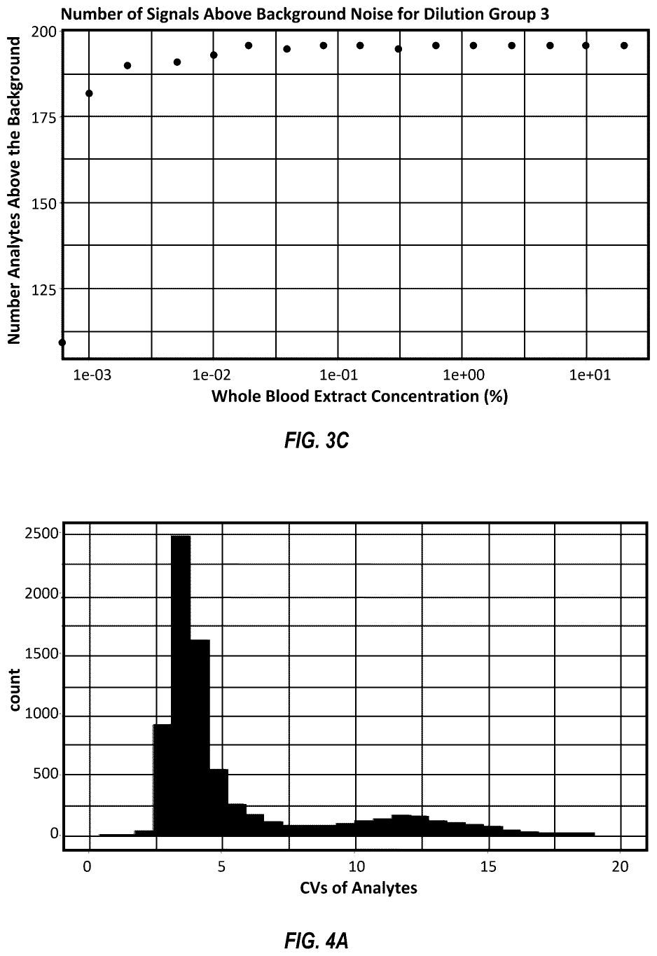

As shown in FIG. 3A, almost no signals are lost to noise in dilution group 1 at a 20% or 10% concentration of DBS extract. Dilution Group 1 does not lose a significant number of analytes to the background in DBS until after the extracts are diluted below 1.25%. As shown in FIG. 3B, almost no signals are lost to noise in dilution group 2 until the concentration of extract sinks below 0.3% concentration of DBS extract. Dilution Group 2 does not lose a significant number of analytes to the background in extracts until after the extracts are diluted below 0.038%. As shown in FIG. 3C, virtually no signals are lost to noise in dilution group 3 until the concentration of extract sinks below 0.018% concentration of whole blood extract. Dilution Group 3 does not lose a significant number of analytes to the background in whole blood until after the extracts are diluted below 0.005%.

### Example 3: Precision of Dried Blood Spots

Materials: Whatman 903™, Guthrie Cards. Plasma Diluent. PBS. BD Vacutainer Tubes (K₂EDTA).

Methods: A small population of six donors were drawn with K₂EDTA vacutainer tubes and their blood divided into five assay replicates to obtain an intra-plate assay CV. Plasma was also collected from these donors and three replicates were analyzed.

### Blood Collection/Preparation/Handling:

Six donors were drawn into one of three K₂EDTA, BD vacutainer tubes (whole blood, plasma, and spare). Plasma tubes from each donor were spun at 2,200×g for 15 minutes and approximately 2 mL of plasma (PPP) was removed and stored in two 1.5 mL Eppendorf tubes and immediately frozen on dry ice.

### Loading Guthrie Cards and Drying:

Three Guthrie cards (Whatman 903™, LOT 7211021) were spotted for each donor. Using a micropipette, 40 μL of whole blood was spotted onto the center of the dotted circles of three different Guthrie cards (five spots per card, three cards per donor). Cards were allowed to dry in a laminar flow hood for 1 hour (folding the card to ensure the back side of the spotted paper was not in contact with any surface) before being placed into 6 different sealed bags (three cards per bag) containing two desiccants per bag. Cards were allowed to dry for three days at room temperature.

### Elution:

After three days of drying one Guthrie card was removed from each bag. The remainder were stored at −20° C. as a backup. The dried blood spots were excised from their cards with scissors, being sure to remove 100% of the dried blood spot. Five circles of whole blood were placed in five 2 mL screw cap tubes. 1.6 mL of extraction buffer (80% Plasma Dil and 20% PBS) was added to each tube and they were shaken for 1 h at 1000 rpm at room temperature. This was done for each donor for a total of thirty tubes.

Dilution group 1 samples were transferred (200 μL from each extract) into 30 matrix tubes and frozen at −80° C. An additional 600 μL were transferred into 30 different matrix tubes for use with dilution groups 2 and 3.

One plasma sample from each donor was thawed and 50 μL was transferred into three matrix tubes for each donor (sample replicates). An additional 200 μL of extraction buffer was added to dilute to 20%. Three plasma QCs, eight blanks (5 serial numbers not recorded), and 5 calibrators were diluted to 20% in extraction buffer in the same way. Dilution 1 samples were thawed for the multiplex assay. All extracts and plasma samples were stored on ice.

Linearity of multiplex assay signals increases with optimized dilutions for whole blood that are less concentrated than the optimal dilutions for plasma or serum. Dried whole blood samples from all six donors was prepared with dilutions of 2.5%, 0.05%, and 0.005%. Liquid plasma from all six donors was prepared with dilutions of 20%, 0.5%, and 0.005%. Samples were refrozen and submitted for analysis by the multiplex assay.

| | |
|---|---|
| Dried Whole Blood | Liquid Plasma |

| | |
|---|---|
| Buffer | Concentrations |

| | | | |
|---|---|---|---|
| Dil 1 | Plasm Dil/ | 2.50% | 20% |
| | PBS | | |
| Dil 2 | Assay | 0.05% | 0.5% |
| | Buffer | | |
| Dil 3 | Assay | 0.005% | 0.005% |
| | Buffer | | |

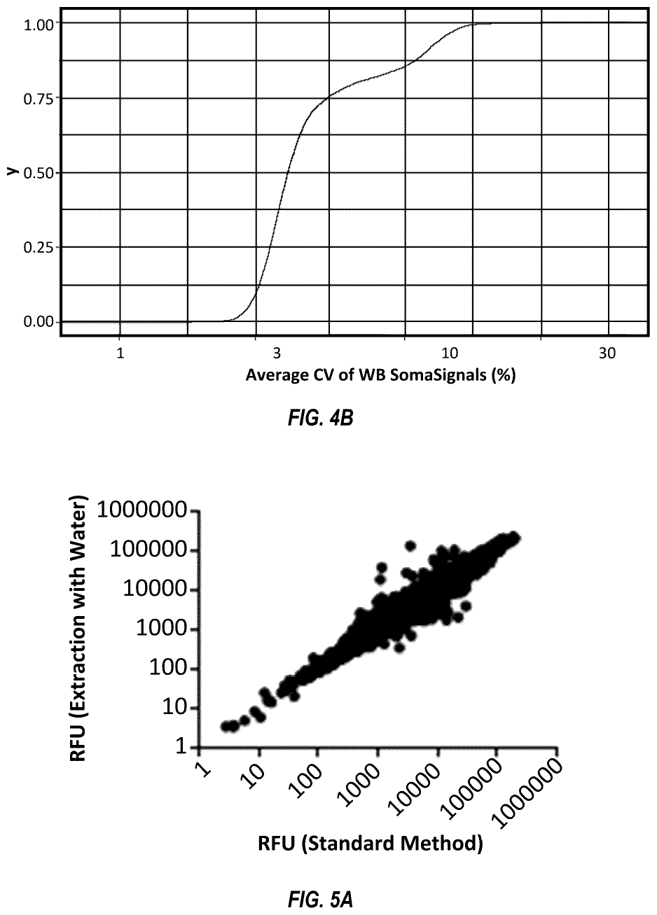

Results: The global average precision for all analytes tested is described in FIGS. 4A and 4B. Population Coefficient of Variation (CVs) of analytes displayed a bimodal distribution with the majority of analytes (>70%) having a coefficient of variation of less than 5%. A second population of analytes had a greater degree of imprecision with a broader distribution of CVs. Over 95% of analytes showed an imprecision of less than 15% between replicates as shown in FIG. 4A.

The imprecision of whole blood analytes is divided into two populations, high and medium precision. The high precision population represent the majority of analytes tested, that are tightly distributed around 3% CV with a distribution of 2.5%-7.5%. Medium precision analytes represent around 25% of the library with a range of 7.5%-17.5%, centered around 11%. The bulk of these analytes have CVs of less than 15%, which is promising for medical applications.

Both populations of CVs can be seen in the CDF plot shown in FIG. 4B. CDF plots are integrated histograms (the area under the curve) and convey the same information.

### Example 4: Robustness of Protein Signals in Whole Blood Determined by Protein Spikes

Methods: A large library of proteins were spiked into DBS extract in order to determine whether or not increases in protein concentration can be measured across analytes in this matrix.

### Blood Collection/Preparation/Handling:

Venous blood was collected into 2, 6 mL BD Vacutainer tubes containing K₂EDTA from donor 1301, stored at room temperature, and processed within two hours. One tube was spun at 2,200×g for 15 minutes to create a layer of platelet poor plasma.

### Loading Mitra Device:

100 μL of whole blood and plasma (respectively) was spotted onto parafilm paper and the tip of the Mitra VAMS collection device was placed into it until the sample collection device was fully saturated plus an additional 2 seconds. This was performed 16 times for whole blood (four clams) and 4 times for plasma (1 clam). These devices were placed into sealed containers with two DRIERITE desiccants per clam for three days at room temperature.

Protein Doping into WB Extracts

After three days, whole blood treated Mitra tips were soaked in plasma extraction buffer (20% PBS, 80% plasma diluent, 300 μL per tip) for 1 h under shaking. The 10× (10%) extract was aliquoted into duplicates and spiked with the protein spike library (Appendix II). Plasma loaded tips were prepared the same way. Samples were prepared in 150 μL aliquots with a substitution of the spike library in: 10%, 5%, 1%, 0% substitutions.

Samples were frozen and run in the multiplex assay.

Results: Two non-spiked dried whole blood samples demonstrate high levels of concordance between dried whole blood extracts collected by the Mitra VAMs system when derived from the same donor. When a non-spiked whole blood sample is compared with one with a roughly 5 nM, or 5% substitution, spike from the protein library, the added proteins “lift” up from the line of identity. This trend is also visible from similarly spiked plasma samples.

Titration curves for the duplicate whole blood extracts and plasma extracts were measured with increasing percent protein substitution. With background signals subtracted, a sigmoidal dose response is revealed. Interestingly not all elements in the spike library appear to experience an increase in signal. This may be due to the fact that these proteins were more easily denatured or experienced a greater level of imprecision in whole blood. There may also be sensitivity issues for a subset of proteins within the spike library, leading to a lack of detection for certain members.

Median signal increase for spike library targets was measured and compared for whole blood and plasma. A median of 232 signal increases over baseline was obtained. The signal increases are almost identical between whole blood and plasma.

The signal increase for each of the 232 spike analytes between 0 nM and 5 nM for plasma was divided by the equivalent increase in whole blood. If plasma and whole blood respond the same way to a protein spike, then there should be a distribution of signals around 1. Both whole blood replicates show that on an analyte by analyte basis, the two matrices respond the same to proteins being spiked in their extract.

### Example 5: Extraction Optimization Studies

Dried samples must be reconstituted to achieve approximately the same buffer conditions as for a typical plasma or serum sample submitted for assay. In the typical multiplex assay scheme, one-part biological sample is added to 4 parts of the serum diluent 50 mM Hepes, pH 7.5, 100 mM NaCl, 8 mM MgCl2, 5 mM KCl, 1.25 mM EGTA, 1.2 mM Benzamidine, 75 μM Z-Block and 1.2% Tween-20) or plasma diluent (50 mM Hepes, pH 7.5, 100 mM NaCl, 8 mM MgCl2, 5 mM KCl, 1.25 mM EGTA, 1.2 mM Benzamidine, 37.5 μM Z-Block and 1.2% Tween-20) and 100 μL added to wells containing Mix 1 beads. Thus, for a 30 μL dried sample, extraction took place in 150 μL of a buffer solution containing 4 parts diluent and 1-part of a volume expander. The volume expanders tested were water and phosphate buffered saline (PBS). An experiment was conducted to compare these two-types of volume expanders. Here 12 serum samples (QC2) were thawed and Mitra VAMS samples collected and dried for 2.5 days at 4° C. Half of the tips were extracted for 1 hour at room temperature (RT) using water and the other half were extracted for 1 hour at room temperature using PBS. Extracts from duplicate tips were combined resulting in triplicate samples for each condition. Multiplex assay from the two extraction methods were compared to each other and each to a standard QC2 serum sample (not dried) that had undergone an additional freeze-thaw cycle to equalize the number of freeze-thaw cycles of all samples.

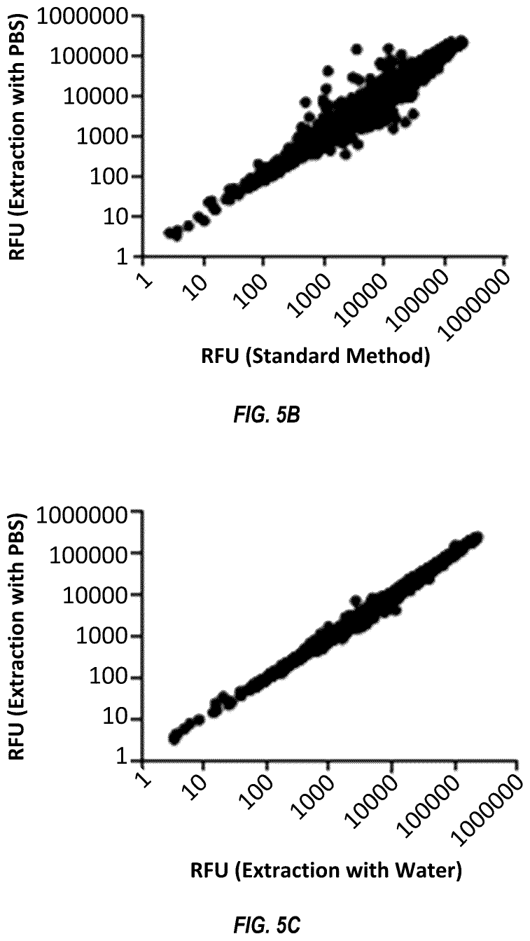

Concordance plots of the relative fluorescent units (RFU) between the two extraction conditions show little difference (FIG. 5). Likewise, cumulative distribution plots of the median RFU of the standard method and the two extraction conditions from the dried spots are nearly superimposable with a slight benefit in the symmetry of the fit when using the PBS method (not shown). Furthermore, the cumulative distribution plot of the percent coefficient of variation (% CV) also reveals a slight benefit when using the PBS method. Both methods of extracting the dried spots showed increased % CV relative to the standard method. PBS and water are suitable volume expanders for extraction of dried samples.

### Example 6: Determination of Protein Extraction Time

Methods: Ten QC1 and ten QC2 serum samples (v4.0) were thawed and Mitra samples collected and dried for 2.5 days at 4° C. Samples were extracted using PBS and serum diluent as the volume expander as described above. Duplicate Mitra tips from each QC sample were removed and placed in a microtiter well and extracted with rotation at RT for 10, 30, 60, 120, or 180 minutes. Extracts from duplicate tips (at each timepoint) were combined and frozen until tested in the multiplex assay along with standard QC1 and QC2 serum samples (not dried) that had undergone an additional freeze-thaw cycle to equalize the number of freeze-thaw cycles of all samples. As a blank control, Mitra VAMS samples of the extraction buffer were collected and dried in an identical manner and tested in the multiplex assay along with standard multiple assay blanks.

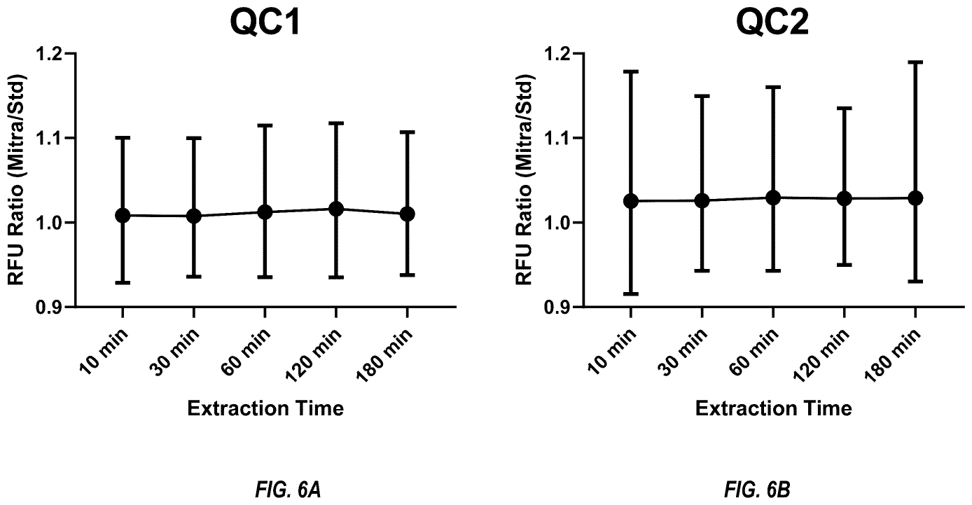

Results: Recovery of analyte signals were largely independent of extraction time over the range of 10 to 180 minutes. Shown in FIG. 6 are the median (interquartile range) of the ratio of analyte signals from the dried spots (Mitra) to those obtained from the same sample analyzed by the multiplex assay method (not dried). The median ratio varied little over the range of extraction times and was slightly greater than 1 in all cases. Cumulative distribution plots of the RFU signals obtained for dried serum samples extracted for the indicated length of time at room temperature show analyte signals obtained from the five extraction times are nearly superimposable with one another and with those obtained from a standard serum sample (not dried). Analyte signals obtained from the QC1 blank sample undergoing the drying and extraction process were superimposable with the signals obtained using a standard multiplex blank sample. An extraction time between about 10 minutes and 200 minutes is acceptable for the multiplex assay. In some embodiments, a 60 minute extraction time is used.

### Example 7: Demonstration of Precision and Consistent Concordance with Dried Serum

Methods: To gain a better understanding of the reproducibility of dried plasma spots, an experiment was conducted using fresh serum obtained from six normal volunteers in which all Mitra VAMS samples were dried under identical conditions. Blood was collected and allowed to clot for 1 hour at room temperature. Clotted samples were spun at 2,200×g for 15 minutes and an aliquot of serum transferred to an Eppendorf tube. From each serum sample, duplicate Mitra VAMS tips (30 μL) were collected and dried in the dark at RT for 4 days placed into sealed containers with DRIERITE desiccants. In addition, aliquots of the fresh serum were frozen until assayed for comparison to the dried spots. Extractions were performed for 1 hour with water as the volume expander and extracts from the duplicate Mitra VAMS tips were combined and frozen at −80° C. until assayed. Extracts and controls were assayed in a multiplex assay.

Results: Concordance plots between the standard method and the dried method for each of six individual serum samples show that all analytes still signal after drying and reconstitution. Although the concordance isn't perfect, what matters most is if the ratio of each analyte signal is constant across samples e.g. if an analyte always returns half or twice the signal as obtained by the standard method. Since these ratios are constant, i.e. there are acceptable CV's for all ratios, then the results from the dried spot can be mathematically converted, “lifted”, to match the expected results from the non-dried sample.

The % CV of the ratio for each analyte was determined and is plotted as a cumulative distribution function. The median % CV of the ratios was 6.99 with 10th and 90th percentiles of 3.59 and 18.8, respectively demonstrating that when dried and processed under identical conditions most analyte ratios do remain reasonably constant across samples.

### Example 8: The Stability of Dried Plasma to Temperature Changes Based on Drying Time and Temperature

### Methods:

Blood Collection/Preparation/Handling: Venous blood was collected into 2, 6 mL BD Vacutainer tubes containing K2EDTA from a healthy volunteer, stored at room temperature, and processed within the two hours. One tube was spun at 2,200×g for 15 minutes and platelet poor plasma was removed. Plasma (400 μL) was frozen at −80° C. for use as a control.

Loading Mitra Device: 80 μL of whole blood or plasma was spotted onto parafilm paper and the tip of the Mitra VAMS was placed into it until the sample collection device was fully saturated (~2 sec).

Drying Conditions: Plasma samples were placed in sealed bags (two drierite containers per Mitra clam) and placed in 3 different temperature conditions for drying: −20° C., 4° C., and 25° C. The Mitra VAMS devices were dried for one to three days before either being removed and extracted in 20% PBS and 80% plasma diluent or placed at a stressing temperature.

Stressing Temperatures: After drying from 1 to 3 days, samples were removed from their drying conditions and allowed to reach room temperature. Then they were placed in their stressing conditions at either −20° C., 37° C., 50° C., or 60° C. for either 1 or 2 days. After either 1 or 2 days the samples were removed, and the plasma was extracted in 20% PBS and 80% Plasma Diluent.

Extraction: Mitra tips were placed individually in Eppendorf tubes containing of 300 μL extraction buffer (80% plasma diluent and 20% PBS) and allowed to extract for 1 h under shaking. The contents were transferred to matrix tubes and frozen at −80° C.

| Drying | Drying | Stress | Stress | Drying | Drying | Stress | Stress | Drying | Drying | Stress | Stress |
|---|---|---|---|---|---|---|---|---|---|---|---|
| Time | Temp | Time | Temp | Time | Temp | Time | Temp | Time | Temp | Time | Temp |
| 1 DAY | −20° C. | No Stress | N/A | 2 DAY | −20° C. | No Stress | N/A | 3 DAY | −20° C. | No Stress | N/A |
| | | | N/A | | | | N/A | | | | N/A |
| | | | N/A | | | | N/A | | | | N/A |
| | | | N/A | | | | N/A | | | | N/A |
| | | 1 Day | −20° C. | | | 1 Day | −20° C. | | | 1 Day | −20° C. |
| | | | 37° C. | | | | 37° C. | | | | 37° C. |
| | | | 50° C. | | | | 50° C. | | | | 50° C. |
| | | | 60° C. | | | | 60° C. | | | | 60° C. |
| | | 2 Day | −20° C. | | | 2 Day | −20° C. | | | 2 Day | −20° C. |
| | | | 37° C. | | | | 37° C. | | | | 37° C. |
| | | | 50° C. | | | | 50° C. | | | | 50° C. |
| | | | 60° C. | | | | 60° C. | | | | 60° C. |
| | 4° C. | No Stress | N/A | | 4° C. | No Stress | N/A | | 4° C. | No Stress | N/A |
| | | | N/A | | | | N/A | | | | N/A |
| | | | N/A | | | | N/A | | | | N/A |
| | | | N/A | | | | N/A | | | | N/A |
| | | 1 Day | −20° C. | | | 1 Day | −20° C. | | | 1 Day | −20° C. |
| | | | 37° C. | | | | 37° C. | | | | 37° C. |
| | | | 50° C. | | | | 50° C. | | | | 50° C. |
| | | | 60° C. | | | | 60° C. | | | | 60° C. |
| | | 2 Day | −20° C. | | | 2 Day | −20° C. | | | 2 Day | −20° C. |
| | | | 37° C. | | | | 37° C. | | | | 37° C. |
| | | | 50° C. | | | | 50° C. | | | | 50° C. |
| | | | 60° C. | | | | 60° C. | | | | 60° C. |
| | 25° C. | No Stress | N/A | | 25° C. | No Stress | N/A | | 25° C. | No Stress | N/A |
| | | | N/A | | | | N/A | | | | N/A |
| | | | N/A | | | | N/A | | | | N/A |
| | | | N/A | | | | N/A | | | | N/A |
| | | 1 Day | −20° C. | | | 1 Day | −20° C. | | | 1 Day | −20° C. |
| | | | 37° C. | | | | 37° C. | | | | 37° C. |
| | | | 50° C. | | | | 50° C. | | | | 50° C. |
| | | | 60° C. | | | | 60° C. | | | | 60° C. |
| | | 2 Day | −20° C. | | | 2 Day | −20° C. | | | 2 Day | −20° C. |
| | | | 37° C. | | | | 37° C. | | | | 37° C. |
| | | | 50° C. | | | | 50° C. | | | | 50° C. |
| | | | 60° C. | | | | 60° C. | | | | 60° C. |

Results: Plasma-loaded Mitra VAMS devices were dried at different temperatures, stressed at different temperatures as shown above, and then compared via concordance plots to liquid plasma that had been stored at −80 TC. The best results are achieved when plasma is dried in the cold, however this could be simply due to the greater degree of temperature stability found in a refrigerator and freezer. The concordance between all dried samples is high but dried plasma extract align best with liquid plasma when the samples are dried at 4° C. or −20° C. as opposed to room temperature, although the concordance at room temperature is also acceptable for the multiplex assay.

Concordance of cardiovascular disease analytes (CVD2) between samples that have been dried at 4° C., −20° C., and room temp was measured. There are 28 analytes in the CVD2 test and this subset of proteins appears to have the greatest concordance to liquid plasma when dried at −20° C. The concordance to liquid plasma is also acceptable for samples dried at 4° C. and room temperature for the multiplex assay.

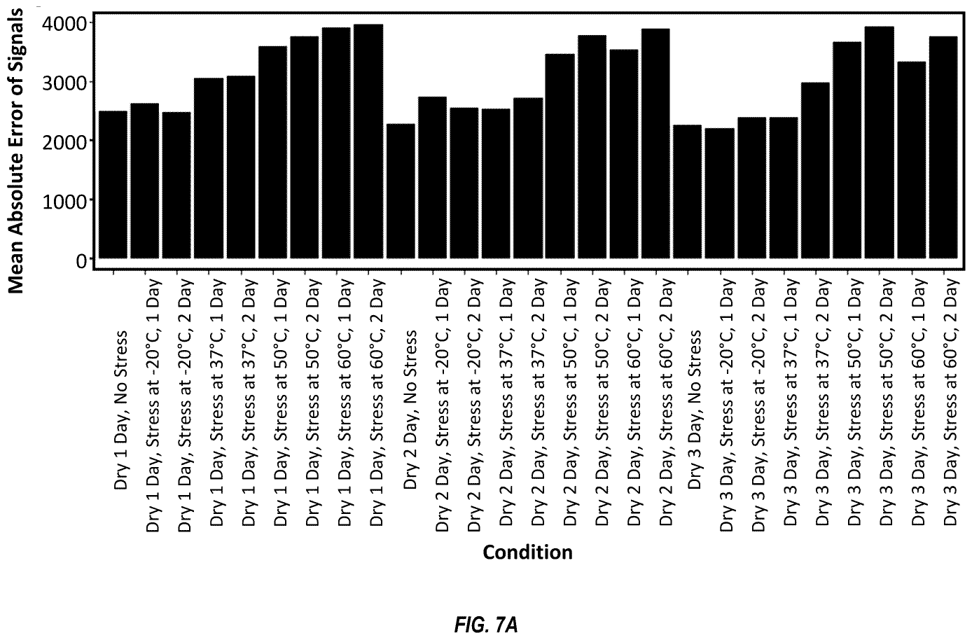

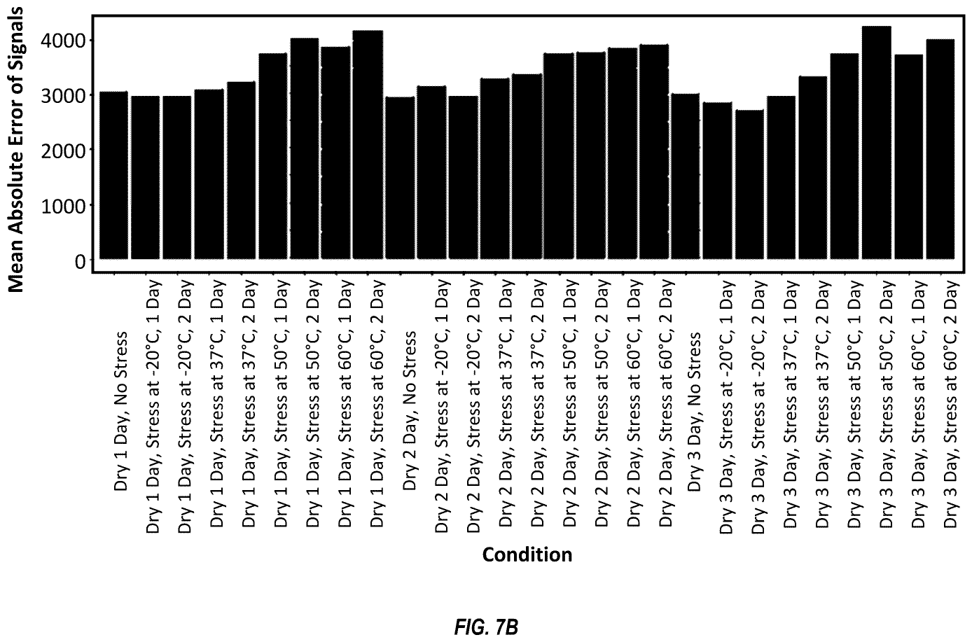

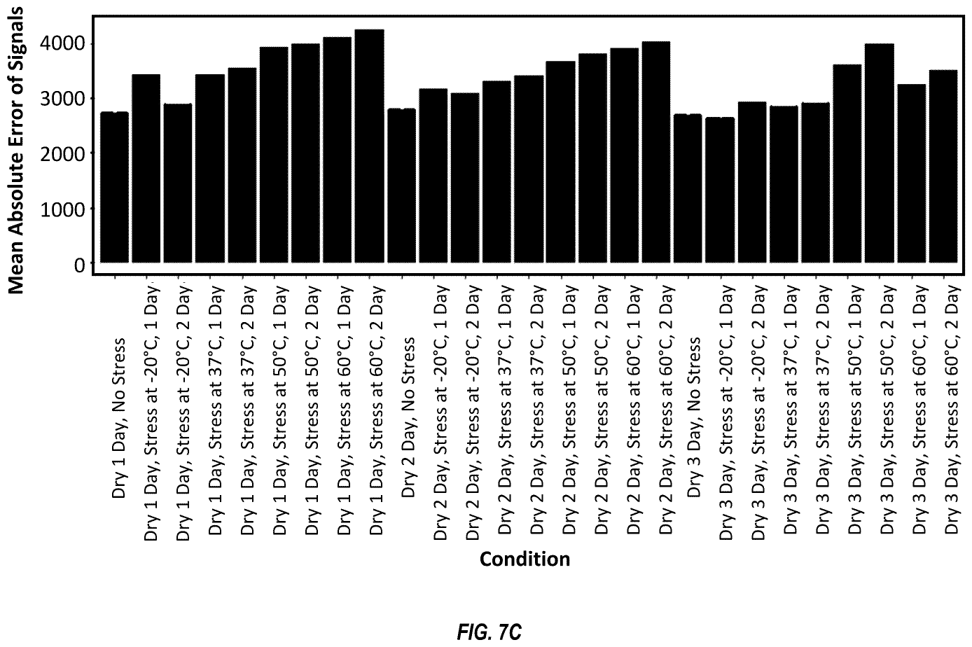

Plasma was dried at 4° C. and then exposed to −20° C., 37° C., 50° C., or 60° C. to temperature stress the samples and run in a multiplex assay compared to liquid plasma. FIG. 7A shows that drying at 4° C. for at least one day appears to protect samples from freezing. Drying plasma at 4° C. for at least two days appears to protect from freezing and at least one day's exposure to 37° C. FIG. 7B shows the results of plasma dried at room temperature followed by temperature stress. Drying at room temperature causes a decrease in concordance with liquid plasma when compared to drying at 4° C. The benefits of drying at −20° C. fall in between drying at 4° C. and room temperature as shown in FIG. 7C. Drying samples at 4° C., −20° C., and room temperature all provide suitable samples for multiplex assays.

### Example 9: Determination of Analytes from DBS Samples Measuring Changes in Plasma Proteins

### Study Methodology

A cohort of 16 healthy donors were sampled with three Tasso M20 smart sampling devices, one EDTA (plasma) BD vacutainer, and one serum BD vacutainer tube. After processing, plasma and serum samples were transferred to cryo-tubes and frozen at −80° C. Tasso M20 devices immediately had their drying tabs removed and were placed in a sealed plastic container with desiccant. One device from each donor was placed in the 4-8° C. refrigeration for 2 h before being packaged for shipping while the other two remained at room temperature. Packages were immediately shipped overnight to SomaLogic in Boulder. Two days post-draw, the refrigerated Tasso device as well as a device remaining at room temperature were opened and extracted in 80% serum diluent and 20% PBS for 1 h. Extracts were frozen at −80° C. at a concentration of 2.5%. The third Tasso device was held at room temperature for 7 days post-draw then extracted in a similar manner. The SOMAscan® assay was performed on all samples on the same plate to mitigate inter-assay variance. Serum and plasma samples were diluted according to a 20%, 0.5%, and 0.005% dilution protocol. Dried blood spots were diluted using a 2.5%, 0.05%, and 0.005% dilution protocol.

### Results

### Distribution of Correlations Between Matrices

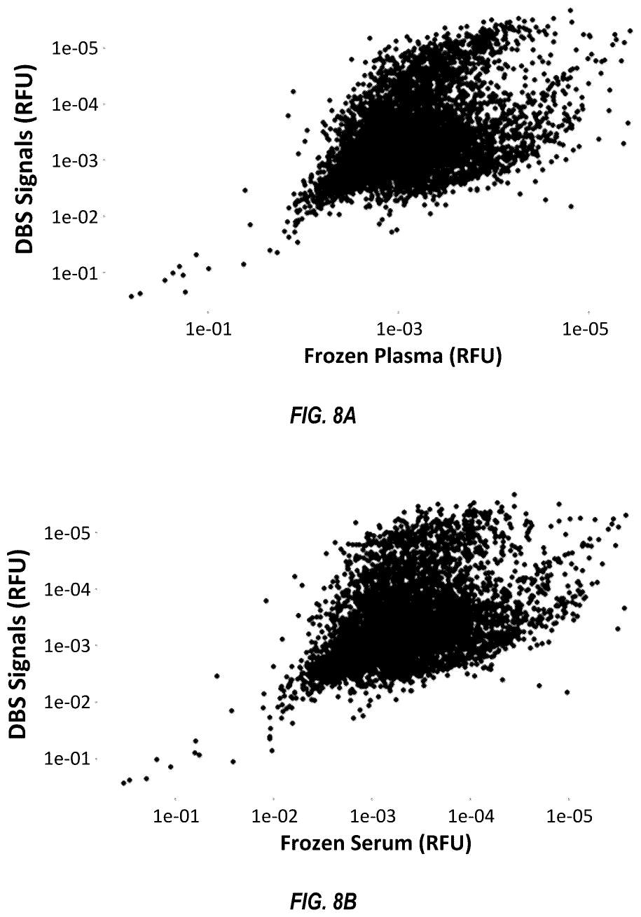

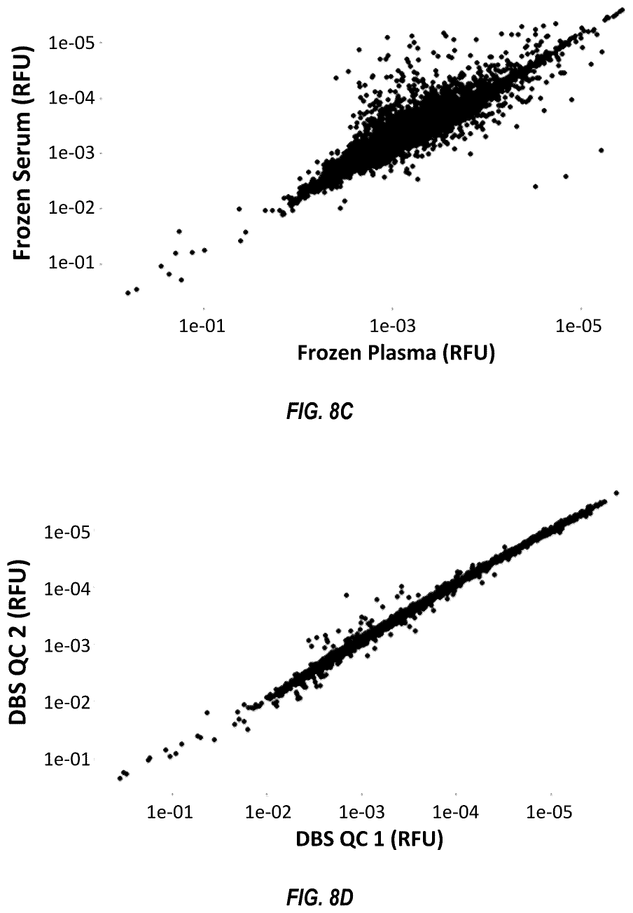

FIGS. 8A-8D compare the concordance between frozen plasma vs dried blood spots (8A), frozen serum vs dried blood spots (8B), frozen serum vs frozen plasma (8C), and two pooled DBS samples (8D). In the comparisons of (8A) and (8B), the cellular proteins in the DBS tend to overwhelm the signaling potential of proteins in plasma or serum that are often present in smaller quantities. This is in contrast to two DBS replicates made from pooled samples (8D) or even the matrices of frozen serum vs frozen plasma (8C).

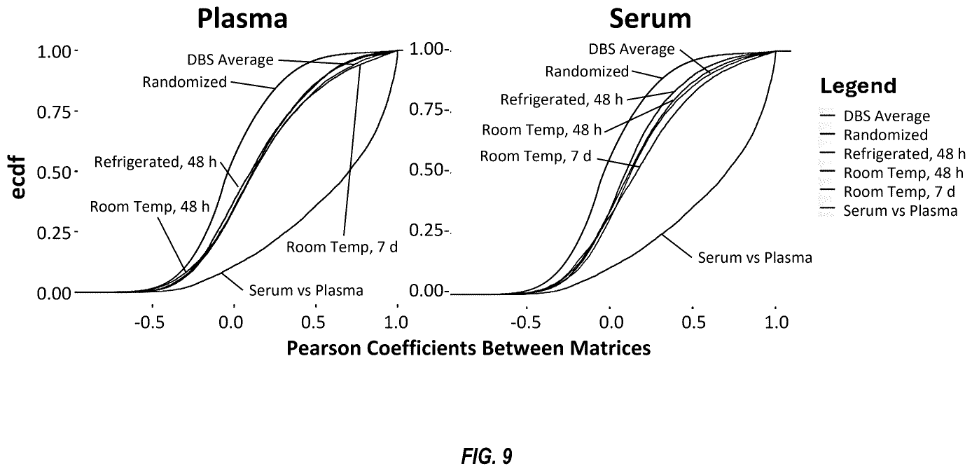

Based solely on data in FIGS. 8A-8D, it would be tempting to conclude that DBS is entirely discordant with both plasma and serum matrices. The addition of the cellular component of whole blood, which contains proteins which are in greater abundance than in plasma, causes the measurements of the majority of plasma proteins to be obscured in DBS. However, some proteins are so highly concentrated in plasma and/or depleted in cells that the abundance of additional protein does not obscure the signals from the liquid component of whole blood. FIG. 9 demonstrates that while most DBS signals are discordant with plasma, a fraction of them bear correlation between matrices. The cumulative distribution functions of all DBS Pearson correlation coefficients to frozen plasma are seen on the left along with those for serum on the right. For comparison purposes, correlations are included for plasma vs serum (direct and randomized). The masking effect of the cellular components of whole blood can be seen very clearly in FIG. 9 but a fraction of analytes appear to bear strong correlations to both plasma and serum.

### Number of Significant Correlations Between Serum Plasma and DBS

Statistically significant correlations between v4.1 analytes in plasma, serum and DBS were determined using two methods. In this example, analytes are defined as individual measurements in the SOMAscan® assay. In this way, some blood proteins are measured by multiple “analyte signals.”

One method (correlation significance through paired observations) involved using significance thresholds for p-values generated by a combination of similar observations. As FIG. 9 demonstrated, plasma and serum are not independent of each other, but correlated to one another. An observation in one matrix is informative to observations in the other. In addition, a similar study, referred to here as the “DBS Precision Study” (described below), looked at correlations between DBS made from anticoagulated, IV-drawn blood, and frozen plasma. The p-values from correlations found from each of these observations were combined and compared against a significance value of 0.1 (corrected for multiple comparisons and observations as described below). The results can be seen in Tables 1, 2, and 3.

The second method used to determine which of the correlations shown in FIG. 9 are significant employed the false discovery rate. Simulations involving the randomization of donors across matrices generated nearly three million spurious correlations for every DBS condition/matrix pairing. From these, the average number of false correlations for a menu of 7335 human analytes can be calculated for a given threshold of the Pearson correlation coefficient. The number of significant correlations is then calculated by the number of correlations with a coefficient above a threshold minus the average number of false correlations due to random chance. A detailed method can be found below and the results are given in Table 4.

### Number of Significant Correlations Using Paired Observations

The number of significant correlations between plasma/serum and DBS that occurs in various drying conditions is presented in the following tables using no paired observations, paired observations across plasma and serum, and paired observations across serum and plasma combined with results from the DBS Precision Study. Detailed methods can be found below in “Correlations through Paired Observations.” The “average” condition found in each of these tables is the result of treating all three conditions as technical replicates then taking the mean of the three correlations between DBS and plasma and serum.

TABLE 1
Number of Statistically Significant Correlations
Through Paired Observations

| | Room Temp | | Refrigerated | |
|---|---|---|---|---|
| | 48 h | 7 d | 48 h | Average |
| | No Combinations | | | |
| Serum | 129 | 233 | 89 | 150 |
| Plasma | 129 | 252 | 97 | 164 |
| | Combined Serum and Plasma Analysis | | | |
| Serum & Plasma | 295 | 486 | 217 | 385 |
| | Combined Serum and Plasma Analysis and | | | |
| | DBS Precision Study | | | |
| | 325 | 500 | 250 | 411 |

A significant increase in the number of significant correlations can be seen when paired observations are used as opposed to single correlations between DBS and serum or plasma. It should be noted that the Holm-Bonferroni correction used in this method is a conservative filter for false correlations when comparing across large numbers of analytes. By combining observations in serum and plasma, the number of significant correlations more than doubles. Adding the DBS Precision Study into the analysis increased the number of significant analytes by an average of 25 analytes. This small benefit is understandable considering the small cohort involved in that study.

TABLE 2
Statistically Significant Correlations Through Paired
Observations (Broken Down by Dilution Group)

| | | Room temp | | Refrigerated | |
|---|---|---|---|---|---|
| | | 48 h | 7 d | 48 h | Ave |
| | | No Combinations | | | |
| Serum | Dil 1 | 87 | 147 | 65 | 99 |
| | Dil 2 | 30 | 70 | 20 | 43 |
| | Dil 3 | 12 | 16 | 4 | 8 |
| Plasma | Dil 1 | 86 | 151 | 69 | 105 |
| | Dil 2 | 29 | 74 | 21 | 44 |
| | Dil 3 | 14 | 27 | 7 | 15 |
| | | Combined Serum and Plasma Analysis | | | |
| Serum & | Dil 1 | 158 | 274 | 141 | 207 |
| Plasma | Dil 2 | 91 | 158 | 55 | 121 |
| | Dil 3 | 47 | 55 | 22 | 58 |
| | | Combined Serum and Plasma Analysis and | | | |
| | | Precision Study | | | |
| | Dil 1 | 162 | 272 | 148 | 210 |
| | Dil 2 | 106 | 169 | 73 | 137 |
| | Dil 3 | 58 | 60 | 29 | 65 |

The number of significant correlations between serum and plasma and all three DBS conditions (top) were broken down into dilution group.

TABLE 3
Statistically Significant Correlations Through Paired
Observations (Broken Down by Dilution Group Coverage)

| | | Room temp | | Refrigerated | |
|---|---|---|---|---|---|
| | | 48 h | 7 d | 48 h | Ave |
| | | No Combinations | | | |
| Serum | Dil 1 | 1.4% | 2.4% | 1.1% | 1.6% |
| | Dil 2 | 2.7% | 6.3% | 1.8% | 3.9% |
| | Dil 3 | 6.3% | 8.4% | 2.1% | 4.2% |
| Plasma | Dil 1 | 1.4% | 2.5% | 1.1% | 1.7% |
| | Dil 2 | 2.6% | 6.7% | 1.9% | 4.0% |
| | Dil 3 | 7.4% | 14.2% | 3.7% | 7.9% |
| | | Combined Serum and Plasma Analysis | | | |
| Serum & | Dil 1 | 2.6% | 4.5% | 2.3% | 3.4% |
| Plasma | Dil 2 | 8.2% | 14.3% | 5.0% | 10.9% |
| | Dil 3 | 24.7.% | 28.9% | 11.6% | 30.5% |
| | | Combined Serum and Plasma Analysis and | | | |
| | | Precision Study | | | |
| | Dil 1 | 2.7% | 4.5% | 2.5% | 3.5% |
| | Dil 2 | 9.6% | 15.3% | 6.6% | 12.4% |
| | Dil 3 | 30.5% | 31.6% | 15.3% | 34.2% |

There are 6027 human analytes in dilution group 1, 1106 human analytes in dilution group 2, and 190 human analytes in dilution group 3. Proportionate to the total number of analytes in each group, the abundance of significant analytes increases as the dilution factor increases in the SOMAscan® assay. This could be due to the many excreted plasma proteins that are found in high abundance in dilution groups 2 and 3, which could be more easily read over interference from cellular analytes in whole blood.

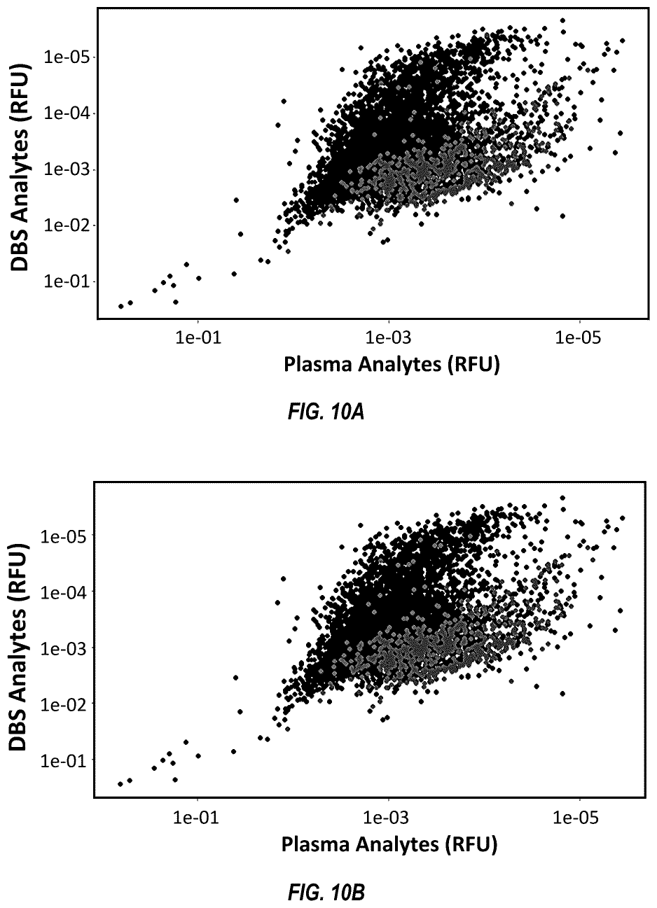

In FIG. 10A, signals from DBS that were held for 7 days prior to extraction plotted against signals from the corresponding signals frozen plasma. Analytes colored in gray were generated from paired observations in plasma, serum, and the DBS Precision Study (500 in total) were deemed to correlate to plasma. Plasma analytes cluster on the bottom half of the graph, which is consistent with previous analyses.

### Number of Significant Correlations Using an FDR Filter

The following results are determined by selecting an arbitrary false discovery rate (FDR) value and counting the number of analytes that meet the required Pearson coefficient threshold to achieve that value.

TABLE 4
Using the FDR to Filter the Number of Significant Correlations between DBS and Serum/Plasma

| | | | Serum | | | | Plasma | | | |
|---|---|---|---|---|---|---|---|---|---|---|
| | | FDR | 4.0% | 5.0% | 10.0% | 20.0% | 4.0% | 5.0% | 10.0% | 20.0% |
| Room Temp | 48 h | Sig Corr's | 315 | 365 | 538 | 754 | 266 | 327 | 533 | 795 |
| | | False Pos | 13 | 18 | 59 | 185 | 11 | 17 | 56 | 193 |
| | | Min Pearson | 0.72 | 0.69 | 0.6 | 0.5 | 0.75 | 0.71 | 0.61 | 0.5 |
| | 7 d | Sig Corr's | 393 | 501 | 755 | 1081 | 433 | 511 | 735 | 1002 |
| | | False Pos | 16 | 26 | 84 | 258 | 17 | 26 | 81 | 250 |
| | | Min Pearson | 0.77 | 0.72 | 0.61 | 0.49 | 0.76 | 0.72 | 0.62 | 0.5 |
| Refrigerated | 48 h | Sig Corr's | Limited | 154 | 264 | 386 | Limited | 146 | 348 | 621 |
| | | False Pos | Limited | 8 | 28 | 88 | Limited | 8 | 36 | 149 |
| | | Min Pearson | Limited | 0.78 | 0.68 | 0.59 | Limited | 0.8 | 0.68 | 0.56 |

| Average | Sig Corr's | 358 | 408 | 618 | 829 | 481 | 550 | 753 | 1034 |
|---|---|---|---|---|---|---|---|---|---|
| | False Pos | 14 | 20 | 68 | 198 | 19 | 27 | 81 | 254 |
| | Min Pearson | 0.74 | 0.71 | 0.61 | 0.51 | 0.71 | 0.68 | 0.59 | 0.48 |

In Table 4, all DBS conditions are compared against plasma and serum, and the number of correlations between matrices is given based on the tolerance for false positives within the population. The total population of analytes that will appear can be calculated by adding the significant correlations (Sig Corr's) to the false positives (False Pos). Dividing the number of false positives by this number gives the FDR. The minimum Pearson coefficient that a correlation can have and be considered significant is given by the “Min Pearson” value. Conditions and tolerances that yield greater than 500 analytes are marked in bold.

In FIG. 10B, signals from DBS that were held for 7 days prior to extraction plotted against signals from the corresponding frozen plasma. Analytes colored in gray were generated utilizing an FDR based algorithm (described below in Correlation Significance Filtered by False Discovery Rate) to generate 557 significant correlations. Plasma analytes cluster the same way for both methods (FIGS. 10A and 10B) indicating a high level of agreement between the two.

### Characterization of Analytes

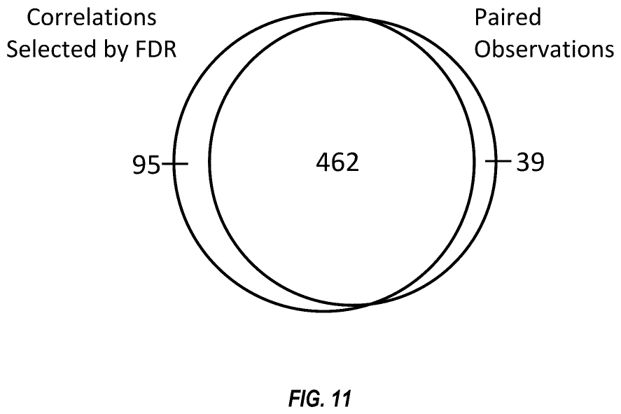

A comparison of the groups of analytes that are labeled as significant by each method shows considerable overlap (FIG. 11). This comparison provides confidence that one could use either method or combine methods to create a larger list of significant correlations.

In FIG. 11, the left area represents the significant correlations between frozen plasma and DBS (7 day drying time) selected by choosing a Pearson threshold that achieves an FDR of 5%. The right area represents the number of the same correlations using a three-way paired observation between the DBS and plasma and serum as well as observations from the DBS Precision Study. This overlap between the two results lends confidence that these analytes are in fact measuring plasma proteins.

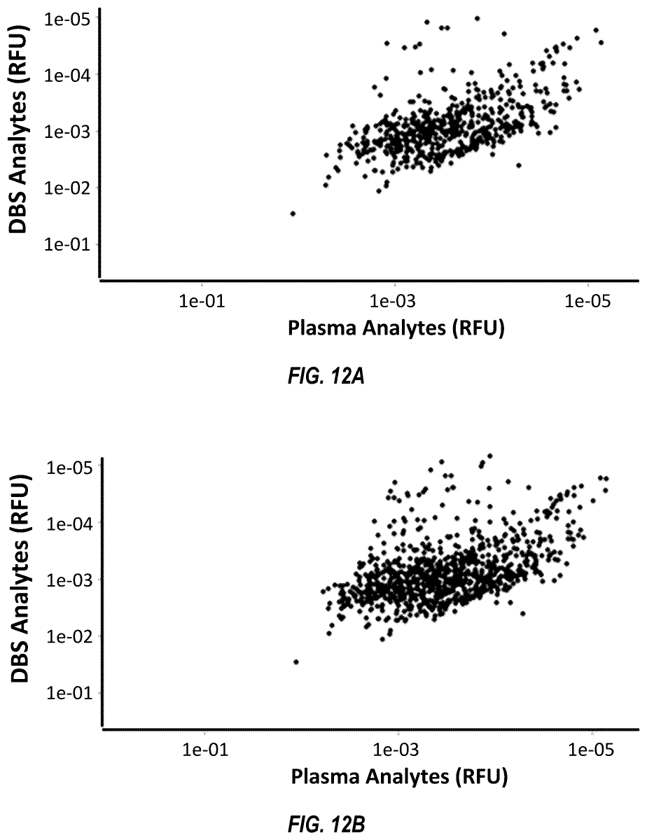

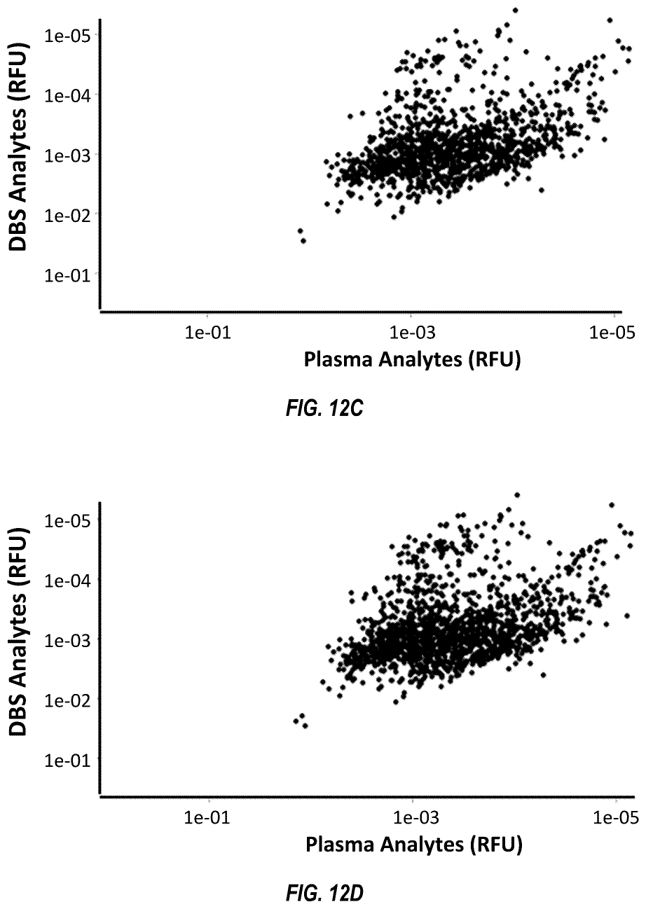

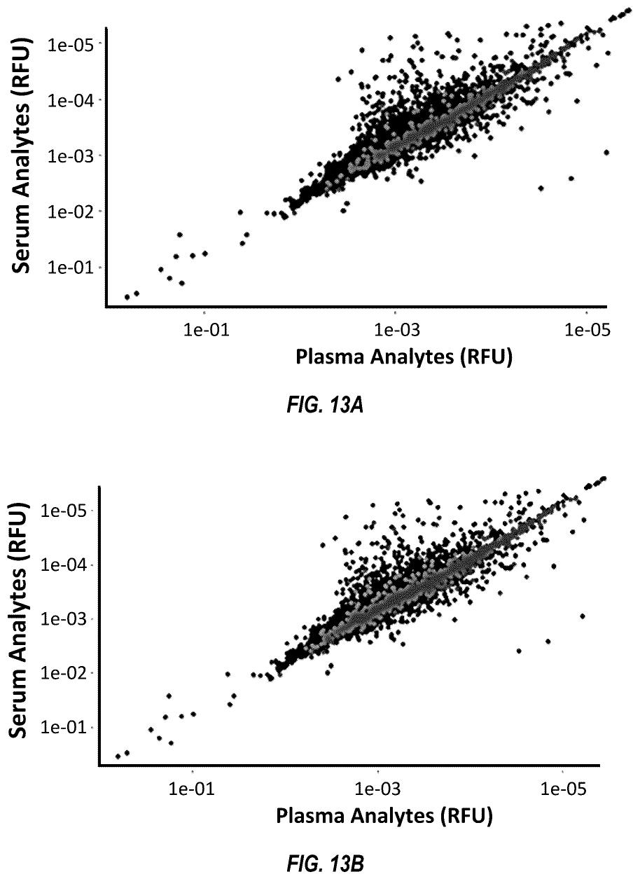

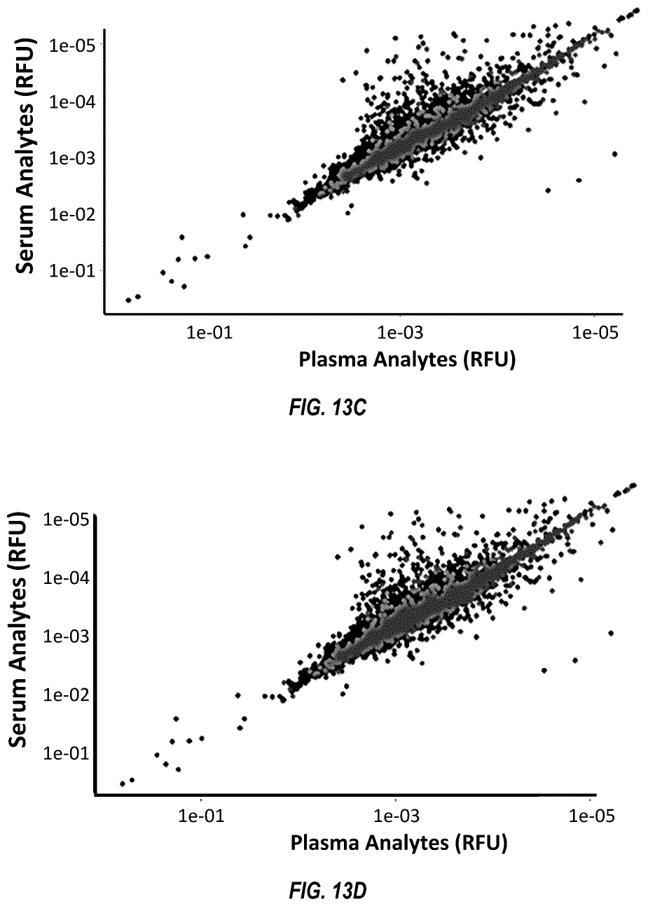

Once lists of significant correlations between DBS and plasma were generated, the character of plasma/serum analytes that are likely measurable in DBS were investigated. Concordance plots (FIGS. 12A-12D) between plasma and DBS that filter out all signals that are not significantly correlated, demonstrated a direct relationship between matrices for these select analytes. Analytes with lower signal strength appeared to have a less-linear relationship to DBS. Without wishing to be bound to a particular theory, this could be due background from DBS making a more significant impact on plasma signals of less magnitude. When significant analytes were mapped against concordance plots between serum and plasma (FIGS. 13A-13D), it became clear that analytes having a greater concordance between those two matrices also have a greater chance of correlating between plasma/serum and DBS.

As shown in FIGS. 12A-12D, each of the concordance plots compares the relationship between plasma and DBS signals in analytes that have been characterized as having a significant correlation to between DBS (7 day drying) and frozen plasma. As the allowable FDR increases: (12A) 5%, (12B) 10%, 12C) 15% and (12D) 20%; the number of analytes increase as well.

As shown in FIGS. 13A-13D, analytes in gray have Pearson coefficients (DBS dried for 7 days vs frozen plasma) that are greater than a threshold that achieve an FDR of (13A) 5%, (13B) 10%, (13C) 15% and (13D) 20%. These are mapped against a concordance plot of serum vs plasma signals. FIGS. 13A-13D show that the more concordant analytes are between plasma and serum, the more likely they are to be concordant between plasma and DBS.

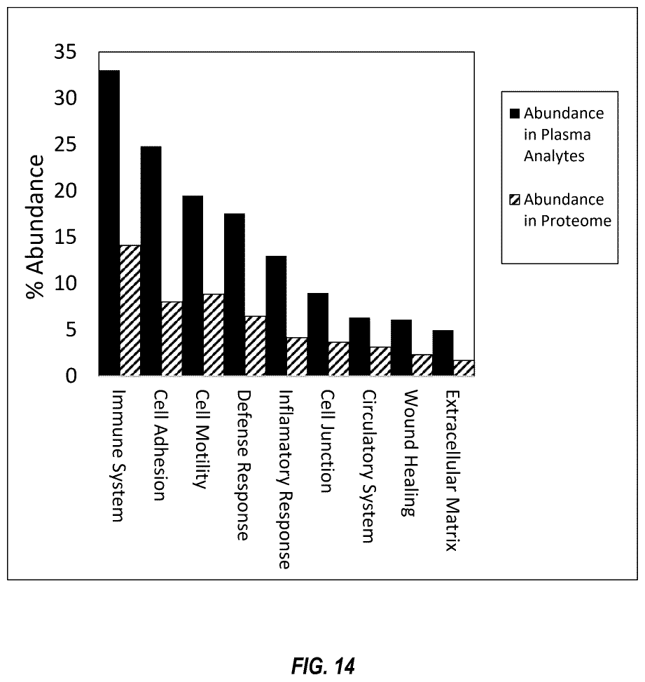

Another way to classify the list of significant correlations is by classifying the functions of the analytes that measure plasma proteins. Princeton University compiled a list of gene-ontology terms for the human proteome. One way to use the Princeton University dataset is to compare the abundance of the selected plasma analytes associated with each category with the abundance of the analytes in the human proteome. FIG. 14 shows that proteins used in the plasma measurements are involved in immunology, cell adhesion, cell motility, defense response, inflammatory response, cell junctions, the circulatory system, wound healing, and the extracellular matrix. The percentage of plasma analytes (left) that fall into various categories of biological function is compared with the abundance of proteins that have been sorted into those categories in the entire human proteome (right). Each of these categories is selected for having an enriched representation (greater than 2×) in the plasma proteins identified by paired observations and FDR filtering.

### Methods

### Correlations Through Paired Observations:

There are multiple observations that can be harnessed to address the question of how many plasma/serum analytes bear significant correlations to whole blood. Each patient in this study was sampled via two additional matrices other than DBS (frozen plasma and serum) to compare with each their DBS samples. Plasma and serum are not independent matrices, over 2500 analytes demonstrated a significant correlation in this cohort between the two matrices (α=0.1, Bonferroni-holm corrected). Therefore, the probability that a correlation between plasma and DBS is significant can be modified with the measured significance (p-value) of that same correlation between serum and DBS.

A separate study (referred to as the DBS Precision Study), involving a small cohort of six donors were drawn by IV into EDTA vacutainer tubes and their blood was divided into five assay replicates. Plasma was also collected from these donors and three replicates were analyzed to capture which plasma analytes could be measured in whole blood. Plasma analytes that correlate to DBS in the DBS Precision Study can support similar observations in this study. A detailed description of the methods for this study are provided below in the protocol section. Analyte signals were median normalized.

To discount the null hypothesis that an individual correlation is due to random chance, a method was developed that rewards consistent observations across matrices and experiments. A positive correlation was determined using a Pearson coefficient cut-off that was determined by calculating correlations between matrices after randomization (so that donors are no longer matched) across the entire library. This was done 401 times for a total of 2.94 million random correlations. This number was considered sufficient as repeated computations indicated a high degree of precision.

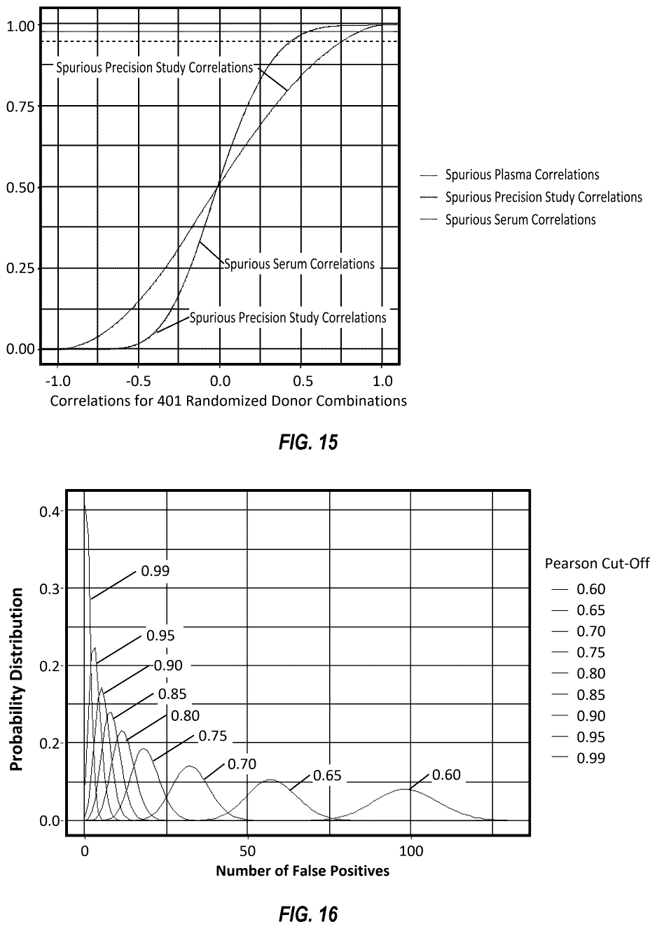

FIG. 15 shows a Cumulative Distribution Function (CDF) plot of distribution of Pearson coefficients generated by comparing random plasma/serum values with DBS dried for seven days. The dotted line represents the cut off where 95% of random Pearson coefficients fall (0.45). The solid line represents the cut off where 98% of random Pearson coefficients fall (0.56). The chances that randomizing the DBS and plasma of 6 individuals for the DBS Precision Study will result in a casual alignment of donors is much higher than with 16 individuals so the 95% and 98% thresholds are represented by much larger Pearson coefficients (0.75 and 0.86 respectively). Given these restrictive values, 95% threshold was used.

A Pearson coefficient must be greater than a 98% cut-off for plasma and serum correlations or a 95% cut-off (0.75) for DBS Precision Study correlations to be considered as a correlation. There was functionally no difference in the results obtained from a 98% or 95% cut-off in the 16 donor study so the more stringent threshold was used. The values of the Pearson coefficient thresholds were determined individually between all matrices and conditions. Correlations with a correlation coefficient below the threshold were manually assigned a p-value of 1. The resulting p-values between paired plasma and serum correlations are multiplied together to obtain the probability of both results being due to random chance. This is illustrated by Table 5.

TABLE 5
Example Paired Observation Algorithm

| Seq ID | Serum vs Room Temp DBS, p-value | Serum vs Room Temp DBS, p-value | Plasma vs Room Temp DBS, Pearson | Plasma vs Room Temp DBS, p-value | Combined p-value | $< \frac{{α}^{2}}{\sim 7335}$ |
|---|---|---|---|---|---|---|
| seq. 10382.1 | 0.495 | 1 | 0.555 | 0.026 | 0.026 | No |
| seq. 10390.21 | −0.264 | 1 | −0.269 | 1 | 1 | No |
| seq. 10391.1 | 0.740 | 0.001 | 0.846 | 3.59E−05 | 3.79E−08 | Yes* |
| seq. 10398.110 | −0.041 | 1 | −0.027 | 1 | 1 | No |
| seq. 10418.36 | 0.544 | 0.029 | 0.254 | 1 | 0.029 | No |
| seq. 10419.1 | 0.493 | 1 | 0.436 | 1 | 1 | No |
| seq. 10420.30 | −0.066 | 0 | −0.043 | 1 | 1 | No |
| seq. 10424.31 | 0.911 | 9.53E−07 | 0.914 | 7.34E−07 | 7.00E−13 | Yes# |
| seq. 10425.3 | −0.154 | 1 | 0.071 | 1 | 1 | No |
| seq. 10426.21 | −0.120 | 1 | −0.021 | 1 | 1 | No |
| seq. 10428.1 | 0.548 | 0.028 | 0.557 | 0.025 | 0.000698 | No# |

Threshold > 0.52
Threshold > 0.53

A two-comparison version of the algorithm is shown in Table 5 as an example. In this representation of paired observation algorithm, the thresholds for a significant correlation are shown at the bottom of the table. Pearson coefficients beneath the thresholds are highlighted in italics, while those above the cut-off are highlighted in bold. The p-values of Pearson coefficients that lie below the acceptance threshold are given a value of 1 so that when they are multiplied with their partnering value in the other matrix, they do not contribute to the combined probability of spurious correlations. The resulting p-values are compared to a Bonferroni-Holm corrected α² (α=0.1). In this algorithm α is raised to the power of the number of correlations being combined (in this case 2). In this way, a highly significant correlation in only one matrix is sufficient to classify a plasma analyte. Alternatively, multiple weaker correlations can combine to identify plasma analytes. Decreasing the alpha by the power of the number of correlations prevents p-values from simply combining together until random chance makes them significant. The result indicated by * is an example of an analyte that would not have cleared the bar for acceptance using only plasma or serum correlations. The values indicated by # would have remained unchanged.

### Correlation Significance Filtered by False Discovery Rate:

The null hypothesis can also be rejected by requiring that the set of all significant correlations within a dataset contain a maximum false discovery rate of spurious correlations. Using randomized correlations as described above, a Pearson coefficient threshold can be selected that tunes the false discovery rate of spurious correlations. Taking a specific example of random correlations between plasma and 7-day dried DBS one can see that 99.9% of Pearson coefficients are less than 0.87. If that cut-off is used in this example one would expect the false positive rate, or chance that an individual correlation would be due to chance would be 1 in 1000. If analyte correlations are sorted from greatest to smallest Pearson coefficient, the average number of false correlations in a group with a minimum coefficient value can be determined. Dividing by the number of analytes in this group gives a false discovery rate (FDR).

The following method determines how many significant analytes can be binned as having significant correlations between matrices at varying levels of confidence. The distribution of probabilities for false positives or spurious correlations is given by equation 1.

$$\begin{matrix}Binomial Probability Distribution for False Correlations & \\ ( \begin{matrix}n \\ k\end{matrix} ) {{p}^{k} ( 1 - p )}^{n - k} & Equation 1\end{matrix}$$

$$( \begin{matrix}7335 \\ FP\end{matrix} ) {{FPR}^{FP} ( 1 - FPR )}^{7335 - FP}$$

n=The number of analytes being compared (7335 human analytes)
k=The number of false positives (FP) or spurious correlations.
p=The false positive rate (FPR) for correlations between matrices.
   The false positive rate is determined by choosing a threshold for the 2.9 million random Pearson coefficients generated by random decoupling of donor matrices and finding the percentage of random coefficients that occur above the cut-off.

Probability curves showing different probability density functions for the possible number of falsely correlated analytes when comparing plasma to DBS samples that have been dried for 7 days were generated using Pearson cut-offs of 0.60, 0.65, 0.70, 0.75, 0.80, 0.85, 0.90, 0.95, and 0.99 are shown in FIG. 16. The higher the cut-off for the allowable Pearson coefficients, the lower the number of false positives. These curves can be used to determine the weighted average for the number of false positives that would be expected given a specific threshold.

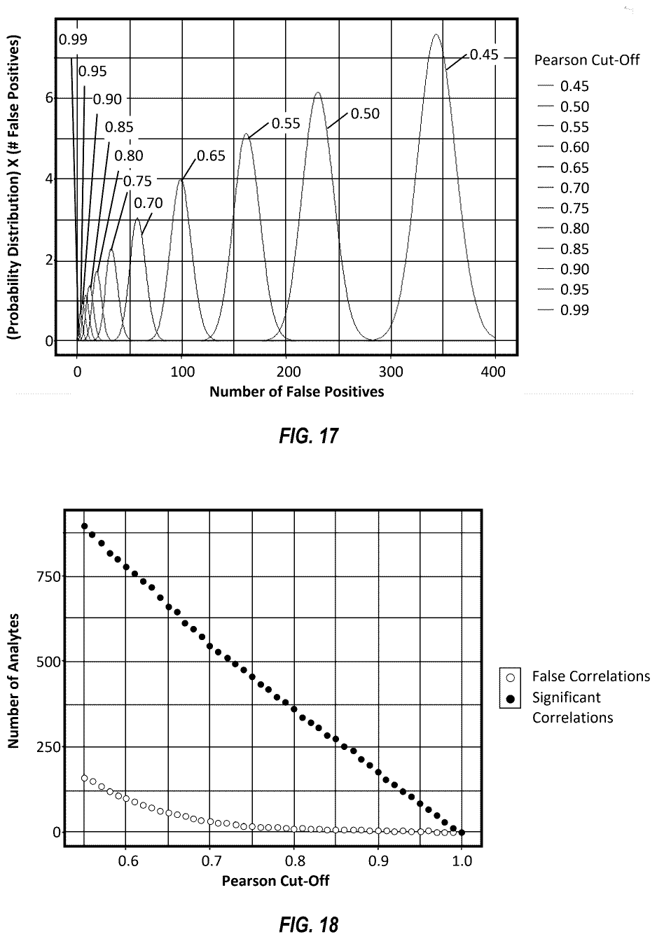

The weighted average of false correlations was found by multiplying the number of false positives by their probability density functions (as shown in FIG. 17) and then finding the resulting area of the curve (equation 2).

$$\begin{matrix}Weighted Average of False Correlations & \\ FP Ave = {\int }_{0}^{∞} FP * ( \begin{matrix}7335 \\ FP\end{matrix} ) {{FPR}^{FP} ( 1 - FPR )}^{7335 - FP} dFP & Equation 2\end{matrix}$$

FPR=the false positive rate or correlations between matrices
FP=the number of false positives for a given FDR

This integral can only be estimated with whole number values of FP, using the trapezoidal rule for integration to find the area under the curve. In practical terms an accurate measure of the integral can be determined by bounding the integral to a region where the curves are significantly above the x-axis.

The weighted average of false correlations for a given threshold of Pearson coefficients is given in open circles and is derived from the area under the curve of the number of false correlations multiplied the probability density function (equation 2 and FIG. 18). The number of significant correlations (closed circles) is sum total of all correlations between a matrix and DBS samples (in this case 7 day drying DBS) with a Person coefficient above a cut-off and subtracting the number of false correlations. The number of significant correlations appears to increase linearly.

$$\begin{matrix}False Discovery Rate for Spurious Correlations & \\ FDR = \frac{Average False Positives}{Total Correlations} & Equation 3\end{matrix}$$

Total Correlations=The number of correlations between a matrix and DBS condition that have Pearson coefficient above a given threshold
Average False Positives=The weighted average of false positives given by equation 2.

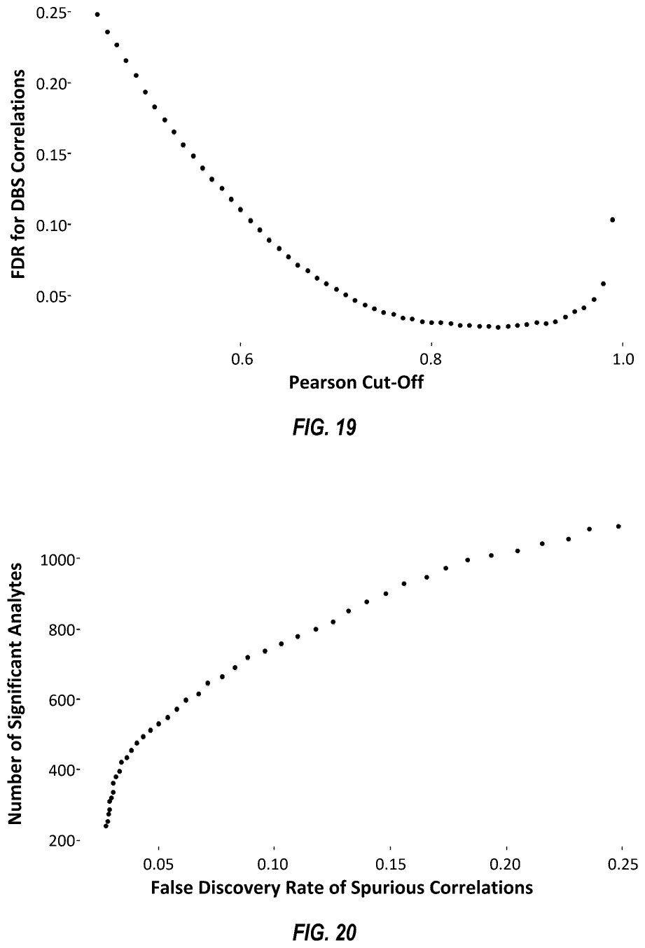

The false discovery rate for spurious correlations (calculated by equation 3) is shown in FIG. 19 and clearly demonstrates the dangers of arbitrarily high cut-offs for Pearson coefficients when trying to eliminate false correlations. Any Pearson threshold that moves the FDR beyond its minimum both decreases the number of analytes and increases the ratio of false correlations.

Every FDR (x-axis) can be mapped to the number of significant analytes (total correlations−false correlations) as shown in FIG. 20. The curve shown in FIG. 20 was made from correlations between plasma and DBS that was allowed to dry for 7 days. Values for Table 4 were calculated in this way.

### Protocol for DBS Precision Study

Whatman 903™, Guthrie cards were used to dry and extract whole blood samples to align closely with a device that might be used in the field. Whole blood spots were allowed to dry before being cut out of their card and extracted in plasma diluent and PBS. A concentration of 2.5% extract was used, allowing a volume of over 1 mL of extraction buffer. This minimized the percentage of volumetric distortions created by different sized Guthrie card cut-outs. After 1 h the extracts were transferred to matrix tubes and frozen at −80° C. On the day of the assay, samples were thawed and diluted to concentrations of 2.5%, 0.05%, and 0.005%. By the end of the extraction, Guthrie cut-outs appeared completely clean of whole blood. Plasma samples were immediately frozen at −80° C. and diluted according to standard procedure.

1. A method of preparing a biological sample for a multiplex assay comprising:
depositing the biological sample comprising a plurality of target molecules onto a collection device;
drying the biological sample on the collection device for a period of time to stabilize the dried sample prior to any temperature fluctuation;
wherein the biological sample is dried for at least 4 hours at about room temperature or below room temperature so that target molecules in the dried biological sample are detectable in the multiplex assay.

2. The method of claim 1, wherein the biological sample is dried at about room temperature, 4° C.-8° C., or −20° C.

3. The method of claim 1, wherein the dried biological sample is stored at about room temperature or below room temperature prior to detection in the multiplex assay.

4. The method of claim 1, wherein the dried biological sample is stored at about 4° C.-8° C., or −20° C. and/or the biological sample is dried for at least 6 hours, at least 8 hours, at least 10 hours, at least 12 hours, at least 14 hours, at least 16 hours, at least 18 hours, at least 20 hours, at least 22 hours, one day, at least two days, or at least three days.

5. (canceled)

6. The method of claim 1, wherein the biological sample is selected from plasma, serum, urine, whole blood, leukocytes, peripheral blood mononuclear cells, buffy coat, sputum, tears, mucus, nasal washes, nasal aspirate, semen, saliva, peritoneal washings, ascites, cystic fluid, meningeal fluid, amniotic fluid, glandular fluid, lymph fluid, nipple aspirate, bronchial aspirate, bronchial brushing, synovial fluid, joint aspirate, organ secretions, cells, a cellular extract, and cerebrospinal fluid.

7. The method of claim 1, wherein the biological sample is selected from plasma, serum, urine, and whole blood and/or wherein the plurality of target molecules are selected from a protein, a peptide, a carbohydrate, a polysaccharide, a glycoprotein, a hormone, a receptor, an antigen, an antibody, a virus, a bacteria, a metabolite, a cofactor, an inhibitor, a drug, a dye, a nutrient, a growth factor, a cell and a tissue.

8. (canceled)

9. The method of claim 1, wherein the dried biological sample is homogenized by the drying process.

10. The method of claim 1, wherein the plurality of target molecules are extractable from the collection device after the biological sample is dried and detectable in the multiplex assay.

11. A method of detecting a plurality of target molecules comprising:
extracting target molecules in a dried biological sample from a collection device;
diluting the extracted target molecules into a first dilution and a second dilution;
contacting the first dilution with a first capture reagent to form a first capture reagent affinity complex with its target molecule if the target molecule is present in the first dilution;
contacting the second dilution with a second capture reagent to form a second capture reagent affinity complex with its target molecule if the target molecule is present in the second dilution;
incubating the first and second dilution samples separately to allow capture reagent affinity complex formation; wherein each of the first capture reagent affinity complex and the second capture reagent affinity complex are immobilized on separate first solid supports;
releasing and capturing the first capture reagent affinity complex on a second solid support;
after releasing the first capture reagent affinity complex, releasing and capturing the second capture reagent affinity complex on the second solid support; and
detecting for the presence of or determining the level of the first capture reagent, second capture reagent of the first or second capture reagent affinity complexes, or the presence or amount of the first or second capture reagent affinity complexes.

12. The method of claim 11, wherein the target molecules are extracted from the collection device in a formulation for at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 70 minutes, at least 80 minutes, at least 90 minutes, at least 100 minutes, at least 110 minutes, at least 120 minutes, at least 130 minutes, at least 140 minutes, at least 150 minutes, at least 160 minutes, at least 170 minutes, at least 180 minutes, at least 190 minutes, or at least 200 minutes.

13. (canceled)

14. The method of claim 12, wherein the formulation comprises a buffering agent, one or more salts, a chelating agent, a protease inhibitor, a non-ionic surfactant, and an oligonucleotide.

15. The method of claim 14, wherein the one or more salts are each independently selected from a sodium salt, a potassium salt and a magnesium salt.

16. (canceled)

17. The method of claim 15, wherein the sodium salt is NaCl, the potassium salt is KCl and the magnesium salt is MgCl₂.

18-34. (canceled)

35. The method of claim 11, wherein the target molecule is an oligonucleotide, and optionally wherein the oligonucleotide is a single stranded oligonucleotide.

36. The method of claim 35, wherein the oligonucleotide is from 20 to 100 nucleotides in length, or from 25 to 80 nucleotides in length, or from 25 to 70 nucleotides in length or from 25 to 50 nucleotides in length or about 30 nucleotides in length.

37. The method of claim 35, wherein the oligonucleotide comprises one or more modified nucleotides.

38-63. (canceled)

64. The method of claim 11, further comprising contacting a third dilution sample with a third capture reagent, wherein a third capture reagent affinity complex is formed by the interaction of the third capture reagent with its target molecule if the target molecule is present in the third dilution sample; wherein the third dilution sample is incubated separately from the first and second dilution samples to allow capture reagent affinity complex formation of the third aptamer with its target molecule.

65-81. (canceled)

82. A method for preparing a liquid sample comprising:
drying the sample at a constant temperature of from −20° C. to room temperature for at least four (4) hours to create a dried sample; and
reconstituting the dried sample with a formulation comprising a buffering agent, one or more salts, a chelating agent, a protease inhibitor, a non-ionic surfactant, and an oligonucleotide.

83. The method of claim 82, wherein the one or more salts are each independently selected from a sodium salt, a potassium salt and a magnesium salt.

84-110. (canceled)

111. The method of claim 82, wherein the sample is diluted to a first dilution and a second dilution, wherein the first dilution is a dilution of the test sample of from 0.001% to 0.009% (or is 0.001%, 0.002%, 0.003%, 0.004%, 0.005%, 0.006%, 0.007%, 0.008% or 0.009%) or is from 0.002% to 0.008% or is from 0.003% to 0.007% or is about 0.005%, and the second dilution is a dilution of the test sample of from 0.01% to 1% (or is 0.01%, 0.02%, 0.03%, 0.04%, 0.05%, 0.06%, 0.07%, 0.08%, 0.09%, 0.1%, 0.15%, 0.2%0, 0.25%, 0.3%, 0.35%, 0.4%, 0.45%, 0.5%, 0.6%, 0.7%, 0.8%, 0.9% or 1%) or is from 0.1% to 0.8% or is from 0.2% to 0.75% or is about 0.5%.

112-124. (canceled)

125. A composition comprising:
dried sample and a formulation comprising a buffering agent, one or more salts, a chelating agent, a protease inhibitor, a non-ionic surfactant, and an oligonucleotide;
wherein, the dried sample was derived from a liquid sample dried at a constant temperature of from about −20° C. to room temperature for at least four (4) hours.

126-130. (canceled)
